(19) United States
(12) Patent Application Publication
(10) Pub. No.: US 2018/0105825 A1
(43) Pub. Date: Apr. 19, 2018

(54) SYNTHETIC REPRESSION OF GENE EXPRESSION IN PLANTS

(71) Applicant: Colorado State University Research Foundation, Fort Collins, CO (US)

(72) Inventors: June I. Medford, Wellington, CO (US); Mauricio S. Antunes, Fort Collins, CO (US); Tessema K. Kassaw, Fort Collins, CO (US)

(73) Assignee: Colorado State University Research Foundation, Fort Collins, CO (US)

(21) Appl. No.: 15/787,562

(22) Filed: Oct. 18, 2017

Related U.S. Application Data
(60) Provisional application No. 62/409,555, filed on Oct. 18, 2016.

(51) Int. Cl. C12N 15/82

(52) U.S. Cl. CPC C12N 15/8218; C12N 15/8227; C12N 15/8225; C12N 15/8223

(57) ABSTRACT

The present disclosure provides synthetic repressor constructs and the proteins encoded therein, as well as synthetic repressible promoter constructs for use in combination with the synthetic repressor constructs/synthetic repressors disclosed herein. Various combinations of synthetic repressor constructs and synthetic repressible promoter constructs are also provided in synthetic genetic circuits for modifying expression of a protein of interest in a plant cell.

### CROSS REFERENCE TO RELATED APPLICATIONS

This application claims priority to U.S. provisional application No. 62/409,555, filed Oct. 18, 2016, the disclosure of which is hereby incorporated by reference in its entirety.

### GOVERNMENTAL RIGHTS

This invention was made with government support under grant DE-AR0000311 awarded by the Department of Energy and grant W911NF-09-10526 awarded by the US Department of Defense, Defense Threat Reduction Agency. The government has certain rights in the invention.

### FIELD OF THE INVENTION

The present disclosure provides synthetic repressor constructs and the proteins encoded therein, as well as synthetic repressible promoter constructs for use in combination with the synthetic repressor constructs/synthetic repressors disclosed herein. Various combinations of synthetic repressor constructs and synthetic repressible promoter constructs are also provided in synthetic genetic circuits for modifying expression of a protein of interest in a plant cell.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

As plant biotechnologists desire to install more transgenes together, the transcriptional control of these genes becomes increasingly important. Numerous synthetic genetic systems allowing controlled activation, and thereby increased levels, of gene expression exist. However, some applications require that a gene be expressed at high levels than repressed. Accordingly, there remains a need in the art for novel, synthetic genetic circuits in plants that function independently from or even replace endogenous networks.

### SUMMARY OF THE INVENTION

In an aspect, the present disclosure encompasses a synthetic repressor construct for modifying gene expression in a plant, comprising a nucleic acid encoding a transcriptional repressor domain linked to a DNA-binding domain, wherein the transcriptional repressor domain and the DNA-binding domain are operable in the same plant species.

In another aspect, the present disclosure encompasses a synthetic repressible promoter construct for use in combination with a synthetic repressor construct, the synthetic repressible promoter construct comprising: (a) a nucleic acid sequence encoding a core promoter capable of conferring constitutive gene expression in a plant species, the core promoter optionally comprising a TATA box; and (b) a synthetic regulatory element comprising at least one copy of a binding element having a nucleic acid sequence capable of specifically binding its cognate DNA-binding domain, the copy of the at least one binding element inserted at a position upstream of the core promoter, downstream of the core promoter but before the translation start site for a protein of interest, or proximal to the 5′ end of the optionally present TATA box when present.

In another aspect, the present disclosure encompasses an artificial genetic circuit for modifying expression of a protein of interest in a plant, comprising: a promoter operably linked to a synthetic repressor construct, the synthetic repressor construct comprising a nucleic acid encoding a transcriptional repressor domain linked to a DNA-binding domain; a nucleic acid construct comprising a nucleic acid encoding a protein of interest; a synthetic repressible promoter construct operably linked to the nucleic acid encoding the protein of interest, the synthetic repressible promoter construct comprising: (i) a nucleic acid sequence encoding a core promoter capable of conferring constitutive gene expression in a plant species, the core promoter optionally comprising a TATA box, and (ii) a synthetic regulatory element comprising at least one copy of a binding element having a nucleic acid sequence capable of specifically binding the DNA-binding domain of the synthetic repressor, the copy of the at least one binding element inserted at a position upstream of the core promoter, downstream of the core promoter region but before a translation start site for the protein of interest, or proximal to the 5′ end of the optionally present TATA box when present; and wherein the transcriptional repressor domain of the synthetic repressor, the DNA-binding domain of the synthetic repressor, the promoter operably linked to the synthetic repressor construct, and the core promoter of the synthetic repressible construct are each operable in the same plant species.

In another aspect, the present disclosure encompasses a transgenic plant cell comprising any one of a synthetic repressor construct disclosed herein, a synthetic repressible promoter construct disclosed herein, or a synthetic genetic circuit disclosed herein, or any combination thereof.

In another aspect, the present disclosure encompasses a transgenic plant comprising any one of a synthetic repressor construct disclosed herein, a synthetic repressible promoter construct disclosed herein, or a synthetic genetic circuit disclosed herein, or any combination thereof.

In another aspect, the present disclosure encompasses a kit comprising any one of a synthetic repressor construct disclosed herein, a synthetic repressible promoter construct disclosed herein, or a synthetic genetic circuit disclosed herein, or any combination thereof.

In another aspect, the present disclosure encompasses a method for modifying expression of a protein of interest in a plant, the method comprising introducing a synthetic genetic circuit as disclosed herein, into a cell of the plant, wherein the promoter operably linked to the synthetic repressor construct is an inducible promoter.

In another aspect, the present disclosure encompasses a method for creating a library comprising a plurality of synthetic repressible promoter constructs, the method comprising: providing a construct comprising a core promoter capable of conferring constitutive gene expression in a plant species, wherein the promoter optionally comprises a TATA box, and modifying the construct a plurality of times by (a) introducing one or more copies of a binding element having a nucleic acid sequence capable of specifically binding a DNA-binding domain, the copy of the binding element inserted at a position upstream of the core promoter, downstream of the core promoter but before a translation start site for a protein of interest, or proximal to the 5′ end of the optionally present TATA box when present, and then (b) varying the number of binding elements at a given position and/or the spacing between the 2 or more binding elements at a given position.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A-B depict schematic diagrams of various constructs used in the protoplast experiments detailed in the Examples. (A) Synthetic repressible promoter architecture, containing repressor binding sites (operators) placed upstream (cyan), downstream (orange), or just upstream of the TATA-box (green) of base promoters. The number (2×, 4×, 5×, 8×), spacing between binding sites (SP10, 10-nt spacer represented by horizontal black bar connecting binding sites), and type of binding sites (Gal4 or LexA) are indicated. (B) Genetic circuit used for testing promoter-repressor combinations in protoplasts. An external inducer (DEX or OHT) activates transcription of a repressor protein as well as Firefly Luciferase (F-luc) through the same promoter. F-luc luminescence thus represents the concentration of the repressor inside the cell. The repressor protein represses a constitutively active repressible promoter that controls transcription of Renilla Luciferase (R-luc). The strength of repression is measured by the decrease in R-luc luminescence as F-luc luminescence increases on the addition of more inducer.

FIG. 2A-F depicts graphs of the noise in the protoplast data. (A, B) Greyscale heat map representing measured F-luc luminescence intensities with no inducer present (i.e., basal level expression) for different biological (vertical axis) and technical (horizontal axis) replicates. (A) OHT- and (B) DEX-based promoter-repressor pairs are plotted separately. (C, D) Standard curve for luminescence produced (RLU/(area*s) as a function of total number of molecules for (C) F-luc and (D) R-luc. Lines represent best fits. (E) Scatter plot of R-luc and F-luc luminescence of a repressible promoter without a repressor (beta plasmid), measured on different days. Open circles represent DEX-based inducible promoter, closed circles represent OHT-based inducible promoter. The same repressible promoter was used in both circuits. (F) Magnitude of the standard deviation corresponding to the three different sources of noise: within a 96-well plate, between different transformations, and between different preparations of protoplasts (i.e. batches).

FIG. 3A-F depicts graphs illustrating the variation due to different protoplast preparations. (A) Average R-luc and F-luc luminescence values for both DEX- and OHT-inducible beta plasmids (circles), for different days. Lines represent linear fits. (B) F-luc and R-luc coefficient of variation from beta plasmids, without (Raw) and with (TP Norm) normalization by the total protein content in the well. (C, D) Histogram of all basal (i.e., with no added inducer) F-luc luminescence values, plotted as RLU/(area*sec) (C) and on a log scale (D) for DEX-based plasmids. (E, F) Histogram of all basal (i.e., with no added inducer) F-luc luminescence values, plotted as RLU/(area*sec) (E) and on a log scale (F) for OHT-based plasmids.

FIG. 4A-E depicts graphs of data from testing alpha normalization with simulated and experimental repressor-repressible promoter data. (A) Coefficient of variation (COV) of the estimated parameter B with increasing noise levels in the distribution of the random multiplicative factor α. Non-normalized (Raw) data shows increasing COV, but the normalized data (Norm) is able to adjust for the increase in noise in a, and shows no significant change in the COV. (B) COV of the estimated parameter H also increases with increasing noise for the raw data fits, but stays approximately constant for the normalized data. (C) COV of the estimates for n do not show a difference between the normalized and raw data. (D, E) COV of experimental F-luc luminescence values for different inducer levels, in DEX-based plasmids (D) and in OHT-based plasmids (E). The COV of normalized data (Norm) is significantly reduced and more similar across inducer levels.

FIG. 5A-F depicts graphs of representative data and curve fits of some of the best performing promoter-repressor pairs. These six promoter repressor pairs were among those that satisfied all the established criteria for a functional pair, i.e., luminescence above the threshold, fold change greater than 1.3, Hill coefficient between biologically reasonable limits, and p-values of the fitted Hill coefficient n that were significantly smaller than 0.1.

FIG. 6A-E depicts graphs of data showing the validation of protoplast results through stable transformations in plants. (A) Protoplast cell counts at the time of luciferase imaging from the transient assay (Trans) and from the stable transformation (Stable), for three replicates. (B) F-luc and R-luc luminescence values from stable transformants or transient assays of the same replicates shown in (A). (C) Estimates of the promoter strength parameter B for the stably transformed plants (Plant) and transient expression (Trans) in protoplasts for three promoter-repressor pairs. (D) Estimates of the half-maximal repressor expression H, and (E) estimates of the Hill coefficient n, for the same three pairs.

FIG. 7A-F depicts plasmids used to test repressors, repressible promoters, and repressor-promoter combinations in transient protoplast assays. (A) Repressor module used to assemble synthetic repressors under control of dexamethasone (DEX). (B) Repressor module used to assemble synthetic repressors under control of 4-hydroxytamoxifen (OHT). BsaI restriction enzyme sites were included to exchange repressors. (C) Sub-cloning plasmid used to generate synthetic repressible promoters containing repressor binding sites upstream (BsaI/HindIII) or downstream (MluI/AatII) of the promoter. (D) Promoter module used to assemble repressible promoters controlling expression of the reporter gene, Renilla luciferase (R-luc). BsaI restriction enzyme sites were included to exchange promoters. A PEST protein degradation sequence was added to R-LUC to increase protein turnover and facilitate quantitative measurements of promoter repression. (E) Test plasmid used to assemble all DEX-inducible repressor-promoter combinations. (F) Test plasmid used to assemble all OHT-inducible repressor-promoter combinations. Repressors and repressible promoters were cloned into the test plasmids using KpnI restriction site. Both plasmids contain Firefly luciferase (F-luc) expressed under control of one of two inducible promoters, pOp6 and 10×N1. F-luc is used as a proxy for the amount of repressors produced in the system. LhGR2, DEX-activated transcription factor; NEV, OHT-activated transcription factor; NOST, nopaline synthase terminator; E9T, pea rbcS-E9 terminator; TB, transcription block; 35S, Cauliflower Mosaic Virus 35S promoter; AmpR, ampicillin resistance gene for bacterial selection; KanR, kanamycin resistance gene for bacterial selection; ColE1, origin of replication.

FIG. 8A-F depicts graphs of representative Fits to un-normalized data. A-F show the raw F-luc and R-luc luminescence values for six different repressor-promoter combinations. Solid lines are fits to Hill function forms using the nonlinear least squares fitting package in MATLAB. Open circles represent experimental data. (A) DEX 8×lexAspacer10nos EAR; (B) DEX 35s4×lexATATA OFPx, (C) OHT 2×LexAnos EAR; (D) DEX 8×LexAspacer10FMV EAR; (E) DEX 2×ga1435s EAR; (F) OHT 2×Gal4FMV EAR.

FIG. 9A-D depicts camera Correction Data. (A) The camera collects 1 image (i.e., frame) every 30th of a second. Each image represents the sum of pixel intensity within each well for every frame. Upper graph shows the F-luc signal is stable over time; lower graph shows the R-luc signal decays over the same time. (B) Representative graph showing the distribution of luciferase pixel intensity values RLU/(area*s) for each well for both a plate imaged with well position A1 in the top left hand corner of the camera (blue) and the same plate with A1 in the bottom left hand corner of the camera (green). In this experiment, each well should have approximately the same luminescence irrespective of its position on the plate. The luminescence value should not change on rotation of the plate. However, the data show that luminescence depends on well position and changes on plate rotation. (C) Representative images of the luminescence of individual wells for one of the 96-well plate experiments. Wells at the edges of the plate (blue boxes) show “new-moon-shaped” occluded areas, whereas wells at the center of the plate (green box) do not have these same occluded areas. (D) The percent change in the luminescence of the wells after rotation of the plate is shown for the original data (blue) and after imaging correction (green). The imaging correction removes almost all of the positional bias in the data.

FIG. 10A-C depicts schematic geometric diagram of imaging correction method. (A) Side view of the optical system and the well in microplate of interest. Part of the well is blocked from the sight of camera by the nontransparent wall. (B) Top view of the well of interest with the upper rim shifted to the bottom along the sight direction shown in (A). The overlapping area of the two cycles O₁ and O₂ is the visible part of the well bottom. (C) Side view of well in microplate as part of an imaginary cone. r, radius; h, height; d, diameter; a, area.

FIG. 11A-C depicts data generated by testing the normalization factor λᵢ* with simulated data. Blue bars are estimated parameter values of normalized data and red bars are of raw data. (A) The mean levels of estimated parameter B with increasing absolute levels in both mean and standard deviation of random number Nᵢⱼ. The raw data show decreases in mean values, but the normalized data show insensitivity to changes in absolute levels. (B) The mean levels of estimated parameter H also show decreases in raw data and remain constant with increasing absolute levels. (C) The mean levels of estimated parameter values of n do not show a difference between the normalized and raw data and across different absolute values.

FIG. 12A-F depicts graphs showing bootstrap results. (A-C) Distribution of parameter values (A) B, (B) H, and (C) n obtained from bootstrapping fits. (D) Comparison of bootstrapped estimates of the parameter B for the stably transformed plants and transient expression in protoplasts for three promoter-repressor pairs. (E). Comparison of bootstrapped estimates of half-maximal expression H. (F) bootstrapped estimates of the Hill coefficient n are shown for the same three cases.

### DETAILED DESCRIPTION

The present disclosure provides synthetic promoters and their corresponding synthetic repressor proteins. These promoters allow continuous gene expression in plants and plant cells in the absence of the corresponding repressor. Expression of genes operably linked to the synthetic promoters can be repressed by the presence of the corresponding repressor protein.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “construct” refers to any recombinant polynucleotide molecule.

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-0-methyl nucleotides, locked nucleic acids (LNA), peptide nucleic acids (PNA), and morpholinos.

The term “operably-linked”, as used herein, means that expression of a gene is under the control of a promoter with which it is spatially connected. A promoter may be positioned 5′ (upstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The terms “polypeptide” and “protein” are used interchangeably to refer to a polymer of amino acid residues.

The term “promoter”, as used herein, refers to a synthetic or naturally-derived nucleic acid sequence which is capable of conferring, activating or enhancing expression of a gene in a cell. A promoter may comprise one or more specific transcriptional regulatory sequences to activate or enhance expression and/or to alter the spatial expression and/or temporal expression of the gene. In prokaryotes, these regulatory elements may include, but are not limited to, the −35 RNA polymerase recognition sequence, the −10 ribosome binding sequence, and upstream distal enhancer elements, which can be located as much as several thousand base pairs from the start site of transcription. Non-limiting examples of regulatory elements found in eukaryotes include the TATA box, initiator elements, downstream core promoter element, CAAT box, and the GC box. An inducible promoter is a promoter that is induced by the presence or absence of biotic or abiotic factors. An inducible promoter allows for the expression of the gene operably linked to it to be turned on or off (i.e., controlled).

The term “specifically binds”, as used herein in reference to the interaction of DNA-binding molecule and its cognate binding element, means that the interaction is dependent upon the presence of a particular structure. For example, specific binding between a DNA-binding molecule and a synthetic repressible construct may be demonstrated, for example, by the absence of binding of the DNA-binding domain to the synthetic repressible promoter construct when the binding element(s) are removed.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

### II. Constructs

The present disclosure provides synthetic repressor constructs and synthetic repressor encoded thereby, as well as synthetic repressible promoter constructs for use in combination with the synthetic repressor constructs/synthetic repressors.

In various embodiments, the constructs of the present disclosure can be present in a vector. Suitable vectors include plasmid vectors, phagemids, cosmids, artificial/mini-chromosomes, transposons, and viral vectors (e.g., lentiviral vectors, adeno-associated viral vectors, etc.). The choice of the vector will vary depending upon the intended use (e.g., stable transformation in bacterial cells, stable transformation in plant cells, transient transformation in plant cells, etc.). In one embodiment, the synthetic repressor construct and the synthetic repressible promoter construct are present in a plasmid vector. In another embodiment, the synthetic repressor construct is present in first plasmid vector and the synthetic repressible promoter construct is present in a second plasmid vector. Non-limiting examples of suitable plasmid vectors include pUC, pBR322, pET, pBluescript, pCAMBIA2300, pRI 101, pBI121, pPZP100, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

(a) Synthetic Repressor Construct and Synthetic Repressor Protein

In an aspect, the present disclosure provides a synthetic repressor construct for modifying gene expression in a plant. The synthetic repressor construct comprises a nucleic acid encoding a transcriptional repressor domain linked to a DNA-binding domain. The transcriptional repressor domain and the DNA-binding domain are operable (i.e., carry out their intended function) in the same plant species but may or may not be derived from the same source and may or may not be native to the plant species. The nucleic acid encoding the transcriptional repressor domain can be linked directly to the DNA-binding domain or indirectly to the DNA-binding domain (e.g., separated by three or more nucleotides). The polynucleotide sequence encoding the transcriptional repressor domain can be 5′ of the polynucleotide sequence encoding the DNA-binding domain, or vice versa. In certain embodiments, the nucleic acid encoding a transcriptional repressor domain linked to a DNA-binding domain further encodes a nuclear localization signal. In all embodiments, the polynucleotide sequence encoding the transcriptional repressor domain and the polynucleotide sequence encoding the DNA-binding domain are in-frame such that when a promoter is operably linked to a synthetic repressor construct, the expressed nucleic acid will be translated as single protein (e.g., a fusion protein).

In another aspect, the present disclosure provides a synthetic repressor comprising a transcriptional repressor domain and a DNA-binding domain. The transcriptional repressor domain can be linked directly to the DNA-binding domain or indirectly to the DNA-binding domain. In embodiments where the transcriptional repressor domain and the DNA-binding domain are indirectly linked, the domains are connected by a flexible linker comprised of one or more amino acids (e.g., 1, 2, 3, 4, 5, 6, 7, 8, 9, 10, or more amino acids). In certain embodiments, the flexible linker can be a nuclear localization signal and/or a marker domain.

Transcriptional repressor domains are protein domains that are sufficient to confer the capacity for repression of transcription when linked to a heterologous DNA-binding domain. In general, a transcriptional repressor domain interacts with transcriptional control elements and/or transcriptional regulatory proteins (e.g., transcription factors, RNA polymerases, etc.) to decrease and/or terminate transcription of a gene. Non-limiting examples of transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, MeCP2, BRD transcriptional repressor domains, OVATE family protein (OFP) transcriptional repressor domains, mSin interaction domains (SID), EAR transcriptional repressor domains (including SRDX domains), and derivatives thereof.

A DNA-binding domain is an independently folded protein domain that contains at least one structural motif that recognizes a double- or single-stranded DNA. Non-limiting examples of suitable DNA-binding domains include a helix-turn-helix motif, a zinc finger domain, a leucine zipper domain, a winged helix domain, a winged helix-turn-helix domain, a helix-loop-helix domain, an HGM-box, a WOr3 domain, an OB-fold domain, a B3 DNA binding domain, or a transcriptional activator-like effectors (TALE) DNA binding domain. Suitable DNA-binding domains can also be computationally designed. See, for example, Huang, et al., (2016a) Nature, 537: 320-327; Huang, et al. (2016b) Nat Chem Biol, 12: 29-34; Rose, et al. (2017) Nat Chem Biol, 13: 119-12. DNA-binding domains can recognize a specific DNA sequence (i.e., a recognition sequence) or have a general affinity to DNA, though sequence-specific DNA binding domains are preferred. Suitable DNA-binding domains may be derived from any known DNA-binding protein, including but not limited to viral, prokaryotic or eukaryotic transcription factors or endonucleases. In various embodiments, a suitable DNA-binding domain may be derived from a plant DNA-binding protein, an insect DNA-binding protein, a yeast DNA-binding protein, a fungal DNA-binding protein, a bacterial DNA-binding protein, a nematode DNA-binding protein a mammalian DNA-binding protein. Alternatively, a suitable DNA-binding domain may be any other type of sequence-specific repressor known in the art including, but not limited to a CRISPR/cas protein lacking endonuclease activity. For example, Cas9 can be modified by mutating the RuvC and HNH domains such that they no longer possess nuclease activity. While CRISPR/cas proteins themselves do not recognize double- or single-stranded DNA, they can be used to target any DNA sequence with the help of guide-RNAs, which have sequence homology to the target site. Methods for designing DNA-binding domains and other sequence-specific repressors to target various sites are known in the art.

The present disclosure contemplates various combinations of transcriptional repressor domains and DNA-binding domains known in the art, provided the transcriptional repressor domain and the DNA-binding domain are both operable in the same plant species. In certain embodiments, the DNA-binding domain is not native to the plant species in which expression is desired. In further embodiments, the DNA-binding domain is a sequence-specific DNA binding domain. In still further embodiments, the DNA binding domain is a sequence-specific, DNA-binding domain with a recognition sequence that is not specifically recognized by a transcription factor native to the plant species in which expression is desired.

In an exemplary embodiment, a synthetic repressor construct comprises a nucleic acid encoding a transcriptional repressor domain linked to a DNA-binding domain, wherein the DNA-binding domain is a yeast Gal4 DNA-binding domain or a bacterial LexA DNA-binding domain, and the transcriptional repressor domain is an EAR transcriptional repressor domain, an OFP transcriptional repressor domain, or a BRD transcriptional repressor domain. In a further embodiment, the OFP transcriptional repressor domain is SEQ ID NO: 35. In another exemplary embodiment, a synthetic repressor construct comprises a nucleic acid encoding a transcriptional repressor domain linked to a DNA-binding domain, wherein the transcriptional repressor domain is an OFP transcriptional repressor domain and the DNA-binding domain is a bacterial LexA DNA-binding domain. In a further embodiment, the OFP transcriptional repressor domain is SEQ ID NO: 35. In another exemplary embodiment, a synthetic repressor construct comprises a nucleic acid encoding a transcriptional repressor domain linked to a DNA-binding domain, wherein the transcriptional repressor domain is a BRD transcriptional repressor domain and the DNA-binding domain is a yeast Gal4 DNA-binding domain. In another exemplary embodiment, a synthetic repressor construct comprises a nucleic acid selected from the group consisting of SEQ ID NO: 27-34.

Synthetic repressor constructs of the present disclosure may further comprise a polynucleotide encoding additional domains and/or other genetic elements. Additional domains include, but are not limited to, nuclear localization signal or other signal sequences for targeting proteins to subcellular compartments, a cell-penetrating domain, and a marker domain. Other genetic elements include, but not limited to, promoter sequences, terminators, translational regulatory sequences such as ribosome binding sites and internal ribosome entry sites, enhancers, silencers, insulators or other boundary elements, polyadenylation signals, locus control regions, etc.

In some embodiments, a promoter is operably linked to a synthetic repressor construct of the present disclosure. The promoter can be a constitutive promoter or an inducible promoter, and can be a synthetic promoter or a promoter from a native gene (e.g. a viral promoter, a bacterial promoter, an archaeal promoter, a plant promoter, an insect promoter, a nematode promoter, a yeast promoter, a fungal promoter, a mammalian promoter, etc.). In certain embodiments, the promoter is capable of conferring expression in a plant species. In further embodiments, the promoter is an inducible promoter. In still further embodiments, the promoter is sensitive to a computationally designed ligand binding transcription factor. See, for example, Bick, et al., (2017) eLife, 6; Feng, et al., (2015) eLife, 4, e10606.

Any inducible promoter for plants can be utilized in the instant invention. The promoter may be responsive to an internal or an external factor (inducer). Several systems for induction of transgene expression in plants are known in the art. See, for example, Borghi et al, Method Mol Biol, 2010, 655:65-75; Gatz Curr Opinion Biotechnology 1996, 7:168-172; U.S. Pat. No. 5,750,385, U.S. Pat. No. 5,420,034, U.S. Pat. No. 5,753,475, U.S. Pat. No. 6,281,410, each hereby incorporated by reference in its entirety. Additional, non-limiting examples include AlcR/AlcA (ethanol inducible); GR fusions, GVG, and pOp/LhGR (dexamethasone inducible); XVE/OlexA (beta-estradiol inducible); as well as known promoters responsive to a variety of environmental factors, including but not limited to light-inducible or stress-inducible (e.g. water deficit, cold, heat, salt, pest, disease, nutrient stress, etc.) promoters. Suitable inducible promoters are also described in the examples.

When expression of a synthetic repressor of the present disclosure is desired in a eukaryotic cell (e.g., in an isolated plant cell, a cell of a plant seed, or a cell in a whole plant), the synthetic repressor also comprises a nuclear localization signal. In general, a nuclear localization signal comprises a stretch of basic amino acids. Nuclear localization signals are known in the art. The nuclear localization signal can be located at the N-terminus, the C-terminus, or in an internal location of the synthetic repressor.

Transport of protein produced by transgenes to a subcellular compartment such as the chloroplast, vacuole, peroxisome, glyoxysome, cell wall or mitochondrion, or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Any signal sequence known in the art is contemplated by the present invention.

In still other embodiments, the synthetic repressor can further comprise at least one marker domain. Non-limiting examples of marker domains include fluorescent proteins, luciferase enzymes, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl,), blue fluorescent proteins (e.g., EBFP, EBFP2, Azurite, mKalamal, GFPuv, Sapphire, T-sapphire,), cyan fluorescent proteins (e.g. ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato) or any other suitable fluorescent protein. In other embodiments, the marker domain can be a luciferase enzyme. Non-limiting examples include firefly luciferase, Renilla luciferase, Nanoluc luciferase, and derivatives thereof. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin. The marker domain can be located at the N-terminus, the C-terminal, or in an internal location of the synthetic repressor.

In still other embodiments, the synthetic repressor can further comprise at least one cell penetrating domain. In one embodiment, the cell-penetrating domain can be a cell-penetrating peptide sequence derived from the HIV-1 TAT protein. In another embodiment, the cell-penetrating domain can be TLM, a cell-penetrating peptide sequence derived from the human hepatitis B virus. In still another embodiment, the cell-penetrating domain can be MPG. In an additional embodiment, the cell-penetrating domain can be Pep-1, VP22, a cell penetrating peptide from Herpes simplex virus, or a polyarginine peptide sequence. The cell-penetrating domain can be located at the N-terminus, the C-terminus, or in an internal location of the synthetic repressor.

(b) Synthetic Repressible Promoter Construct

In another aspect, the present disclosure provides a synthetic repressible promoter construct for use in combination with a synthetic repressor construct of Section I(a). In various embodiments, the 3′ end of a synthetic repressible promoter construct described below may be proximal to a cloning site. Alternatively, or in addition, a synthetic repressible promoter construct described below may be operably linked to a nucleic acid encoding a protein of interest.

Synthetic repressible promoter constructs of the present disclosure comprise (i) a nucleic acid sequence encoding a core promoter region capable of conferring constitutive gene expression in a plant species, the promoter region optionally comprising a TATA box, and (ii) a synthetic regulatory element comprising at least one copy of a binding element having a nucleic acid sequence capable of specifically binding the DNA binding domain of the cognate synthetic repressor. In some embodiments, a synthetic repressible promoter construct is operably linked to a nucleic acid encoding a protein of interest. In preferred embodiments, the binding element is a recognition sequence and the DNA-binding protein is a sequence-specific, DNA-binding protein.

A core promoter is typically a minimal set of nucleotides capable of driving accurate transcription initiation when bound by a basal transcription factor. A core promoter may contain a TATA-box, a GA element, a CAAT box, or other core promoter elements known in the art. Suitable core promoters are operable in the plant species in which expression is desired, but may or may not be native to the plant species. Further, the core promoter may or may not be derived from the same species as the transcriptional repressor domain, DNA-binding domain, or any other element of the synthetic repressor construct; however, in order to function together each of these elements must be operable in the plant species in which expression is desired.

Suitable core promoters capable of conferring constitutive gene expression in a desired plant species are known in the art. Non-limiting examples of suitable core promoters include promoters from plant viruses (e.g., Cauliflower Mosaic Virus (CaMV 35S) promoter, Figwort Mosaic Virus (FMV) promoter, etc.) bacterial plant pathogens (e.g., Nopaline Synthase (NOS) promoter, etc.), the promoters from such genes as rice actin (e.g., OsACT2.1), maize ubiquitin (e.g., ZmUBI1), and corn H3 histone, and also the ALS promoter, a XbaI/NcoI fragment 5′ to the Brassica napus ALS3 structural gene (or a nucleotide sequence that has substantial sequence similarity to the XbaI/NcoI fragment). In some embodiments, the core promoter confers constitutive gene expression only in one tissue or cell type of the plant. In other embodiments, the core promoter confers constitutive gene expression in one or more cell types of the plant. In other embodiments, the core promoter confers constitutive gene expression in one or more tissues of the plant. In other embodiments, the core promoter confers constitutive gene expression in all cell types of the plant.

Any tissue-specific or tissue-preferred promoter can be utilized in the instant invention. Exemplary tissue-specific or tissue-preferred promoters include, but are not limited to, a seed-preferred promoter such as that from the phaseolin gene; a leaf-specific and light-induced promoter such as that from cab or rubisco; an anther-specific promoter such as that from LAT52; a pollen specific promoter such as that from Zm13 or a microspore-preferred promoter such as that from apg.

At a position upstream of the core promoter, downstream of the core promoter but before a translational start site for a protein of interest (e.g., within a 5′ UTR, etc.) or a cloning site, proximal to the 5′ end of the optionally present TATA box when present, or any combination thereof, a repressible promoter of the present disclosure comprises at least one copy of a binding element having a nucleic acid sequence capable of specifically binding the DNA binding domain of its cognate synthetic repressor. While binding elements are placed proximal to the 5′ end of the promoter in various embodiments disclosed in the Examples, the present disclosure contemplates one or more binding elements at various positions upstream of a core promoter at greater distances (e.g., about 20 bp, 40 bp, 60 bp, 80 bp, 100 bp or more). The presence of one of more binding elements creates a repressible promoter from the constitutive core promoter. Tunable expression from the promoter can be achieved by independently varying the number of different binding elements, the copy number of a binding element at a given position, and/or the spacing between binding elements at a given position.

In some embodiments, a synthetic repressor promoter construct may comprise 2 or more different types of binding elements. For example, a synthetic repressor promoter construct may comprise 2, 3, 4, 5, 6, 7, 8, 9, 10, or more different types of binding elements. In certain embodiments, a synthetic repressor promoter construct may comprise 2 or more recognition sequences. Alternatively or in addition, a synthetic repressor promoter construct may comprise 2 or more copies of any given binding element. For example, a synthetic repressor promoter construct may comprise 2, 3, 4, 5, 6, 7, 8, 9, 10, or more copies of a binding element.

The total number of binding elements in a synthetic repressor construct can and will vary depending upon the level of repression desired and the type of binding element(s). Generally speaking, increasing the number of binding elements will result in greater repression. However, the number of binding elements should not substantially weaken the constitutive activity of the core promoter in the absence of repressor. For illustrative purposes only, if about 6-10 copies of a binding element that is 20 nucleotides in length begins to adversely weaken a core promoter in the absence of a repressor, then a similar effect may be seen with about 3-5 copies of a binding element that is 40 nucleotides in length. In some embodiments, a synthetic repressor promoter construct may comprise at least 2 and no more than 10 copies of all binding elements. In still other embodiments, a synthetic repressor promoter construct may comprise at least 2 and no more than 8 copies of all binding elements. In still other embodiments, a synthetic repressor promoter construct may comprise at least 2 and no more than 6 copies of all binding elements. In each of the above embodiments, the binding elements may be the same or different.

In embodiments that comprise 2 or more copies of a binding element, or 2 or more different binding elements, the binding elements may be at the same position or at different positions. As a non-limiting example, if a synthetic repressor promoter construct comprises a core promoter, a TATA box and 2 copies of a binding element, then (1) both copies may be at a single position selected from upstream of the core promoter, downstream of the core promoter but before a translational start site for the protein of interest/cloning site, or proximal to the 5′ end of the TATA box; (2) one copy may be upstream of the core promoter and one copy may be downstream of the core promoter but before a translational start site for the protein of interest/cloning site; (3) one copy may be upstream of the core promoter and one copy may be proximal to the 5′ end of the TATA box; or (4) one copy may be downstream of the core promoter but before a translational start site for the protein of interest/cloning site and one copy may be proximal to the 5′ end of the TATA box. As another non-limiting example, if a synthetic repressor promoter construct comprises a core promoter, a TATA box and 1 copy each of two different binding elements, then (1) both binding elements may be at a single position selected from upstream of the core promoter, downstream of the core promoter but before a translational start site for the protein of interest/cloning site, or proximal to the 5′ end of the TATA box; (2) one binding element may be upstream of the core promoter and one binding element may be downstream of the core promoter but before a translational start site for the protein of interest/cloning site; (3) one binding element may be upstream of the core promoter and one binding element may be proximal to the 5′ end of the TATA box; or (4) one binding element may be downstream of the core promoter but before a translational start site for the protein of interest/cloning site and one binding element may be proximal to the 5′ end of the TATA box.

Alternatively or in addition to the above, in embodiments that comprise 2 or more copies of a binding element at any one position (or 2 or more different binding elements at any one position), the binding elements may be separated from each other by a nucleic acid spacer sequence of about 2 to about 20 nucleotides, preferably about 2 to about 10 nucleotides.

In an exemplary embodiment, a synthetic repressible promoter construct comprises (a) a nucleic acid sequence encoding a core promoter region capable of conferring constitutive gene expression in a plant species, wherein the core promoter region is selected from the group consisting of Cauliflower Mosaic Virus (CaMV35S) promoter, Figwort Mosaic Virus (FMV) promoter, Nopaline Synthase (NOS) promoter, Ubiquitin-1 promoter from maize (ZmUBI1), and Actin 2.1 promoter from rice (OsACT2.1); and (b) a synthetic regulatory element comprising at least two copies of recognition sequence for a bacterial LexA DNA-binding domain or at least two copies of a recognition sequence for a yeast Gal4 DNA-binding domain, wherein the two or more copies of the recognition sequence is inserted at a position upstream of the core promoter, downstream of the core promoter but before a translation start site for a protein of interest or a cloning site, or proximal to the 5′ end of the optionally present TATA box when present. In another exemplary embodiment, a synthetic repressor construct comprises a nucleic acid selected from the group consisting of SEQ ID NO: 27-34.

Synthetic repressible promoter constructs of the present disclosure may further comprise a polynucleotide encoding additional genetic elements including, but not limited to, terminators, translational regulatory sequences such as ribosome binding sites and internal ribosome entry sites, enhancers, silencers, insulators or other boundary elements, polyadenylation signals, locus control regions, etc.

(c) Synthetic Genetic Circuit

In another aspect, the present disclosure provides a synthetic genetic circuit for modifying expression of a protein of interest in a plant cell. A genetic circuit, as used herein, refers to a set of expression cassettes (genes) that interact to produce an output. The output of a genetic circuit is typically controlled, or “tuned”, through inducible transcription factors and cis-regulatory elements. For example, in operation, an input signal may trigger the expression of, or otherwise alter the generation of, a product from a first expression cassette. The first product may then directly or indirectly alter the expression of a product encoded for by another part of the genetic circuit (e.g., a second expression cassette).

The synthetic genetic circuit of the present disclosure comprises: (a) a nucleic acid construct comprising a nucleic acid encoding a protein of interest; (b) a synthetic repressible promoter construct operably linked to the nucleic acid encoding the protein of interest; and (c) a promoter operably linked to a synthetic repressor construct. Suitable synthetic repressor constructs are described in Section I(a) and suitable synthetic repressible promoter constructs are described in Section I(b). In all embodiments, the transcriptional repressor domain and the DNA-binding domain of the synthetic repressor are operable in the same plant species as the promoter operably linked to the synthetic repressor construct and the core promoter of the synthetic repressible construct.

By assembling genetic circuits as detailed herein, a range of quantitatively distinct responses can be produced. How well a synthetic repressor actually works on a quantitative level is measurable and allows for the assembling of components into digital-like systems. For example, using synthetic repressors allows one to produce a NOT gate. NOT gates can be combined to produce NOR gates. NOR gates are Boolean complete, meaning that any computational function can be designed (e.g. any LOGIC function found in electronic circuits, i.e., design circuit boards). For example, see, Brophy et al. (2014) Nat Methods, 11: 508-520; Moon et al. (2012) Nature, 491:249-253; Tasmir et al. (2011) Nature, 469: 212-215.

In an exemplary embodiment, the transcriptional repressor domain of the synthetic repressor is an OFP transcriptional repressor domain and the DNA-binding domain of the synthetic repressor is a bacterial LexA DNA-binding domain. In further embodiments, the core promoter is cauliflower mosaic virus 35S promoter. In still further embodiments, the OFP transcriptional repressor domain is SEQ ID NO: 35.

In another exemplary embodiment, the transcriptional repressor domain of the synthetic repressor is a BRD transcriptional repressor domain and the DNA-binding domain of the synthetic repressor is a yeast Gal4 DNA-binding domain.

In each of the above embodiments, the promoter operably linked to the synthetic repressor construct can be a constitutive promoter or, preferably, an inducible promoter. In embodiments where an inducible promoter is operably linked to a synthetic repressor construct, the synthetic repressor produced therefrom is conditionally expressed such that the repressor protein is only be available to the synthetic repressible promoter under certain conditions and, therefore, drive expression of the protein of interest under those same conditions. Suitable inducible promoters are described in Section I(a). In some embodiments, the inducible promoter confers gene expression only in one tissue or cell type of the plant. In other embodiments, the inducible promoter confers gene expression in one or more cell types of the plant. In other embodiments, the inducible promoter confers gene expression in one or more tissues of the plant. In other embodiments, the inducible promoter confers gene expression in all cell types of the plant.

Any tissue-specific or tissue-preferred promoter can be utilized in the instant invention. Exemplary tissue-specific or tissue-preferred promoters include, but are not limited to, a seed-preferred promoter such as that from the phaseolin gene; a leaf-specific and light-induced promoter such as that from cab or rubisco; an anther-specific promoter such as that from LAT52; a pollen specific promoter such as that from Zm13 or a microspore-preferred promoter such as that from apg.

In further embodiments, the protein of interest negatively regulates the inducible promoter operably linked to the synthetic repressor construct.

Transport of protein produced by transgenes to a subcellular compartment such as the chloroplast, vacuole, peroxisome, glyoxysome, cell wall or mitochondrion, or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Any signal sequence known in the art is contemplated by the present invention.

### III. Kits

A further aspect of the present disclosure encompasses kits comprising any one of the constructs, or synthetic genetic circuits, detailed above.

In some embodiments, a kit comprises a synthetic repressor construct of Section I(a) and/or a synthetic repressible promoter construct of Section I(b).

In other embodiments, a kit comprises a vector comprising a promoter operably linked to a synthetic repressor construct of Section I(a) and/or a vector comprising a synthetic repressible promoter construct of Section I(b) and cloning site proximal to the 3′ end of a synthetic repressible promoter construct.

In other embodiments, a kit comprises a vector comprising (a) a promoter operably linked to a synthetic repressor construct of Section I(a) and (b) a synthetic repressible promoter construct of Section I(b) and cloning site proximal to the 3′ end of a synthetic repressible promoter construct.

In other embodiments, a kit comprises a plurality of synthetic genetic circuits of Section I(c), wherein each synthetic genetic circuit of the kit varies from the other synthetic genetic circuits in the number of binding elements at a given position and/or the spacing between the binding elements at a given position.

In each of the above embodiments, a kit can further comprise cells competent for transformation or transfection, transformation or transfection reagents, restriction enzymes, inducers, buffers, and the like.

In further embodiments, a kit can also comprise a construct comprising an inducible promoter operably linked to a reporter. The inducible promoter can be same as, or different than, the inducible promoter operably linked to the synthetic repressor construct. Non-limiting examples of suitable reporters include fluorescent proteins, purification tags, epitope tags, and the like. Exemplary fluorescent proteins, purification tags, epitope tags are described in Section I(a).

### IV. Transgenic Plants

A further aspect of the present disclosure encompasses a transgenic plant cell comprising a synthetic repressor construct of Section I(a). In another aspect, the present disclosure provides a transgenic plant cell comprising a synthetic repressible promoter construct of Section I(b). In another aspect, the present disclosure provides a transgenic plant cell comprising a synthetic genetic circuit of Section I(c). The plant cell can been an isolated plant cell, a cell in a whole plant, or cell in plant structure (e.g., seed, flower, fruit, etc.). In various embodiments, the plant cell can be a parenchyma cell, a collenchyma cell, a sclerenchyma cell, a xylem cell, or a phloem cell.

In each of the above aspects, the transgenic plant cell can be derived from a monocot or a dicot. In certain embodiments, the plant cell is crop plant cell. Non-limiting examples of crop plants include grain crops (e.g., rice, Jowar, wheat, maize, barley, millets, etc.), pulse/legume crops (e.g., green gram, black gram, soybean, pea, cowpea, etc.), oil seed crops (e.g., groundut, mustard, sunflower, sesamum, linseed, etc.), forage crops (e.g., fickler, hay, silage, etc.), fiber crops (e.g., cotton, steam, jute, mesta, sun hemp, etc.), root crops (e.g., sugar beet, carrots, turnips, etc.), tuber crops (e.g., potato, yam, etc.), sugar crops (e.g., sugarcane, sugar beet, etc.), vegetable crops, green manure crops, medicinal and aromatic crops (e.g., cinchona, isabgoli, opium poppy, senna, belladonna, rauwolfra, iycorice, lemon grass, citronella grass, palmorsa, Japanese mint, peppermint, rose geranicem, jasmine, henna etc.).

In embodiments encompassing whole plants or plant structures, one or more of the cell types may comprise a synthetic repressor construct of Section I(a), a synthetic repressible promoter construct of Section I(b), or The synthetic genetic circuit of Section I(c). In other embodiments encompassing whole plants or plant structures, one or more of the tissue types may be comprised of a transgenic plant cell comprising a synthetic repressor construct of Section I(a), a synthetic repressible promoter construct of Section I(b), or The synthetic genetic circuit of Section I(c).

Methods for transiently or stably transforming plant cells are well known in the art. For example, numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, inc., Boca Raton, 1993) pages 89-119. Additional methods are further detailed in the examples.

Synthetic genetic circuits disclosed herein can be used to engineer transgenic plants that express various phenotypes of agronomic interest, or to engineer transgenic plants for recombinant protein production.

In some embodiments, the nucleic acid encoding a protein of interest is a gene that confers resistance to pests or disease including, but not limited to, those that encode: (a) plant disease resistance genes; (b) a lectin; (c) a vitamin-binding protein; (d) an enzyme inhibitor; (e) an insect-specific hormone or pheromone, mimetic based thereon, antagonist or agonist thereof; (f) an insect-specific peptide or neuropeptide which, upon expression, disrupts the physiology of the affected pest; (g) an insect-specific venom produced in nature by a snake, a wasp, etc.; (h) an enzyme responsible for a hyperaccumulation of a monterpene, a sesquiterpene, a steroid, hydroxamic acid, a phenylpropanoid derivative or another non-protein molecule with insecticidal activity; (i) an enzyme involved in the modification, including the post-translational modification, of a biologically active molecule, for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic; (j) a molecule that stimulates signal transduction; (k) a hydrophobic moment peptide; (l) a membrane permease, a channel former or a channel blocker; (m) a viral-invasive protein or a complex toxin derived therefrom; (n) an insect-specific antibody or an immunotoxin derived therefrom; (o) a virus-specific antibody; (p) a developmental-arrestive protein produced in nature by a pathogen or a parasite; or (q) a developmental-arrestive protein produced in nature by a plant.

In other embodiments, the nucleic acid encoding a protein of interest is a gene that confers resistance to herbicides including, but not limited to: (a) a herbicide that inhibits the growing point or meristem, such as an imidazalinone or a sulfonylurea; (b) glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes); or (c) a herbicide that inhibits photosynthesis, such as a triazine (psbA and gs+ genes) and a benzonitrile (nitrilase gene).

In other embodiments, the nucleic acid encoding a protein of interest is a gene that confers or contributes to a value-added trait including, but not limited to (i) introduction of a phytase-encoding gene would enhance breakdown of phytate, adding more free phosphate to the transformed plant; (ii) a gene could be introduced that reduces phytate content; or (iii) modified carbohydrate composition effected, for example, by transforming plants with a gene coding for an enzyme that alters the branching pattern of starch.

In other embodiments, the nucleic acid encoding a protein of interest is a gene that encodes or produces a therapeutic such as an antibody, a hormone, etc.

### V. Methods

In another aspect, the present disclosure provides a method for modifying expression of a protein of interest in a plant. The method comprises introducing a synthetic genetic circuit of Section I(c) into a cell of the plant, wherein the promoter operably linked to the synthetic repressor construct is an inducible promoter. In some embodiments, the method further comprises varying the level of expression of the protein of interest across different cell types or tissues types in the plant by varying the number of binding elements at a given position and/or the spacing between the 2 or more binding elements at a given position.

In another aspect, the present disclosure provides a method for creating a library comprising a plurality of synthetic repressible promoter constructs. The method comprises providing a construct comprising a core promoter capable of conferring constitutive gene expression in a plant species, wherein the promoter optionally comprises a TATA box, and modifying the construct a plurality of times by (i) introducing one or more copies of a binding element having a nucleic acid sequence capable of specifically binding a DNA-binding domain, the copy of the binding element inserted at a position upstream of the core promoter, downstream of the core promoter but before a translation start site for a protein of interest, or proximal to the 5′ end of the optionally present TATA box when present, and then (ii) varying the number of binding elements at a given position and/or the spacing between the 2 or more binding elements at a given position. In various embodiments, the core plant promoter is operably linked to a nucleic acid encoding a protein of interest. Alternatively, or in addition, the 3′ end of the core promoter can be proximal to a cloning site. In further embodiments, the construct comprising a core promoter capable of conferring constitutive gene expression in a plant species is provided in the form of a vector. In still further embodiments, the vector further comprises a synthetic repressor construct, the synthetic repressor construct comprising a promoter operably linked to a nucleic acid encoding a transcriptional repressor domain and a DNA-binding domain; wherein the DNA-binding domain specifically binds to a binding element of the synthetic repressible promoter construct, and wherein the transcriptional repressor domain of the synthetic repressor, the DNA-binding domain of the synthetic repressor, the promoter operably linked to the synthetic repressor construct, and the core promoter of the synthetic repressible construct are each operable in the same plant species.

In another aspect, the present disclosure provides a method for selecting in vitro a synthetic gene circuit for expression in a plant. Generally speaking the method comprises (a) introducing a plurality of synthetic genetic circuits into isolated plant cells to produce a plurality of isolated plant cells that have only one type of synthetic genetic circuit, each synthetic genetic circuit comprises (i) a nucleic acid construct comprising a nucleic acid encoding a first reporter; (ii) a synthetic repressible promoter construct operably linked to the nucleic acid encoding the first reporter; and (iii) a promoter operably linked to a synthetic repressor construct; (b) further introducing into each of the isolated plant cells a construct comprising an inducible promoter operably linked to a second reporter, wherein the inducible promoter of the construct is the same as the inducible promoter of the synthetic genetic circuits; (c) for each plant cell produced from step (b), adding inducer to an in vitro culture of the plant cell, culturing the plant cell for a sufficient amount of time, measuring the amount of each reporter, and determining a quantitative parameter of the synthetic genetic circuit; and (d) selecting a synthetic genetic circuit based on the quantitative parameter determined in step (c). In various embodiments, the order of steps (a) and (b) may be interchanged. Synthetic repressor constructs are detailed in Section I(a) and synthetic repressible promoter constructs are detailed in Section I(b). The transcriptional repressor domain and the DNA-binding domain of the synthetic repressor are operable in the same plant species as the promoter operably linked to the synthetic repressor construct and the core promoter of the synthetic repressible construct. Further, each synthetic genetic circuit varies from the other synthetic genetic circuits in the number of binding elements at a given position and/or the spacing between the binding elements at a given position.

Methods for extracting quantitative parameters of synthetic genetic circuits are known in the art, and further detailed in the examples.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

| SEQ ID | | |
|---|---|---|
| NO: | Name | DNAsequence |
| 1 | 2xGal4FMV | CGGAAGACTCTCCTCCGAGCGGAAGACTCTCCTCCGaagcttAGGAT |
| | | TTAGCAGCATTCCAGATTGGGTTCAATCAACAAGGTACGAGCCATA |
| | | TCACTTTATTCAAATTGGTATCGCCAAAACCAAGAAGGAACTCCCAT |
| | | CCTCAAAGGTTTGTAAGGAAGAATTCTCAGTCCAAAGCCTCAACAA |
| | | GGTCAGGGTACAGAGTCTCCAAACCATTAGCCAAAAGCTACAGGA |
| | | GATCAATGAAGAATCTTCAATCAAAGTAAACTACTGTTCCAGCACAT |
| | | GCATCATGGTCAGTAAGTTTCAGAAAAAGACATCCACCGAAGACTT |
| | | AAAGTTAGTGGGCATCTTTGAAAGTAATCTTGTCAACATCGAGCAG |
| | | CTGGCTTGTGGGGACCAGACAAAAAAGGAATGGTGCAGAATTGTTA |
| | | GGCGCACCTACCAAAAGCATCTTTGCCTTTATTGCAAAGATAAAGC |
| | | AGATTCCTCTAGTACAAGTGGGGAACAAAATAACGTGGAAAAGAGC |
| | | TGTCCTGACAGCCCACTCACTAATGCGTATGACGAACGCAGTGACG |
| | | ACCACAAAAGAATTCCCTCTATATAAGAAGGCATTCATTCCCATTTG |
| | | AAGGATCATCAGATACTCAACG |
| 2 | 2xGal4NOS | CGGAAGACTCTCCTCCGAGCGGAAGACTCTCCTCCGaagcttAGGCG |
| | | GGAAACGACAATCTGATCATGAGCGGAGAATTAAGGGAGTCACGTT |
| | | ATGACCCCCGCCGATGACGCGGGACAAGCCGTTTTACGTTTGGAA |
| | | CTGACAGAACCGCAACGATTGAAGGAGCCACTCAGCCGCGGGTTT |
| | | CTGGAGTTTAATGAGCTAAGCACATACGTCAGAAACCATTATTGCG |
| | | CGTTCAAAAGTCGCCTAAGGTCACTATCAGCTAGCAAATATTTCTTG |
| | | TCAAAAATGCTCCACTGACGTTCCATAAATTCCCCTCGGTATCCAAT |
| | | TAGAGTCTCATATTCACTCTCAATCCAAATAATCTGCACCG |
| 3 | 2xGal435S | CGGAAGACTCTCCTCCGAGCGGAAGACTCTCCTCCGaagcttTGAGA |
| | | CTTTTCAACAAAGGGTAATATCGGGAAACCTCCTCGGATTCCATTG |
| | | CCCAGCTATCTGTCACTTCATCAAAAGGACAGTAGAAAAGGAAGGT |
| | | GGCACCTACAAATGCCATCATTGCGATAAAGGAAAGGCTATCGTTC |
| | | AAGATGCCTCTGCCGACAGTGGTCCCAAAGATGGACCCCCACCCA |
| | | CGAGGAGCATCGTGGAAAAAGAAGACGTTCCAACCACGTCTTCAAA |
| | | GCAAGTGGATTGATGTGATATCTCCACTGACGTAAGGGATGACGCA |
| | | CAATCCCACTATCCTTCGCAAGACCCTTCCTCTATATAAGGAAGTTC |
| | | ATTTCATTTGGAGAGGA |
| 4 | 2xGal4sp1035S | CGGAAGACTCTCCTCCGTAATGCTTCGCGGAAGACTCTCCTCCGAA |
| | | GCTTTGAGACTTTTCAACAAAGGGTAATATCGGGAAACCTCCTCGG |
| | | ATTCCATTGCCCAGCTATCTGTCACTTCATCAAAAGGACAGTAGAAA |
| | | AGGAAGGTGGCACCTACAAATGCCATCATTGCGATAAAGGAAAGG |
| | | CTATCGTTCAAGATGCCTCTGCCGACAGTGGTCCCAAAGATGGACC |
| | | CCCACCCACGAGGAGCATCGTGGAAAAAGAAGACGTTCCAACCAC |
| | | GTCTTCAAAGCAAGTGGATTGATGTGATATCTCCACTGACGTAAGG |
| | | GATGACGCACAATCCCACTATCCTTCGCAAGACCCTTCCTCTATATA |
| | | AGGAAGTTCATTTCATTTGGAGAGGA |
| 5 | 2xGal4sp10FMV | CCTAGTAGCTTCCGGAAGACTCTCCTCCGTAATGCTTCGCGGAAGA |
| | | CTCTCCTCCGaagcttAGGATTTAGCAGCATTCCAGATTGGGTTCAAT |
| | | CAACAAGGTACGAGCCATATCACTTTATTCAAATTGGTATCGCCAAA |
| | | ACCAAGAAGGAACTCCCATCCTCAAAGGTTTGTAAGGAAGAATTCT |
| | | CAGTCCAAAGCCTCAACAAGGTCAGGGTACAGAGTCTCCAAACCAT |
| | | TAGCCAAAAGCTACAGGAGATCAATGAAGAATCTTCAATCAAAGTAA |
| | | ACTACTGTTCCAGCACATGCATCATGGTCAGTAAGTTTCAGAAAAA |
| | | GACATCCACCGAAGACTTAAAGTTAGTGGGCATCTTTGAAAGTAAT |
| | | CTTGTCAACATCGAGCAGCTGGCTTGTGGGGACCAGACAAAAAAG |
| | | GAATGGTGCAGAATTGTTAGGCGCACCTACCAAAAGCATCTTTGCC |
| | | TTTATTGCAAAGATAAAGCAGATTCCTCTAGTACAAGTGGGGAACAA |
| | | AATAACGTGGAAAAGAGCTGTCCTGACAGCCCACTCACTAATGCGT |
| | | ATGACGAACGCAGTGACGACCACAAAAGAATTCCCTCTATATAAGA |
| | | AGGCATTCATTCCCATTTGAAGGATCATCAGATACTCAAC |
| 6 | 2xGal4sp10NOS | CGGAAGACTCTCCTCCGTAATGCTTCGCGGAAGACTCTCCTCCGAA |
| | | GCTTAGGCGGGAAACGACAATCTGATCATGAGCGGAGAATTAAGG |
| | | GAGTCACGTTATGACCCCCGCCGATGACGCGGGACAAGCCGTTTT |
| | | ACGTTTGGAACTGACAGAACCGCAACGATTGAAGGAGCCACTCAG |
| | | CCGCGGGTTTCTGGAGTTTAATGAGCTAAGCACATACGTCAGAAAC |
| | | CATTATTGCGCGTTCAAAAGTCGCCTAAGGTCACTATCAGCTAGCA |
| | | AATATTTCTTGTCAAAAATGCTCCACTGACGTTCCATAAATTCCCCT |
| | | CGGTATCCAATTAGAGTCTCATATTCACTCTCAATCCAAATAATCTG |
| | | CACCG |
| 7 | 2xLexA35S | ACTGTACATATAACCACTGGTTTTATATACAGCAGTaagcttTGAGACT |
| | | TTTCAACAAAGGGTAATATCGGGAAACCTCCTCGGATTCCATTGCC |
| | | CAGCTATCTGTCACTTCATCAAAAGGACAGTAGAAAAGGAAGGTGG |
| | | CACCTACAAATGCCATCATTGCGATAAAGGAAAGGCTATCGTTCAA |
| | | GATGCCTCTGCCGACAGTGGTCCCAAAGATGGACCCCCACCCACG |
| | | AGGAGCATCGTGGAAAAAGAAGACGTTCCAACCACGTCTTCAAAGC |
| | | AAGTGGATTGATGTGATATCTCCACTGACGTAAGGGATGACGCACA |
| | | ATCCCACTATCCTTCGCAAGACCCTTCCTCTATATAAGGAAGTTCAT |
| | | TTCATTTGGAGAGGA |
| 8 | 2xLexAFMV | ACTGTACATATAACCACTGGTTTTATATACAGCAGTaagcttAGGATTT |
| | | AGCAGCATTCCAGATTGGGTTCAATCAACAAGGTACGAGCCATATC |
| | | ACTTTATTCAAATTGGTATCGCCAAAACCAAGAAGGAACTCCCATCC |
| | | TCAAAGGTTTGTAAGGAAGAATTCTCAGTCCAAAGCCTCAACAAGG |
| | | TCAGGGTACAGAGTCTCCAAACCATTAGCCAAAAGCTACAGGAGAT |
| | | CAATGAAGAATCTTCAATCAAAGTAAACTACTGTTCCAGCACATGCA |
| | | TCATGGTCAGTAAGTTTCAGAAAAAGACATCCACCGAAGACTTAAA |
| | | GTTAGTGGGCATCTTTGAAAGTAATCTTGTCAACATCGAGCAGCTG |
| | | GCTTGTGGGGACCAGACAAAAAAGGAATGGTGCAGAATTGTTAGG |
| | | CGCACCTACCAAAAGCATCTTTGCCTTTATTGCAAAGATAAAGCAGA |
| | | TTCCTCTAGTACAAGTGGGGAACAAAATAACGTGGAAAAGAGCTGT |
| | | CCTGACAGCCCACTCACTAATGCGTATGACGAACGCAGTGACGAC |
| | | CACAAAAGAATTCCCTCTATATAAGAAGGCATTCATTCCCATTTGAA |
| | | GGATCATCAGATACTCAACG |
| 9 | 2xLexANOS | ACTGTACATATAACCACTGGTTTTATATACAGCAGTaagcttAGGCGG |
| | | GAAACGACAATCTGATCATGAGCGGAGAATTAAGGGAGTCACGTTA |
| | | TGACCCCCGCCGATGACGCGGGACAAGCCGTTTTACGTTTGGAAC |
| | | TGACAGAACCGCAACGATTGAAGGAGCCACTCAGCCGCGGGTTTC |
| | | TGGAGTTTAATGAGCTAAGCACATACGTCAGAAACCATTATTGCGC |
| | | GTTCAAAAGTCGCCTAAGGTCACTATCAGCTAGCAAATATTTCTTGT |
| | | CAAAAATGCTCCACTGACGTTCCATAAATTCCCCTCGGTATCCAATT |
| | | AGAGTCTCATATTCACTCTCAATCCAAATAATCTGCACCG |
| 10 | 35S2xGal4 | TGAGACTTTTCAACAAAGGGTAATATCGGGAAACCTCCTCGGATTC |
| | | CATTGCCCAGCTATCTGTCACTTCATCAAAAGGACAGTAGAAAAGG |
| | | AAGGTGGCACCTACAAATGCCATCATTGCGATAAAGGAAAGGCTAT |
| | | CGTTCAAGATGCCTCTGCCGACAGTGGTCCCAAAGATGGACCCCC |
| | | ACCCACGAGGAGCATCGTGGAAAAAGAAGACGTTCCAACCACGTC |
| | | TTCAAAGCAAGTGGATTGATGTGATATCTCCACTGACGTAAGGGAT |
| | | GACGCACAATCCCACTATCCTTCGCAAGACCCTTCCTCTATATAAG |
| | | GAAGTTCATTTCATTTGGAGAGGAacgcgtCGGAAGACTCTCCTCCGA |
| | | GCGGAAGACTCTCCTCCG |
| 11 | 35S2xGal4sp10 | TGAGACTTTTCAACAAAGGGTAATATCGGGAAACCTCCTCGGATTC |
| | | CATTGCCCAGCTATCTGTCACTTCATCAAAAGGACAGTAGAAAAGG |
| | | AAGGTGGCACCTACAAATGCCATCATTGCGATAAAGGAAAGGCTAT |
| | | CGTTCAAGATGCCTCTGCCGACAGTGGTCCCAAAGATGGACCCCC |
| | | ACCCACGAGGAGCATCGTGGAAAAAGAAGACGTTCCAACCACGTC |
| | | TTCAAAGCAAGTGGATTGATGTGATATCTCCACTGACGTAAGGGAT |
| | | GACGCACAATCCCACTATCCTTCGCAAGACCCTTCCTCTATATAAG |
| | | GAAGTTCATTTCATTTGGAGAGGAacgcgtCCTAGTAGCTTCCGGAAG |
| | | ACTCTCCTCCGTAATGCTTCGCGGAAGACTCTCCTCCG |
| 12 | 35S2xLexA | TGAGACTTTTCAACAAAGGGTAATATCGGGAAACCTCCTCGGATTC |
| | | CATTGCCCAGCTATCTGTCACTTCATCAAAAGGACAGTAGAAAAGG |
| | | AAGGTGGCACCTACAAATGCCATCATTGCGATAAAGGAAAGGCTAT |
| | | CGTTCAAGATGCCTCTGCCGACAGTGGTCCCAAAGATGGACCCCC |
| | | ACCCACGAGGAGCATCGTGGAAAAAGAAGACGTTCCAACCACGTC |
| | | TTCAAAGCAAGTGGATTGATGTGATATCTCCACTGACGTAAGGGAT |
| | | GACGCACAATCCCACTATCCTTCGCAAGACCCTTCCTCTATATAAG |
| | | GAAGTTCATTTCATTTGGAGAGGAacgcgtACTGTACATATAACCACT |
| | | GGTTTTATATACAGCAGT |
| 13 | 35S4xLexATATA | TGAGACTTTTCAACAAAGGGTAATATCGGGAAACCTCCTCGGATTC |
| | | CATTGCCCAGCTATCTGTCACTTCATCAAAAGGACAGTAGAAAAGG |
| | | AAGGTGGCACCTACAAATGCCATCATTGCGATAAAGGAAAGGCTAT |
| | | CGTTCAAGATGCCTCTGCCGACAGTGGTCCCAAAGATGGACCCCC |
| | | ACCCACGAGGAGCATCGTGGAAAAAGAAGACGTTCCAACCACGTC |
| | | TTCAAAGCAAGTGGATTGATGTGATATCTCCACTGACGTAAGGGAT |
| | | GACGCACAATCCCACTATCCTTCGCAAGACCCTTCCTCACTGTACA |
| | | TATAACCACTGGTTTTATATACAGCAGTACTGTACATATAACCACTG |
| | | GTTTTATATACAGCAGTTATATAAGGAAGTTCATTTCATTTGGAGAG |
| | | GA |
| 14 | 35S5xGal4TATA | TGAGACTTTTCAACAAAGGGTAATATCGGGAAACCTCCTCGGATTC |
| | | CATTGCCCAGCTATCTGTCACTTCATCAAAAGGACAGTAGAAAAGG |
| | | AAGGTGGCACCTACAAATGCCATCATTGCGATAAAGGAAAGGCTAT |
| | | CGTTCAAGATGCCTCTGCCGACAGTGGTCCCAAAGATGGACCCCC |
| | | ACCCACGAGGAGCATCGTGGAAAAAGAAGACGTTCCAACCACGTC |
| | | TTCAAAGCAAGTGGATTGATGTGATATCTCCACTGACGTAAGGGAT |
| | | GACGCACAATCCCACTATCCTTCGCAAGACCCTTCCTCCGGAAGAC |
| | | TCTCCTCCGAGCGGAAGACTCTCCTCCGAGCGGAAGACTCTCCTC |
| | | CAGGCGGAAGACTCTCCTCCGAGCGGAAGACTCTCCTCCGTATAT |
| | | AAGGAAGTTCATTTCATTTGGAGAGGA |
| 15 | 8xLexAFMV | ACTGTACATATAACCACTGGTTTTATATACAGCAGTACTGTACATAT |
| | | AACCACTGGTTTTATATACAGCAGTCGACGTACTGTACATATAACCA |
| | | CTGGTTTTATATACAGCAGTACTGTACATATAACCACTGGTTTTATAT |
| | | ACAGCAGTCaagcttAGGATTTAGCAGCATTCCAGATTGGGTTCAATC |
| | | AACAAGGTACGAGCCATATCACTTTATTCAAATTGGTATCGCCAAAA |
| | | CCAAGAAGGAACTCCCATCCTCAAAGGTTTGTAAGGAAGAATTCTC |
| | | AGTCCAAAGCCTCAACAAGGTCAGGGTACAGAGTCTCCAAACCATT |
| | | AGCCAAAAGCTACAGGAGATCAATGAAGAATCTTCAATCAAAGTAA |
| | | ACTACTGTTCCAGCACATGCATCATGGTCAGTAAGTTTCAGAAAAA |
| | | GACATCCACCGAAGACTTAAAGTTAGTGGGCATCTTTGAAAGTAAT |
| | | CTTGTCAACATCGAGCAGCTGGCTTGTGGGGACCAGACAAAAAAG |
| | | GAATGGTGCAGAATTGTTAGGCGCACCTACCAAAAGCATCTTTGCC |
| | | TTTATTGCAAAGATAAAGCAGATTCCTCTAGTACAAGTGGGGAACAA |
| | | AATAACGTGGAAAAGAGCTGTCCTGACAGCCCACTCACTAATGCGT |
| | | ATGACGAACGCAGTGACGACCACAAAAGAATTCCCTCTATATAAGA |
| | | AGGCATTCATTCCCATTTGAAGGATCATCAGATACTCAACG |
| 16 | 8xLexA35S | ACTGTACATATAACCACTGGTTTTATATACAGCAGTACTGTACATAT |
| | | AACCACTGGTTTTATATACAGCAGTCGACGTACTGTACATATAACCA |
| | | CTGGTTTTATATACAGCAGTACTGTACATATAACCACTGGTTTTATAT |
| | | ACAGCAGTCaagcttTGAGACTTTTCAACAAAGGGTAATATCGGGAAA |
| | | CCTCCTCGGATTCCATTGCCCAGCTATCTGTCACTTCATCAAAAGG |
| | | ACAGTAGAAAAGGAAGGTGGCACCTACAAATGCCATCATTGCGATA |
| | | AAGGAAAGGCTATCGTTCAAGATGCCTCTGCCGACAGTGGTCCCAA |
| | | AGATGGACCCCCACCCACGAGGAGCATCGTGGAAAAAGAAGACGT |
| | | TCCAACCACGTCTTCAAAGCAAGTGGATTGATGTGATATCTCCACT |
| | | GACGTAAGGGATGACGCACAATCCCACTATCCTTCGCAAGACCCTT |
| | | CCTCTATATAAGGAAGTTCATTTCATTTGGAGAGGA |
| 17 | 8xLexANOS | ACTGTACATATAACCACTGGTTTTATATACAGCAGTACTGTACATAT |
| | | AACCACTGGTTTTATATACAGCAGTCGACGTACTGTACATATAACCA |
| | | CTGGTTTTATATACAGCAGTACTGTACATATAACCACTGGTTTTATAT |
| | | ACAGCAGTCaagcttAGGCGGGAAACGACAATCTGATCATGAGCGGA |
| | | GAATTAAGGGAGTCACGTTATGACCCCCGCCGATGACGCGGGACA |
| | | AGCCGTTTTACGTTTGGAACTGACAGAACCGCAACGATTGAAGGAG |
| | | CCACTCAGCCGCGGGTTTCTGGAGTTTAATGAGCTAAGCACATACG |
| | | TCAGAAACCATTATTGCGCGTTCAAAAGTCGCCTAAGGTCACTATC |
| | | AGCTAGCAAATATTTCTTGTCAAAAATGCTCCACTGACGTTCCATAA |
| | | ATTCCCCTCGGTATCCAATTAGAGTCTCATATTCACTCTCAATCCAA |
| | | ATAATCTGCACCG |
| 18 | 8xLexAsp1035S | ACTGTACATATAACCACTGGTTTTATATACAGCAGTTAATGCTTCGA |
| | | CTGTACATATAACCACTGGTTTTATATACAGCAGTTAATGCTTCGAC |
| | | TGTACATATAACCACTGGTTTTATATACAGCAGTTAATGCTTCGACT |
| | | GTACATATAACCACTGGTTTTATATACAGCAGTATCGAGAGACCTGA |
| | | GACTTTTCAACAAAGGGTAATATCGGGAAACCTCCTCGGATTCCAT |
| | | TGCCCAGCTATCTGTCACTTCATCAAAAGGACAGTAGAAAAGGAAG |
| | | GTGGCACCTACAAATGCCATCATTGCGATAAAGGAAAGGCTATCGT |
| | | TCAAGATGCCTCTGCCGACAGTGGTCCCAAAGATGGACCCCCACC |
| | | CACGAGGAGCATCGTGGAAAAAGAAGACGTTCCAACCACGTCTTCA |
| | | AAGCAAGTGGATTGATGTGATATCTCCACTGACGTAAGGGATGACG |
| | | CACAATCCCACTATCCTTCGCAAGACCCTTCCTCTATATAAGGAAGT |
| | | TCATTTCATTTGGAGAGG |
| 19 | 8xLexAsp10FMV | ACTGTACATATAACCACTGGTTTTATATACAGCAGTTAATGCTTCGA |
| | | CTGTACATATAACCACTGGTTTTATATACAGCAGTTAATGCTTCGAC |
| | | TGTACATATAACCACTGGTTTTATATACAGCAGTTAATGCTTCGACT |
| | | GTACATATAACCACTGGTTTTATATACAGCAGTaagcttAGGATTTAGC |
| | | AGCATTCCAGATTGGGTTCAATCAACAAGGTACGAGCCATATCACT |
| | | TTATTCAAATTGGTATCGCCAAAACCAAGAAGGAACTCCCATCCTCA |
| | | AAGGTTTGTAAGGAAGAATTCTCAGTCCAAAGCCTCAACAAGGTCA |
| | | GGGTACAGAGTCTCCAAACCATTAGCCAAAAGCTACAGGAGATCAA |
| | | TGAAGAATCTTCAATCAAAGTAAACTACTGTTCCAGCACATGCATCA |
| | | TGGTCAGTAAGTTTCAGAAAAAGACATCCACCGAAGACTTAAAGTTA |
| | | GTGGGCATCTTTGAAAGTAATCTTGTCAACATCGAGCAGCTGGCTT |
| | | GTGGGGACCAGACAAAAAAGGAATGGTGCAGAATTGTTAGGCGCA |
| | | CCTACCAAAAGCATCTTTGCCTTTATTGCAAAGATAAAGCAGATTCC |
| | | TCTAGTACAAGTGGGGAACAAAATAACGTGGAAAAGAGCTGTCCTG |
| | | ACAGCCCACTCACTAATGCGTATGACGAACGCAGTGACGACCACAA |
| | | AAGAATTCCCTCTATATAAGAAGGCATTCATTCCCATTTGAAGGATC |
| | | ATCAGATACTCAAC |
| 20 | 8xLexAsp10NOS | ACTGTACATATAACCACTGGTTTTATATACAGCAGTTAATGCTTCGA |
| | | CTGTACATATAACCACTGGTTTTATATACAGCAGTTAATGCTTCGAC |
| | | TGTACATATAACCACTGGTTTTATATACAGCAGTTAATGCTTCGACT |
| | | GTACATATAACCACTGGTTTTATATACAGCAGTATCGAGAGACCAG |
| | | GCGGGAAACGACAATCTGATCATGAGCGGAGAATTAAGGGAGTCA |
| | | CGTTATGACCCCCGCCGATGACGCGGGACAAGCCGTTTTACGTTT |
| | | GGAACTGACAGAACCGCAACGATTGAAGGAGCCACTCAGCCGCGG |
| | | GTTTCTGGAGTTTAATGAGCTAAGCACATACGTCAGAAACCATTATT |
| | | GCGCGTTCAAAAGTCGCCTAAGGTCACTATCAGCTAGCAAATATTT |
| | | CTTGTCAAAAATGCTCCACTGACGTTCCATAAATTCCCCTCGGTATC |
| | | CAATTAGAGTCTCATATTCACTCTCAATCCAAATAATCTGCACCG |
| 21 | FMV2xGal4 | AGGATTTAGCAGCATTCCAGATTGGGTTCAATCAACAAGGTACGAG |
| | | CCATATCACTTTATTCAAATTGGTATCGCCAAAACCAAGAAGGAACT |
| | | CCCATCCTCAAAGGTTTGTAAGGAAGAATTCTCAGTCCAAAGCCTC |
| | | AACAAGGTCAGGGTACAGAGTCTCCAAACCATTAGCCAAAAGCTAC |
| | | AGGAGATCAATGAAGAATCTTCAATCAAAGTAAACTACTGTTCCAGC |
| | | ACATGCATCATGGTCAGTAAGTTTCAGAAAAAGACATCCACCGAAG |
| | | ACTTAAAGTTAGTGGGCATCTTTGAAAGTAATCTTGTCAACATCGAG |
| | | CAGCTGGCTTGTGGGGACCAGACAAAAAAGGAATGGTGCAGAATT |
| | | GTTAGGCGCACCTACCAAAAGCATCTTTGCCTTTATTGCAAAGATAA |
| | | AGCAGATTCCTCTAGTACAAGTGGGGAACAAAATAACGTGGAAAAG |
| | | AGCTGTCCTGACAGCCCACTCACTAATGCGTATGACGAACGCAGTG |
| | | ACGACCACAAAAGAATTCCCTCTATATAAGAAGGCATTCATTCCCAT |
| | | TTGAAGGATCATCAGATACTCAACGacgcgtCGGAAGACTCTCCTCCG |
| | | AGCGGAAGACTCTCCTCCG |
| 22 | FMV2xGal4sp10 | AGGATTTAGCAGCATTCCAGATTGGGTTCAATCAACAAGGTACGAG |
| | | CCATATCACTTTATTCAAATTGGTATCGCCAAAACCAAGAAGGAACT |
| | | CCCATCCTCAAAGGTTTGTAAGGAAGAATTCTCAGTCCAAAGCCTC |
| | | AACAAGGTCAGGGTACAGAGTCTCCAAACCATTAGCCAAAAGCTAC |
| | | AGGAGATCAATGAAGAATCTTCAATCAAAGTAAACTACTGTTCCAGC |
| | | ACATGCATCATGGTCAGTAAGTTTCAGAAAAAGACATCCACCGAAG |
| | | ACTTAAAGTTAGTGGGCATCTTTGAAAGTAATCTTGTCAACATCGAG |
| | | CAGCTGGCTTGTGGGGACCAGACAAAAAAGGAATGGTGCAGAATT |
| | | GTTAGGCGCACCTACCAAAAGCATCTTTGCCTTTATTGCAAAGATAA |
| | | AGCAGATTCCTCTAGTACAAGTGGGGAACAAAATAACGTGGAAAAG |
| | | AGCTGTCCTGACAGCCCACTCACTAATGCGTATGACGAACGCAGTG |
| | | ACGACCACAAAAGAATTCCCTCTATATAAGAAGGCATTCATTCCCAT |
| | | TTGAAGGATCATCAGATACTCAACGacgcgtCCTAGTAGCTTCCGGAA |
| | | GACTCTCCTCCGTAATGCTTCGCGGAAGACTCTCCTCCG |
| 23 | FMV2xLexA | AGGATTTAGCAGCATTCCAGATTGGGTTCAATCAACAAGGTACGAG |
| | | CCATATCACTTTATTCAAATTGGTATCGCCAAAACCAAGAAGGAACT |
| | | CCCATCCTCAAAGGTTTGTAAGGAAGAATTCTCAGTCCAAAGCCTC |
| | | AACAAGGTCAGGGTACAGAGTCTCCAAACCATTAGCCAAAAGCTAC |
| | | AGGAGATCAATGAAGAATCTTCAATCAAAGTAAACTACTGTTCCAGC |
| | | ACATGCATCATGGTCAGTAAGTTTCAGAAAAAGACATCCACCGAAG |
| | | ACTTAAAGTTAGTGGGCATCTTTGAAAGTAATCTTGTCAACATCGAG |
| | | CAGCTGGCTTGTGGGGACCAGACAAAAAAGGAATGGTGCAGAATT |
| | | GTTAGGCGCACCTACCAAAAGCATCTTTGCCTTTATTGCAAAGATAA |
| | | AGCAGATTCCTCTAGTACAAGTGGGGAACAAAATAACGTGGAAAAG |
| | | AGCTGTCCTGACAGCCCACTCACTAATGCGTATGACGAACGCAGTG |
| | | ACGACCACAAAAGAATTCCCTCTATATAAGAAGGCATTCATTCCCAT |
| | | TTGAAGGATCATCAGATACTCAACGacgcgtACTGTACATATAACCAC |
| | | TGGTTTTATATACAGCAGT |
| 24 | NOS2xGal4 | AGGCGGGAAACGACAATCTGATCATGAGCGGAGAATTAAGGGAGT |
| | | CACGTTATGACCCCCGCCGATGACGCGGGACAAGCCGTTTTACGT |
| | | TTGGAACTGACAGAACCGCAACGATTGAAGGAGCCACTCAGCCGC |
| | | GGGTTTCTGGAGTTTAATGAGCTAAGCACATACGTCAGAAACCATT |
| | | ATTGCGCGTTCAAAAGTCGCCTAAGGTCACTATCAGCTAGCAAATA |
| | | TTTCTTGTCAAAAATGCTCCACTGACGTTCCATAAATTCCCCTCGGT |
| | | ATCCAATTAGAGTCTCATATTCACTCTCAATCCAAATAATCTGCACC |
| | | GacgcgtCGGAAGACTCTCCTCCGAGCGGAAGACTCTCCTCCG |
| 25 | NOS2xGal4sp10 | AGGCGGGAAACGACAATCTGATCATGAGCGGAGAATTAAGGGAGT |
| | | CACGTTATGACCCCCGCCGATGACGCGGGACAAGCCGTTTTACGT |
| | | TTGGAACTGACAGAACCGCAACGATTGAAGGAGCCACTCAGCCGC |
| | | GGGTTTCTGGAGTTTAATGAGCTAAGCACATACGTCAGAAACCATT |
| | | ATTGCGCGTTCAAAAGTCGCCTAAGGTCACTATCAGCTAGCAAATA |
| | | TTTCTTGTCAAAAATGCTCCACTGACGTTCCATAAATTCCCCTCGGT |
| | | ATCCAATTAGAGTCTCATATTCACTCTCAATCCAAATAATCTGCACC |
| | | GacgcgtCCTAGTAGCTTCCGGAAGACTCTCCTCCGTAATGCTTCGC |
| | | GGAAGACTCTCCTCCG |
| 26 | NOS2xLexA | AGGCGGGAAACGACAATCTGATCATGAGCGGAGAATTAAGGGAGT |
| | | CACGTTATGACCCCCGCCGATGACGCGGGACAAGCCGTTTTACGT |
| | | TTGGAACTGACAGAACCGCAACGATTGAAGGAGCCACTCAGCCGC |
| | | GGGTTTCTGGAGTTTAATGAGCTAAGCACATACGTCAGAAACCATT |
| | | ATTGCGCGTTCAAAAGTCGCCTAAGGTCACTATCAGCTAGCAAATA |
| | | TTTCTTGTCAAAAATGCTCCACTGACGTTCCATAAATTCCCCTCGGT |
| | | ATCCAATTAGAGTCTCATATTCACTCTCAATCCAAATAATCTGCACC |
| | | GacgcgtACTGTACATATAACCACTGGTTTTATATACAGCAGT |
| 27 | Gal4BRD | ATGAAGTTGTTAAGTTCTATTGAACAGGCTTGTGATATTTGTAGATT |
| | | GAAGAAGCTCAAGTGTAGTAAAGAGAAACCTAAGTGCGCTAAGTGT |
| | | CTTAAAAATAACTGGGAGTGCAGATATTCTCCTAAGACTAAGAGATC |
| | | ACCTCTTACTAGGGCTCATCTCACAGAGGTGGAGTCTAGGCTTGAG |
| | | AGATTGGAGCAGTTGTTCCTTTTGATTTTTCCAAGAGAAGATCTTGA |
| | | TATGATTCTTAAGATGGATTCTCTTCAAGATATTAAGGCTCTTCTTAC |
| | | TGGTTTATTCGTTCAAGATAATGTGAATAAGGATGCAGTGACTGATA |
| | | GACTTGCATCAGTTGAAACTGATATGCCTCTTACATTAAGACAACAC |
| | | AGGATTTCTGCTACTTCAAGTTCTGAAGAAAGTTCTAATAAGGGTCA |
| | | AAGGCAATTAACCGTTTCTTTAAGACTGTTCGGAGTGAACATGTGA |
| 28 | Gal4EAR | ATGAAGTTGTTAAGTTCTATTGAACAGGCTTGTGATATTTGTAGATT |
| | | GAAGAAGCTCAAGTGTAGTAAAGAGAAACCTAAGTGCGCTAAGTGT |
| | | CTTAAAAATAACTGGGAGTGCAGATATTCTCCTAAGACTAAGAGATC |
| | | ACCTCTTACTAGGGCTCATCTCACAGAGGTGGAGTCTAGGCTTGAG |
| | | AGATTGGAGCAGTTGTTCCTTTTGATTTTTCCAAGAGAAGATCTTGA |
| | | TATGATTCTTAAGATGGATTCTCTTCAAGATATTAAGGCTCTTCTTAC |
| | | TGGTTTATTCGTTCAAGATAATGTGAATAAGGATGCAGTGACTGATA |
| | | GACTTGCATCAGTTGAAACTGATATGCCTCTTACATTAAGACAACAC |
| | | AGGATTTCTGCTACTTCAAGTTCTGAAGAAAGTTCTAATAAGGGTCA |
| | | AAGGCAATTAACCGTTTCTCTTGATTTGGACCTCGAGCTTCGTTTGG |
| | | GATTCGCTTGA |
| 29 | Gal4OFP1 | ATGAAGTTGTTAAGTTCTATTGAACAGGCTTGTGATATTTGTAGATT |
| | | GAAGAAGCTCAAGTGTAGTAAAGAGAAACCTAAGTGCGCTAAGTGT |
| | | CTTAAAAATAACTGGGAGTGCAGATATTCTCCTAAGACTAAGAGATC |
| | | ACCTCTTACTAGGGCTCATCTCACAGAGGTGGAGTCTAGGCTTGAG |
| | | AGATTGGAGCAGTTGTTCCTTTTGATTTTTCCAAGAGAAGATCTTGA |
| | | TATGATTCTTAAGATGGATTCTCTTCAAGATATTAAGGCTCTTCTTAC |
| | | TGGTTTATTCGTTCAAGATAATGTGAATAAGGATGCAGTGACTGATA |
| | | GACTTGCATCAGTTGAAACTGATATGCCTCTTACATTAAGACAACAC |
| | | AGGATTTCTGCTACTTCAAGTTCTGAAGAAAGTTCTAATAAGGGTCA |
| | | AAGGCAATTAACCGTTTCTAGCAGGGCGGTGGTGAAGGCGTCGGT |
| | | GGATCCGAAGAGAGACTTTAAGGAGTCGATGGAGGAGATGATCGC |
| | | TGAGAACAAGATAAGAGCGACAAAGGATCTAGAGGAGCTTTTGGCT |
| | | TGCTATCTCTGTCTCAATTCAGACGAATATCACGCTATTATCATCAA |
| | | TGTCTTCAAGCAAATCTGGCTTGATCTTAATCTTCCA |
| 30 | Gal4OFPX | ATGAAGTTGTTAAGTTCTATTGAACAGGCTTGTGATATTTGTAGATT |
| | | GAAGAAGCTCAAGTGTAGTAAAGAGAAACCTAAGTGCGCTAAGTGT |
| | | CTTAAAAATAACTGGGAGTGCAGATATTCTCCTAAGACTAAGAGATC |
| | | ACCTCTTACTAGGGCTCATCTCACAGAGGTGGAGTCTAGGCTTGAG |
| | | AGATTGGAGCAGTTGTTCCTTTTGATTTTTCCAAGAGAAGATCTTGA |
| | | TATGATTCTTAAGATGGATTCTCTTCAAGATATTAAGGCTCTTCTTAC |
| | | TGGTTTATTCGTTCAAGATAATGTGAATAAGGATGCAGTGACTGATA |
| | | GACTTGCATCAGTTGAAACTGATATGCCTCTTACATTAAGACAACAC |
| | | AGGATTTCTGCTACTTCAAGTTCTGAAGAAAGTTCTAATAAGGGTCA |
| | | AAGGCAATTAACCGTTTCTAGCTTGGCGGTGGTGAAGAAGTCGGTG |
| | | GATCCAAACAAAGATTTCAGGGAATCAATGGTGGAGATGATAGCAG |
| | | AGAACAAGATAAGAGCATCAAATGACCTAGAAGAGCTTCTTGCTTG |
| | | CTACCTTTCGTTAAATCCAAAGGAATATCACGATCTTATTATCAAAG |
| | | TGTTCGAACAAATCTGGCTTGAACTGATAAACCCA |
| 31 | LexABRD | ATGAAAGCTCTTACTGCTAGACAACAAGAAGTTTTTGATTTGATTAG |
| | | GGATCATATTTCTCAGACAGGTATGCCTCCTACTAGAGCTGAGATC |
| | | GCTCAGAGACTCGGTTTCAGATCTCCTAACGCTGCTGAAGAGCATC |
| | | TTAAGGCTCTTGCTAGAAAGGGAGTTATTGAGATTGTGAGTGGAGC |
| | | ATCAAGAGGTATTAGGTTGCTTCAAGAGGAAGAAGAGGGACTTCCT |
| | | CTTGTTGGTAGAGTTGCAGCTGGTGAGCCTTTAAGACTGTTCGGAG |
| | | TGAACATGTGA |
| 32 | LexAEAR | ATGAAAGCTCTTACTGCTAGACAACAAGAAGTTTTTGATTTGATTAG |
| | | GGATCATATTTCTCAGACAGGTATGCCTCCTACTAGAGCTGAGATC |
| | | GCTCAGAGACTCGGTTTCAGATCTCCTAACGCTGCTGAAGAGCATC |
| | | TTAAGGCTCTTGCTAGAAAGGGAGTTATTGAGATTGTGAGTGGAGC |
| | | ATCAAGAGGTATTAGGTTGCTTCAAGAGGAAGAAGAGGGACTTCCT |
| | | CTTGTTGGTAGAGTTGCAGCTGGTGAGCCTCTTGATTTGGACCTCG |
| | | AGCTTCGTTTGGGATTCGCTTGA |
| 33 | LexAOFP1 | ATGAAAGCTCTTACTGCTAGACAACAAGAAGTTTTTGATTTGATTAG |
| | | GGATCATATTTCTCAGACAGGTATGCCTCCTACTAGAGCTGAGATC |
| | | GCTCAGAGACTCGGTTTCAGATCTCCTAACGCTGCTGAAGAGCATC |
| | | TTAAGGCTCTTGCTAGAAAGGGAGTTATTGAGATTGTGAGTGGAGC |
| | | ATCAAGAGGTATTAGGTTGCTTCAAGAGGAAGAAGAGGGACTTCCT |
| | | CTTGTTGGTAGAGTTGCAGCTGGTGAGCCTAGCAGGGCGGTGGTG |
| | | AAGGCGTCGGTGGATCCGAAGAGAGACTTTAAGGAGTCGATGGAG |
| | | GAGATGATCGCTGAGAACAAGATAAGAGCGACAAAGGATCTAGAG |
| | | GAGCTTTTGGCTTGCTATCTCTGTCTCAATTCAGACGAATATCACGC |
| | | TATTATCATCAATGTCTTCAAGCAAATCTGGCTTGATCTTAATCTTCC |
| | | A |
| 34 | LexAOFPX | ATGAAAGCTCTTACTGCTAGACAACAAGAAGTTTTTGATTTGATTAG |
| | | GGATCATATTTCTCAGACAGGTATGCCTCCTACTAGAGCTGAGATC |
| | | GCTCAGAGACTCGGTTTCAGATCTCCTAACGCTGCTGAAGAGCATC |
| | | TTAAGGCTCTTGCTAGAAAGGGAGTTATTGAGATTGTGAGTGGAGC |
| | | ATCAAGAGGTATTAGGTTGCTTCAAGAGGAAGAAGAGGGACTTCCT |
| | | CTTGTTGGTAGAGTTGCAGCTGGTGAGCCTAGCTTGGCGGTGGTG |
| | | AAGAAGTCGGTGGATCCAAACAAAGATTTCAGGGAATCAATGGTGG |
| | | AGATGATAGCAGAGAACAAGATAAGAGCATCAAATGACCTAGAAGA |
| | | GCTTCTTGCTTGCTACCTTTCGTTAAATCCAAAGGAATATCACGATC |
| | | TTATTATCAAAGTGTTCGAACAAATCTGGCTTGAACTGATAAACCCA |
| 35 | AtOFPx | AGCTTGGCGGTGGTGAAGAAGTCGGTGGATCCAAACAAAGATTTCA |
| | | GGGAATCAATGGTGGAGATGATAGCAGAGAACAAGATAAGAGCAT |
| | | CAAATGACCTAGAAGAGCTTCTTGCTTGCTACCTTTCGTTAAATCCA |
| | | AAGGAATATCACGATCTTATTATCAAAGTGTTCGAACAAATCTGGCT |
| | | TGAACTGATAAACCCA |

### EXAMPLES

The following examples illustrate various iterations of the invention.

### Background

Synthetic Biology promises to bring new understanding of living organisms while allowing the design of predictable biological function. Yet to date all quantitatively defined genetic circuits have been produced in unicellular organisms (bacteria, yeast) or mammalian cells in culture¹⁻⁴. This raises a question as to whether synthetic genetic circuits with predictable function can be produced in multicellular organisms. In multicellular organisms, sexual reproduction proceeds through meiosis, and in plants this is accompanied by further development into gametophyte and sporophyte stages, potentially affecting the stability and function of synthetic genetic elements in the genome. Even so, genetic circuits with highly predictable function in plants could have profound applications towards sustainable life on earth. For example, such circuits could be used to control biofuel production so that optimal biomass is produced prior to the trait induction. Moreover, it is unlikely that natural production of biomaterials is optimum. For example, cotton fibers are only produced from ovule epidermal cells rather than the considerably more abundant epidermal cells of leaves.

The ability to design and produce predictable genetic circuits in plants requires a deep understanding of plant biology and rigorous quantitative data. The latter requirement imposes a significant challenge that lead some to believe that quantitative predictable function of synthetic genetic circuits in plants is unattainable. The concerns are myriad. Plants develop continuously, move regulatory molecules between cells and tissues, and control much of their differentiation by positional information with input from their local environment⁵. Plant epigenetics, known to affect many processes, is not fully understood⁶. Hence, a predictable genetic circuit should function independent of inputs from endogenous genetic and epigenetic regulators, positional information, and the environment.

The standardized parts-based approach used in synthetic biology requires accurate quantification of the dynamic behavior of each part in vivo in order to rationally and predictably design higher-level complex circuits. Therefore, the first step towards producing predictable function in higher-level circuits is quantification of the input-output characteristics of genetic parts.

Quantitative analysis of a large number of stably integrated genetic parts (e.g., promoters, terminators, UTRs) in plants would require decades of efforts, even for fast growing species such as Arabidopsis. Current methods for rapid transient gene expression in higher plants, such as particle bombardment, Agrobacterium infiltration, VIGS (virus-induced gene silencing)-based systems, and protoplasts⁷⁻¹⁰ could be used for parts characterization. However, particle bombardment and VIGS do not easily allow high throughput analysis. Agrobacterium infiltration methods, including AGROBEST¹¹, could be scaled up to test a large number of parts, but quantification of function is difficult. Transient expression in plant leaf protoplasts has been scaled up for use with FACS¹², yet quantitative data are also difficult to obtain due to the large auto-fluorescence signal from abundant chlorophyll, which typically overlaps with signal from the fluorescent proteins used as readouts.

To overcome these limitations, we developed an enhanced throughput transient expression assay in plant protoplasts, using luciferase outputs, to rapidly test the behavior of plant genetic parts. We combine this assay with a rigorous mathematical analysis to account for significant stochastic factors, allowing quantitative analyses of plant parts function. Here, we describe this methodology and demonstrate its use to quantitatively characterize 128 new synthetic plant promoter-repressor pairs. We further show that these parts can be computationally selected and then assembled to produce predictable function in planta.

### Example 1: Building Synthetic Plant Components

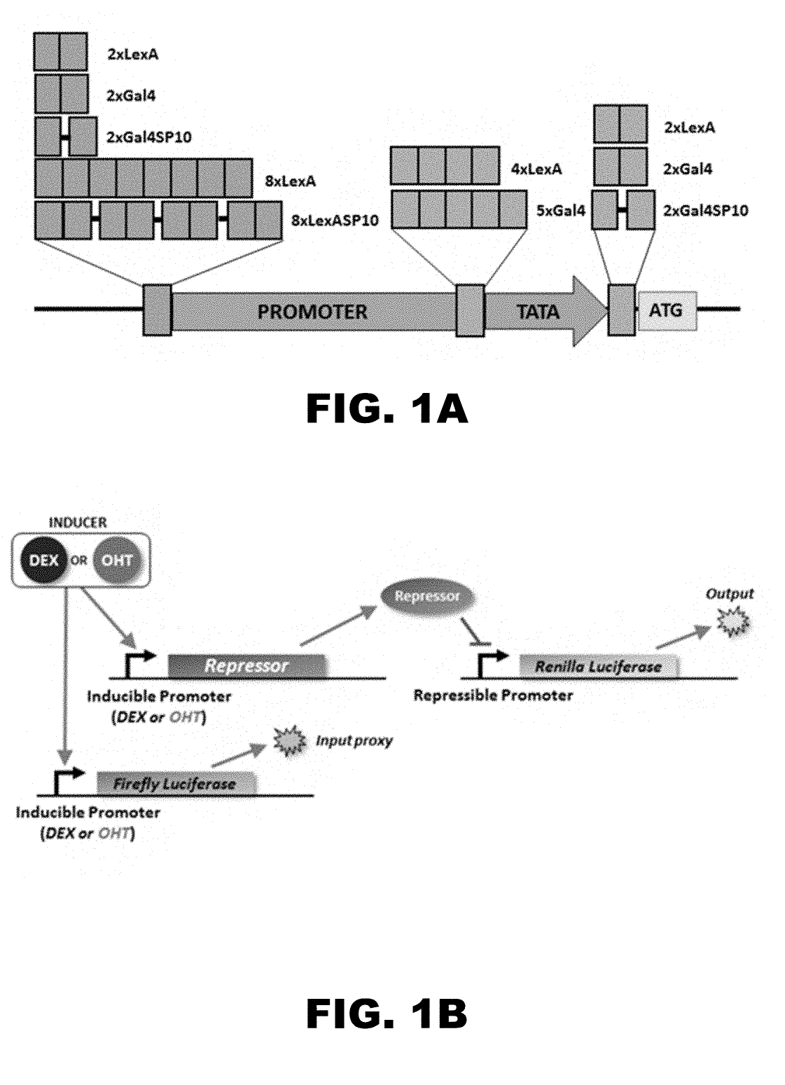

Orthogonality, i.e., the ability of a genetic component to function in an organism independent of endogenous regulation, is an important principle of synthetic biology^{13,14}. To apply this principle, genetic components were chosen with some prior characterization from bacteria, yeast, and plant viruses as sources for engineering synthetic transcriptional repressor proteins and repressible promoters for plants. Synthetic plant transcriptional repressors were created by making translational fusions of the DNA binding domain from the yeast Gal4 or the bacterial LexA transcription factors to previously characterized repression domains found in Arabidopsis repressor proteins (EAR, OFP, BRD)¹⁵⁻¹⁸. Synthetic plant repressible promoters were engineered from promoters naturally found in plant viral and bacterial pathogens [Cauliflower Mosaic Virus (CaMV35S), Figwort Mosaic Virus (FMV), and Nopaline Synthase (NOS)], which have been shown to drive constitutive expression of genes in plants¹⁹⁻²¹. Synthetic promoters were engineered to be both constitutive and repressible in plants by inserting multiple copies of the Gal4 or LexA binding elements (recognized by the above repressors) at specific positions in the CaMV35S, FMV, and NOS promoters. FIG. 1A shows a schematic for how the number, spacing and position of the binding elements (upstream or downstream of the promoter sequence, or just upstream of the TATA-box) may be varied with the goal of producing various levels of binding for the repressor proteins, and hence tunable repression of promoter activity in plants.

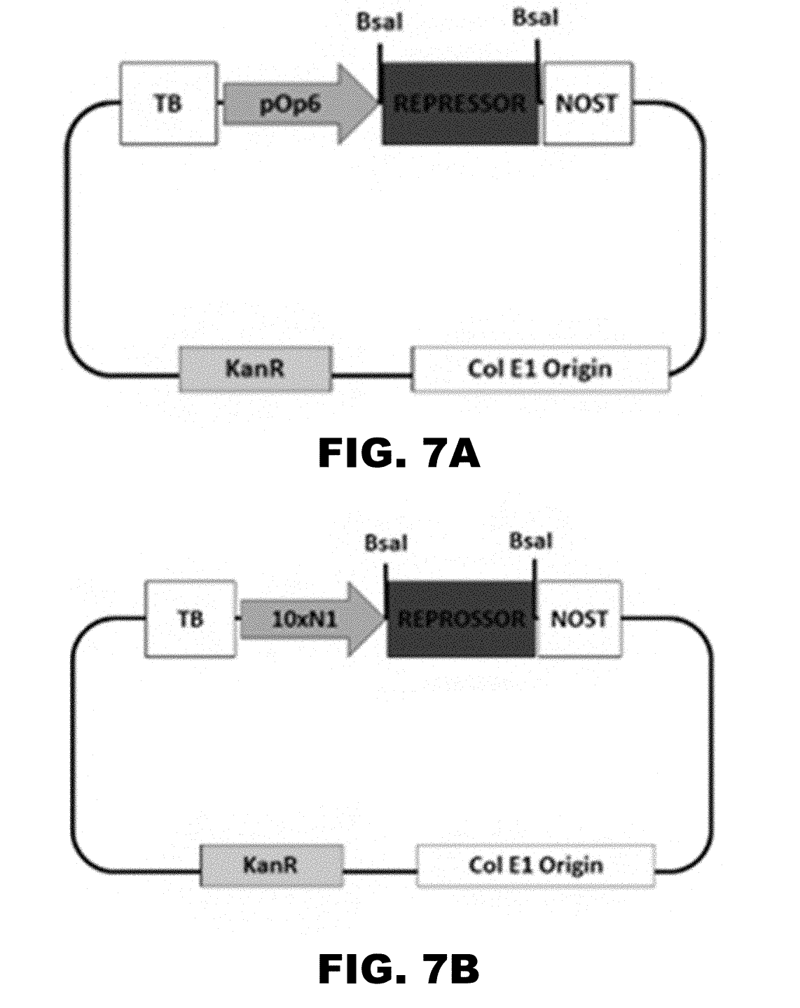

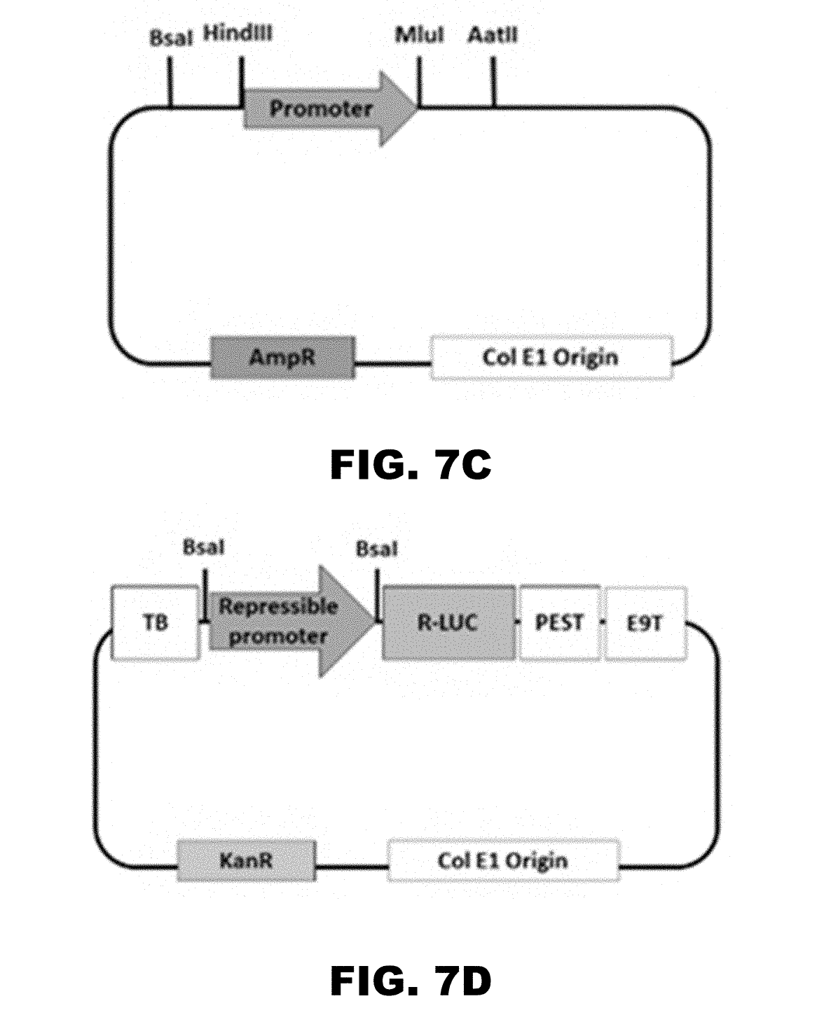

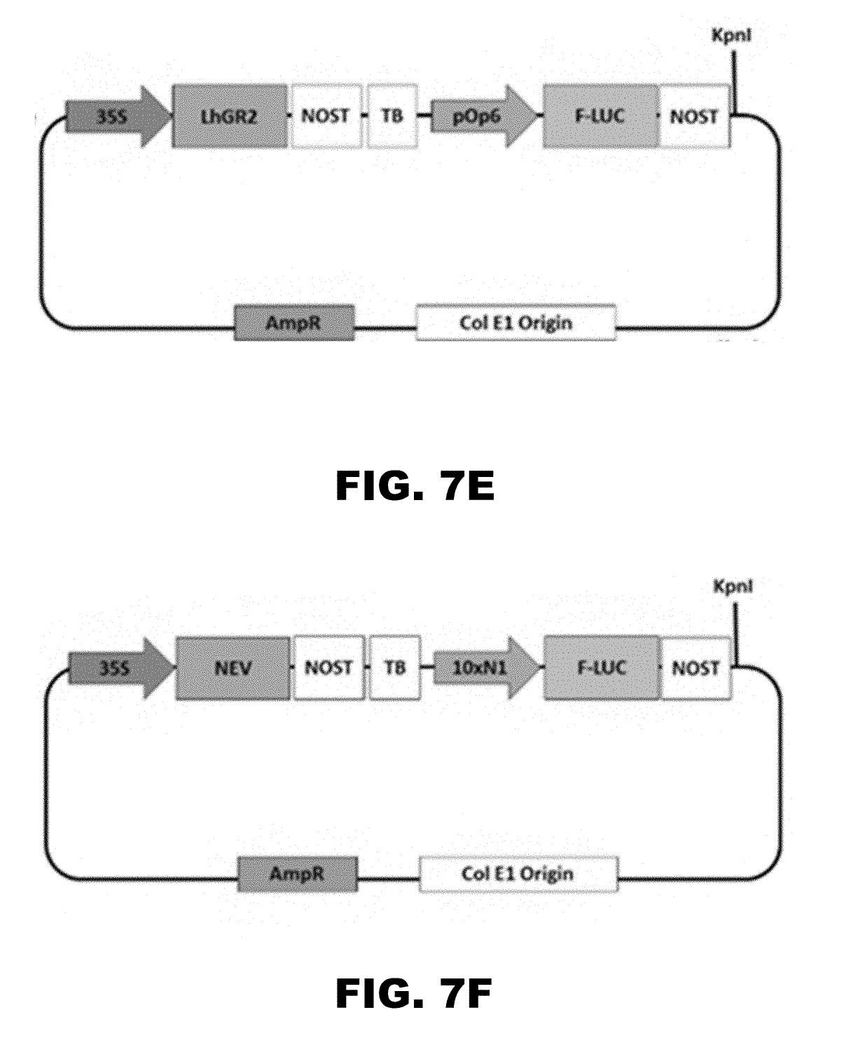

The transcriptional repressor proteins are built out of two genetic components: DNA binding (DB) and repressor domains (RD). The DNA binding domains of the yeast Gal4 and the bacterial LexA transcription factors were used to create orthogonal repressor proteins for the plant system. Ethylene-responsive element binding factor-associated amphiphilic repression (EAR), plant-specific B3 repression domain (BRD), two variants of the Arabidopsis OVATE Family proteins (AtOFP1 and AtOFPx) were used as repressor domains. AtOFPx represents a consensus sequence of the OVATE family repressor proteins demonstrating the highest levels of repression¹⁶. Sequence optimized Gal4 and LexA DB, and the two OVATE RD were synthesized as double stranded gBlocks (GeneArt/Life Technologies and IDT). The synthetic repressor domains were fused in frame to one of two mentioned synthetic DNA binding domains using overlapping extension PCR with compatible BsaI restriction enzyme sites built into the primers for downstream cloning. The small-sized EAR and B3 repressor domains were incorporated into reverse primers used to amplify DNA binding domains, creating in-frame C-terminal fusions. The hybrid products were sub-cloned and sequenced in pJET2.1 vector using pJET forward and reverse primers [Thermo Scientific]. Two core repressor modules containing an upstream transcription block²⁵, estrogen inducible promoters, repressors and NOS terminator²⁶ were synthesized (GeneArt, FIG. 7A-B). To interchange the different repressors in the module, the Golden Gate cloning method²⁷, using type II endonuclease BsaI restriction sites, was used (FIG. 7A-B). Repressor expression was controlled by two inducible promoters, 10×N1 and pOp6, which are 4-Hydroxytamoxifen²⁸ (4-OHT) and Dexamethasone²⁹ (DEX) inducible, respectively. Each of the inducible promoters was also designed to direct expression of Firefly luciferase (F-luc) gene (FIG. 7E-F). F-luc reporter gene serves as a proxy for quantifying the amount of repressor in the system.

The constitutively active repressible promoters were constructed by introducing DNA binding elements (operators) in the backbone of Cauliflower Mosaic Virus 35S (CaMV35S), Nopaline Synthase (NOS) and Figwort Mosaic Virus (FMV) promoters²⁰. The DNA binding elements containing two copies of Gal4, and two or eight copies of LexA, were synthesized as a gene block with appropriate restriction sites included (IDT). A library of repressible promoters was generated by varying the number of DNA binding elements, the spacing between each binding element and its position relative to the transcription start site. Plasmid backbone (FIG. 7C) was used as a sub-cloning plasmid, from which promoter variants were made by adding DNA binding elements upstream and downstream of the promoters. Upstream of each promoter, DNA binding elements were cloned using BsaI/HindIII restriction sites, whereas downstream of the promoters, elements were cloned using MluI/AatII sites. Two CaMV35S based promoters with 4×LexA and 5×Gal4 binding elements at the −32 position were synthesized (GeneArt). For ease of cloning, a single repressible promoter module with two BsaI restriction enzyme sites flanking a repressible promoter was synthesized (FIG. 7D). The synthetic module has a 5′ transcription block, repressible promoter controlling expression of Renilla luciferase (R-luc), a PEST domain³⁰, and the NOS terminator (FIG. 7D). The resulting promoter fragments from sub-cloning vector (FIG. 7C) were digested with BsaI and cloned into the repressible promoter module (FIG. 7D) upstream of R-luc. We use R-luc to quantitatively determine the repressibility of the promoter upon repressor binding. In theory, a functional repressor/repressible promoter pair should show decreasing R-luc activity with increasing F-luc activity as a result of increasing inducer concentrations.

Two pBluescript SK⁺ backbone plasmids (FIG. 7E-F) containing F-luc under the control of two estrogen inducible systems, 10×N1/NEV²⁸ and pOp6/LhGR²⁹, were used to assemble the expression cassette encoding the repressors and corresponding repressible promoters. Plasmid backbones were prepared by restriction digest with KpnI and simultaneously dephosphorylated with alkaline phosphatase (FastAP, Thermo Scientific) to prevent self-ligation. The repressor and repressible promoter fragments were prepared by digesting with BsaI and KpnI. The two sticky BsaI ends from the repressors and repressible promoters are compatible and the two external KpnI sites were used for non-directional cloning into the vector backbone. Similarly, four beta plasmids, i.e., containing repressible promoters controlling expression of R-luc without any repressors, were also constructed to monitor the maximum level of expression of R-luc (strength of the promoter). Electro-competent E. coli strain DH5a was used for all cloning purposes. Primers were synthesized by IDT (Integrated DNA Technologies). PCR reactions were performed using Herculase II fusion DNA polymerase (Agilent Technologies). All restriction enzymes were purchased from NEBioLabs and Thermo Scientific. Plasmid preparations and gel extractions were conducted using Thermo Scientific GeneJET and Zymo Research miniprep and gel purification kits. DNA sequencing was provided by the Colorado State University Proteomics and Metabolomics Facility.

### Example 2: Quantitative Testing of Plant Parts

To quantitatively measure the input-output function of the synthetic repressible promoters, we constructed a test bed by linking each synthetic promoter to Renilla luciferase (R-luc) to provide a direct readout of the promoter's quantitative behavior (FIG. 1B). Expression of the cognate synthetic repressors were then modulated with one of two previously characterized inducible transcriptional control systems, dexamethasone (DEX) and 4-hydroxytamoxifen (OHT)^{22,23}. To simultaneously quantify the repressor, a second copy of the inducible promoter was added to a second reporter, firefly luciferase (F-luc). Hence, F-luc serves as a proxy for the amount of repressor produced (FIG. 1B), as the amount of F-luc (measured by luminescence) and the amount of repressor should be proportional to each other.

Repressible promoters and their cognate repressors were cloned into a single plasmid and transiently expressed in Arabidopsis leaf protoplasts (FIG. 7). A commercially available dual-luciferase system (Promega Co., Madison, Wis.) allows for the measurement of both luciferase readouts in the same protoplast sample. Hence, by varying the concentration of inducers (DEX or OHT), and therefore the amount of repressor in the system, it is possible to measure the input-output relationships by quantifying the production of F-luc and R-luc. To increase assay throughput, we modified an Arabidopsis leaf protoplast transient assay (described further in Example 6) to allow testing of multiple repressor-promoter combinations, with multiple inducer concentrations, in a 96-well plate format.

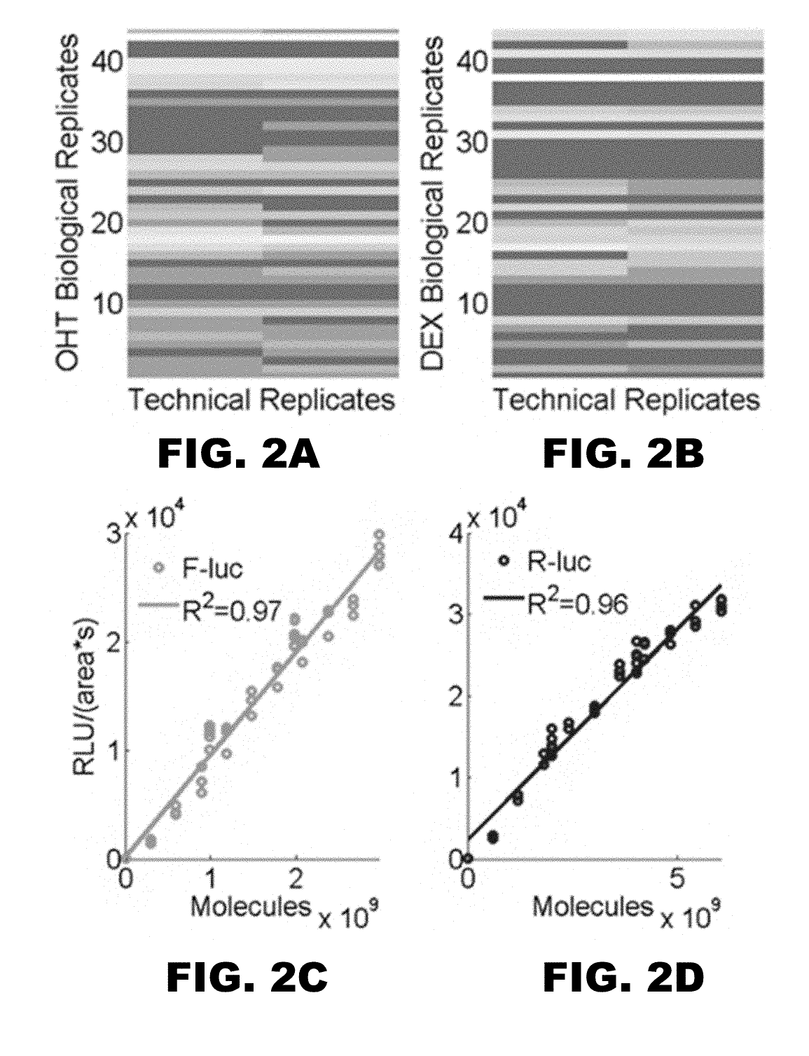

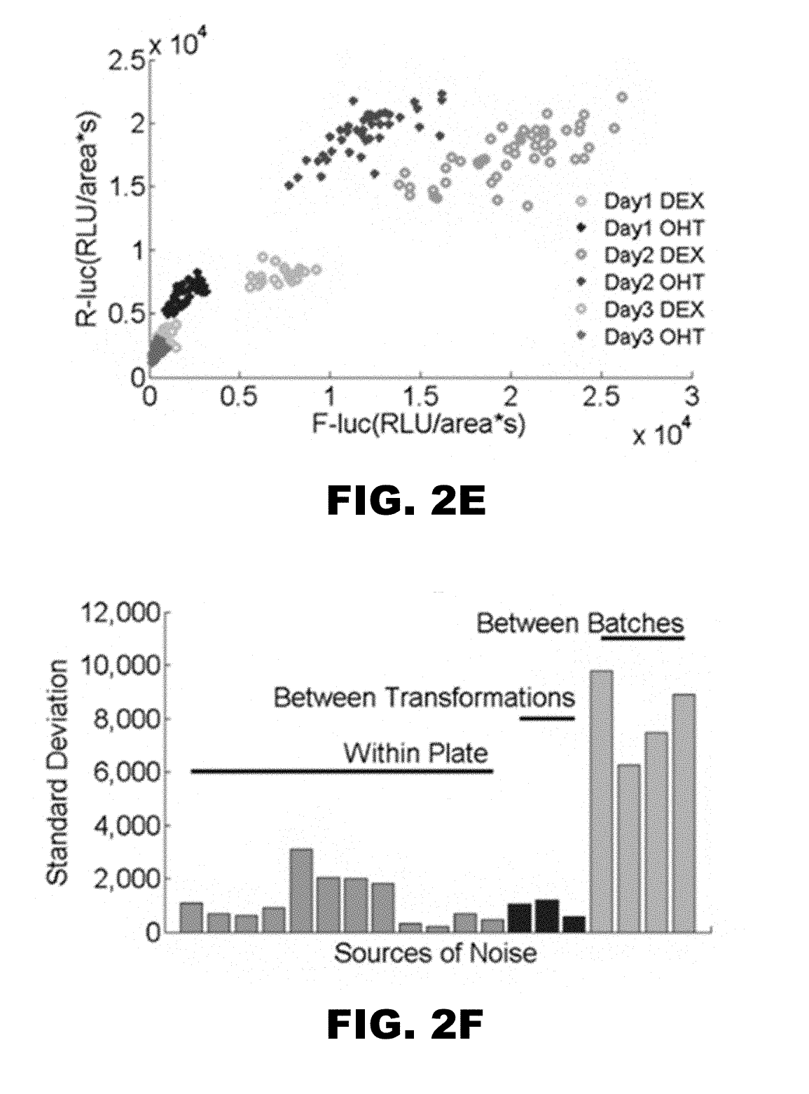

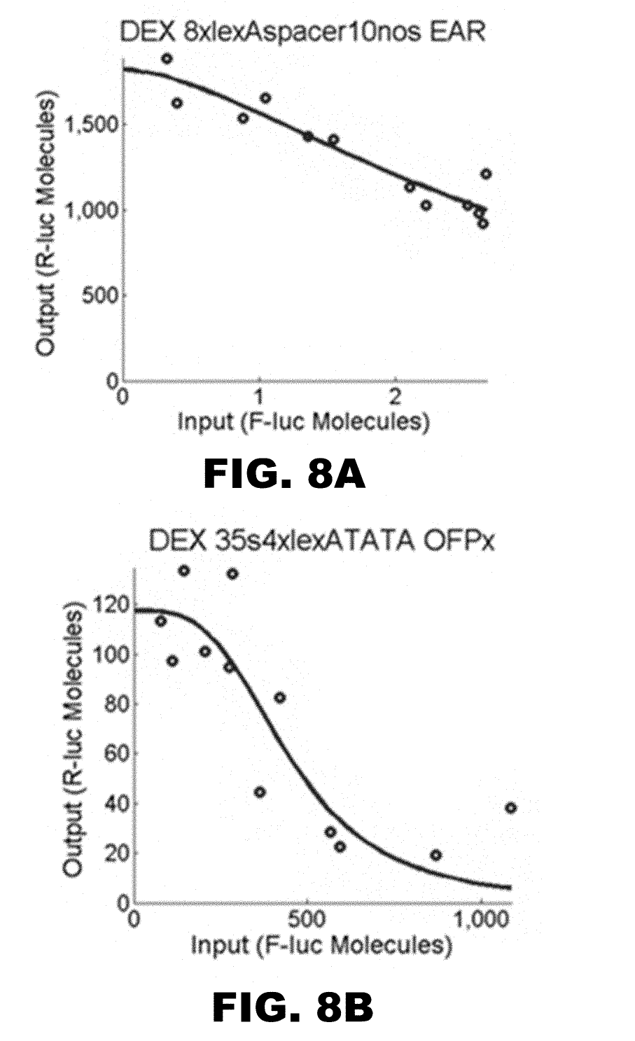

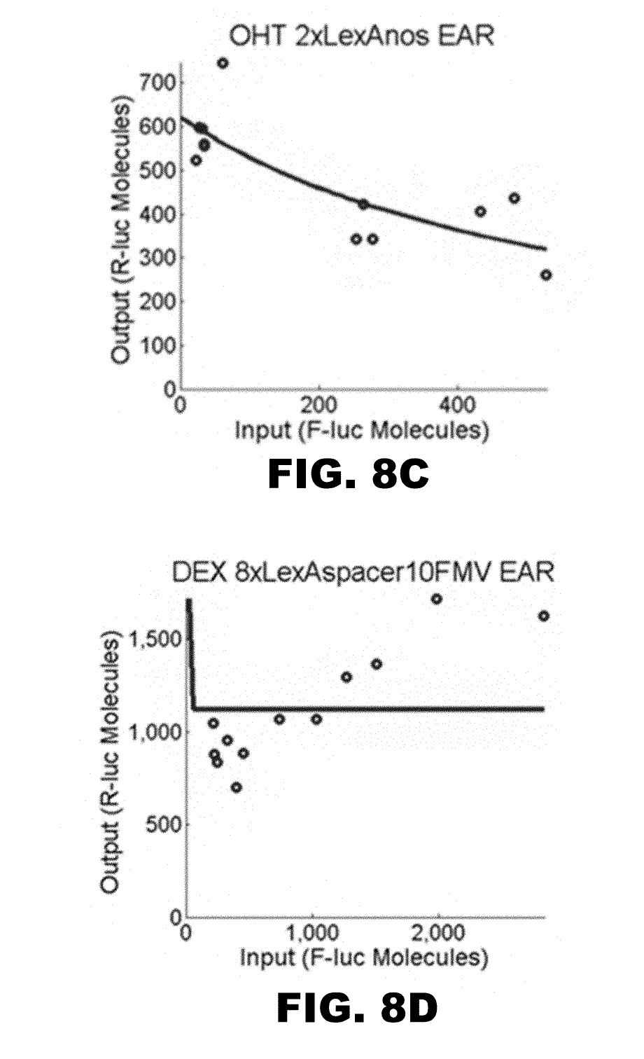

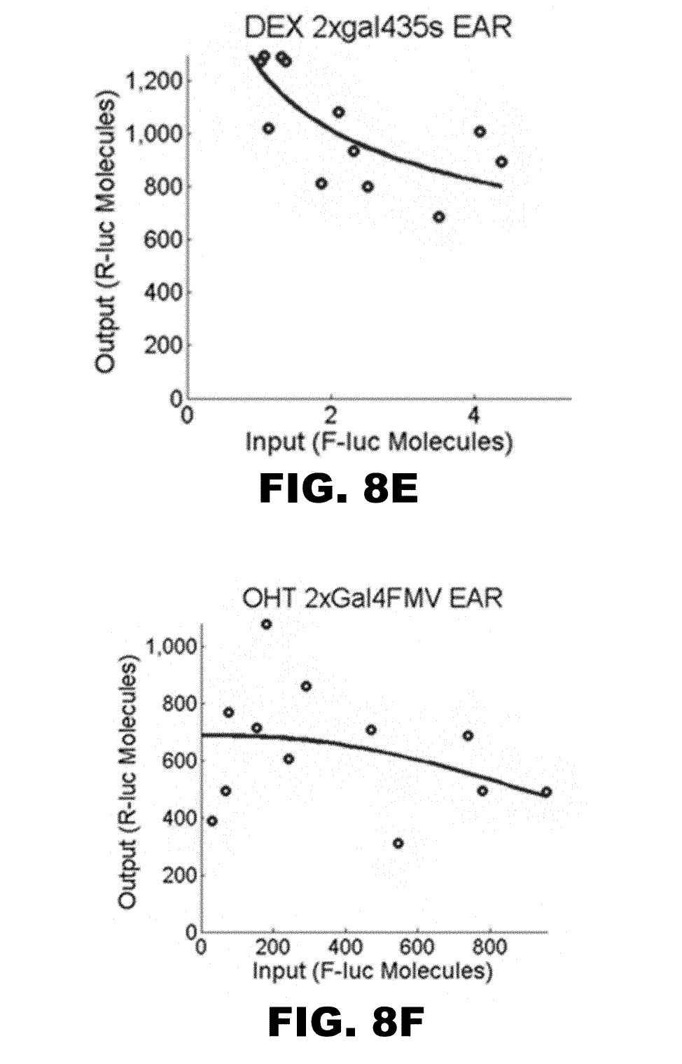

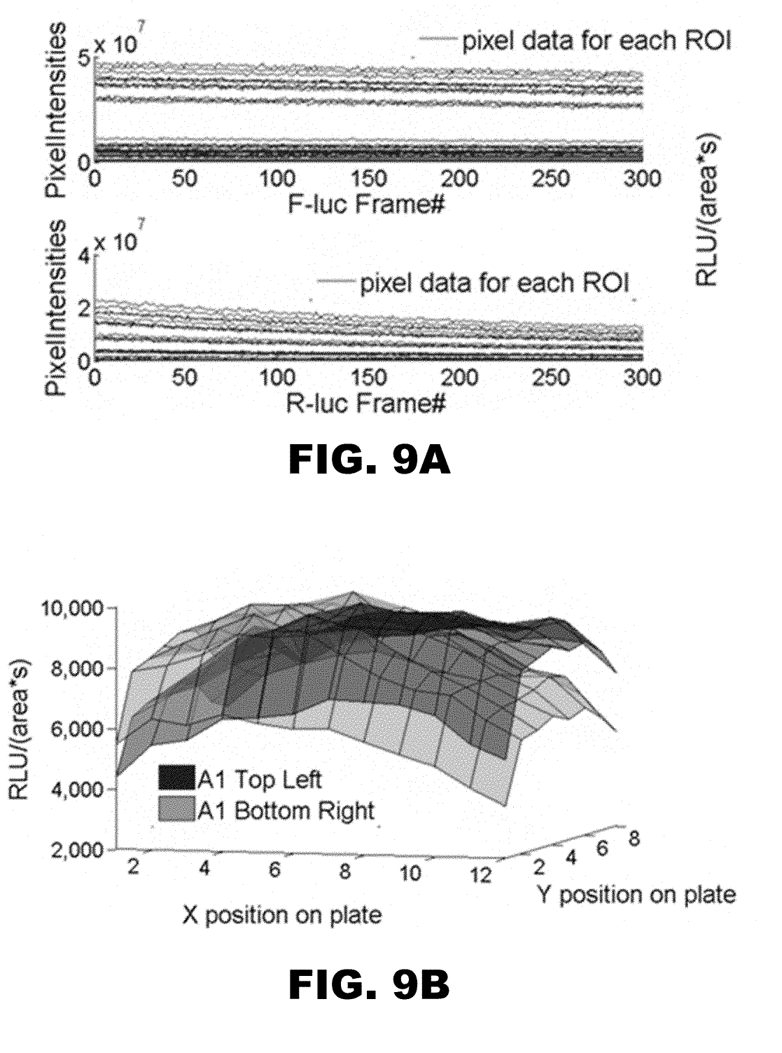

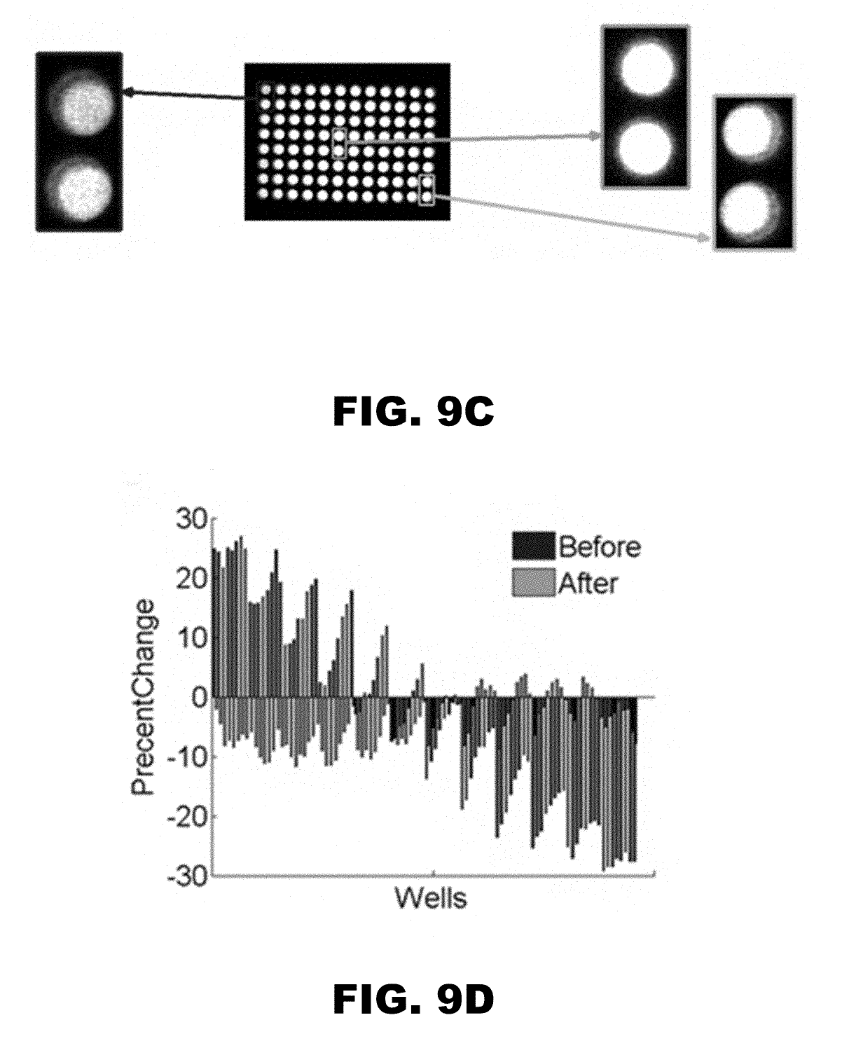

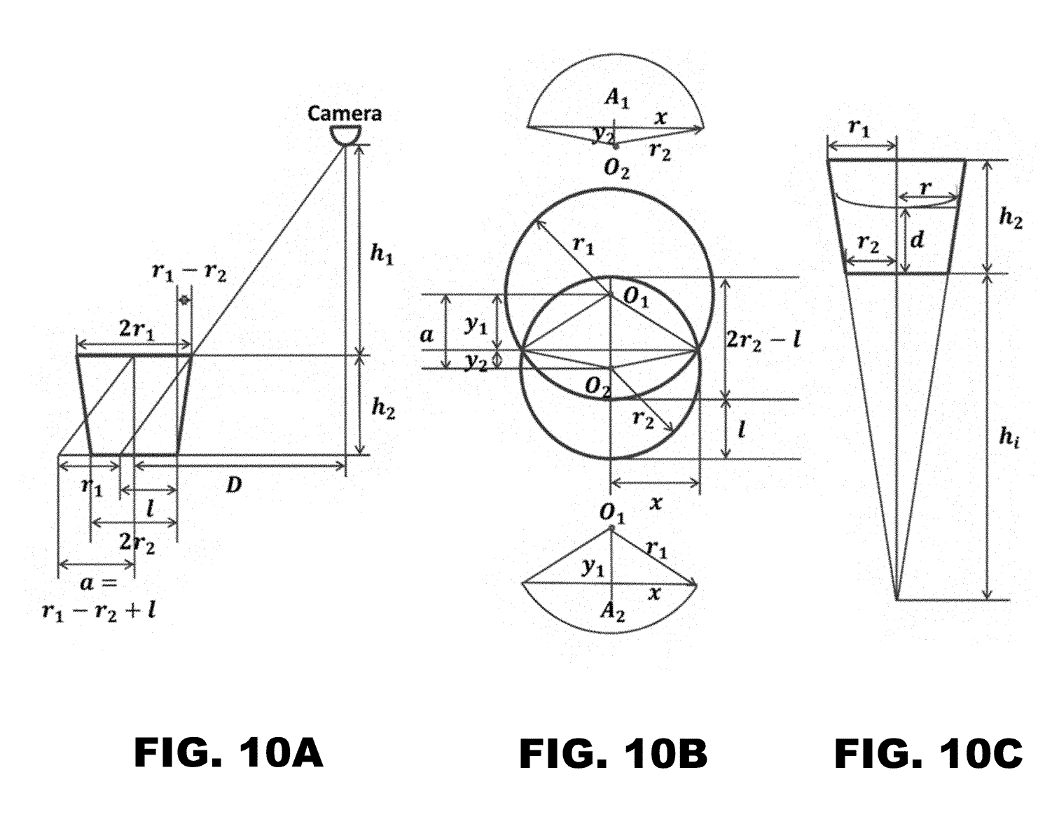

Luminescence of both F-luc and R-luc was measured using a single photon ICCD Camera (Stanford Photonics, Inc.). With increasing inducer concentrations increasing levels of F-luc (input) was observed coupled with decreasing levels of R-luc (output). Initial results from the protoplast experiments showed large variability (noise) in the data (FIG. 8). To see if the data were comparable across different genetic circuits, we the basal level (i.e., without any inducer added) of F-luc luminescence was examined, which should be the same across all experiments in the absence of variability across protoplast batches or transformations. The data are shown as a grey-scale intensity plot in FIG. 2A-B, and displays significantly more variability between plasmids than between technical replicates of the same plasmid. This analysis suggests that the protoplast experiments are subject to significant variability from the different batches of protoplasts, transformation efficiency and different genetic circuits, among other possible effects. Below, the variability from the experimental approach, and methodology developed to mitigate their effects, is addressed. One source of noise in the data was systematic, and found to be related to the data collection method used (single photon imaging camera and luminescence from individual wells of 96 well plates). To correct for these imaging errors, a simple geometric method was developed (see Example 12 and FIGS. 9 and 10).

Luminescence values are typically reported in relative luciferase units (RLUs) within an area over collection time (RLU/area*s). To estimate luciferase activity in physical units, assays with purified recombinant F-luc and R-luc were used to quantify the relation between the luminescence and the number of luciferase molecules (FIG. 2C-D and further discussed in Example 12).

### Example 3: Analysis of Stochastic and Experimental Variations

There are several possible sources for the variability we observe in FIG. 2A-B. From the experimental design, we expect three sources to make the major contributions to noise in our system: (1) within-plate variation, i.e., random variation arising from assay procedures, such as pipetting the cells while plating; (2) between-transformation variation, i.e., variation arising from processes involved in protoplast transfection, such as possible variations in transformation efficiency from different plasmids; and (3) between-batch variation, i.e., variation arising from processes involved in preparing each batch of protoplasts for further experiments, including but not limited to variations in the health of the leaf tissue used, effects on protoplast from shear stress during various pipetting and centrifugation steps, slight variations from enzymatic sources.

Before rigorous mathematical models for gene function could be developed, experiments were designed to isolate these three potential sources of noise and determine their actual experimental magnitude. A plasmid that contains all elements found in the promoter-repressor test system were used, except the repressor, and prepared protoplast transformations with one DEX-inducible gene circuit and one OHT-inducible gene circuit. Luminescence data was collected with no inducer added, and the experiment was repeated on three different days. In the absence of any noise, all wells should display identical F-luc and identical R-luc luminescence.

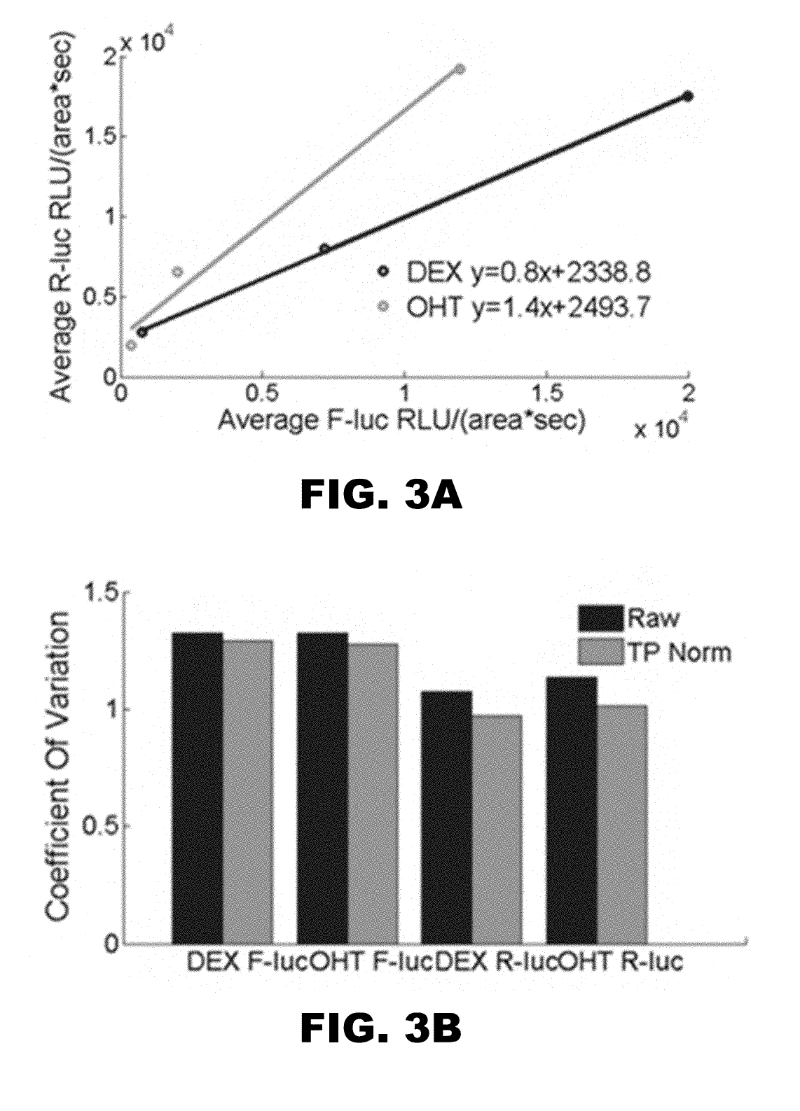

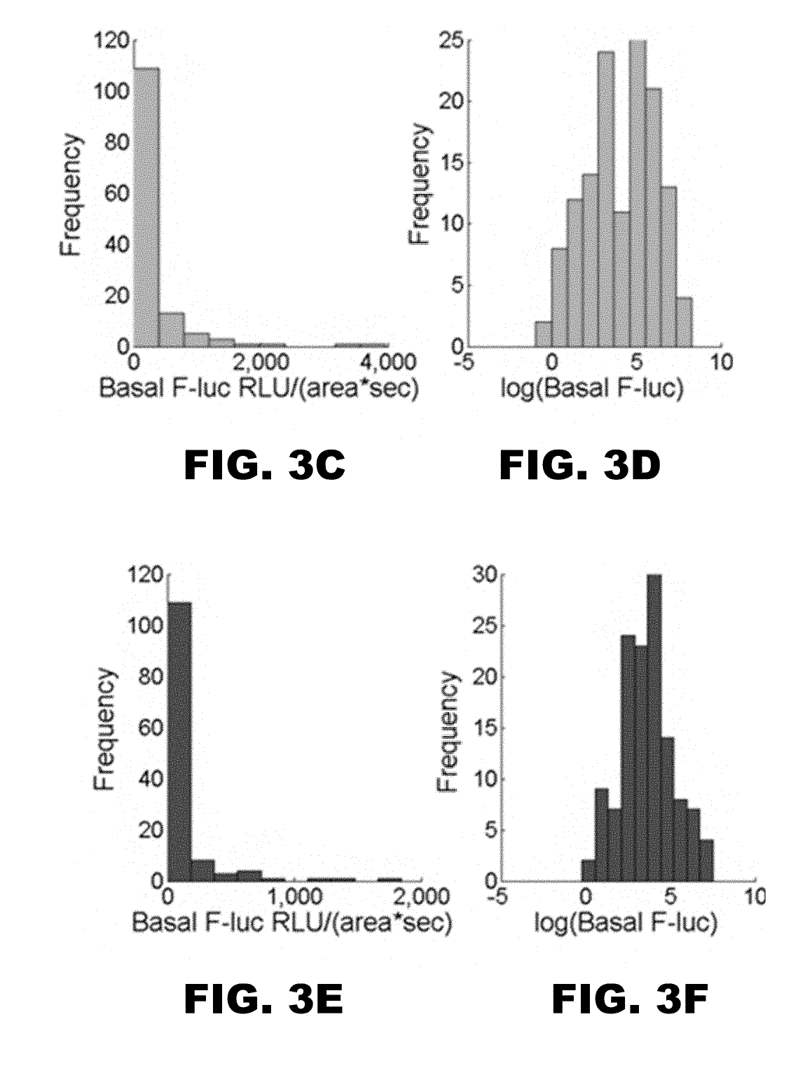

The most apparent source of noise in the data originates from distinct batches of protoplasts prepared on different days (FIG. 2E). Luminescence values of each batch form distinct and well-separated clusters, and variations within each cluster are smaller than the variations between clusters. The magnitude of noise from each of these three sources is calculated and shown in FIG. 2F, which confirms that the most variance comes from between-days preparation of protoplasts, referred to herein as the batch effect. If the batch effect can be represented by a random multiplicative factor that is the same for both F-luc and R-luc luminescence, it was expected that a plot of the average R-luc and F-luc luminescence against each other should be linear. FIG. 3A shows a plot of these data displaying a strong linear trend, supporting the hypothesis that the noise between batches is multiplicative (see details in Example 12).

While the data clearly show that variation from preparation of protoplast batches is the greatest source of noise, the origin of this noise has not been precisely identified. Because the protoplasts are prepared from leaves, each day's preparation likely contains distinct compositions of differentiated cells within a leaf (e.g., mesophyll, palisade parenchyma, bundle sheath) produced from plants that experience micro-climatic variations within our growth chambers. While all protoplasts are pooled and treated equally, the data represent a bulk measurement of all protoplasts in an individual well. As such, different protoplast cell populations may be represented in each day's preparations, giving rise to the random batch effect. Based on the above analysis, a quantitative model of the protoplast data to test the input-output characteristics of the repressor-promoter pairs was constructed.

### Example 4: Mathematical Model and Methodology to Normalize Batch Effects

To develop a model, the experimental data were first defined with mathematical symbols as follows. In all cases the indices ij refer to the j-th well of the i-th plasmid. Concentrations are in molecules per well and RLU units are RLU/(area*sec).

1. Concentration of repressor=Rᵢⱼ

### 2. Concentration of R-luc=[Rluc]ᵢⱼ

### 3. Concentration of F-luc=[Fluc]ᵢⱼ

4. R-luc luminescence in RLU units=Lᵢⱼ
5. F-luc luminescence in RLU units=Fᵢⱼ

It was desired to determine the quantitative relationship between the concentration of the repressor and the expression of R-luc as driven by the constitutively active repressible promoter. It was assumed this relationship is represented by a Hill function^{3,24}. Hence, for a single plasmid in a protoplast:

$${[ Rluc ]}_{ij} = \frac{{β}_{i}}{1 + {( \frac{{R}_{ij}}{K} )}^{n}} . \qquad 1 )$$

Here βᵢ represents the maximal expression of the R-luc protein in the absence of the repressor, while K is the concentration of repressor required for half-maximal expression of R-luc, and n is the Hill coefficient. We assume that every protoplast batch has associated with it a batch effect multiplicative factor, α. Further, we assume that each well has Nᵢⱼ plasmids. Then the total R-luc luminescence in the j-th well of the i-th transformation is related to the concentration of R-luc by Lᵢⱼ=C₂αᵢNᵢⱼ[Rluc]ᵢⱼ where C₂ is the slope of the R-luc standard curve.

Despite being controlled by the same promoter, the concentrations of F-luc and the repressor are not necessarily identical, but should be linearly proportional to each other. For a single plasmid therefore we can write [Fluc]=\tilde{C}R, where \tilde{C} is the proportionality constant. Generalizing again to the j-th well of the i-th transformation, the F-luc luminescence can be expressed as: Fᵢⱼ=C₁αᵢNᵢⱼ\tilde{C}Rᵢⱼ, where C₁ is the slope of the F-luc standard curve.

Now a Hill functional form was fit between the variables Lᵢⱼ and Fᵢⱼ. This function is written as

$$\frac{{B}_{i}}{1 + {( \frac{{F}_{ij}}{{H}_{i}} )}^{n}} \qquad 2 )$$

The parameter Hᵢ represents the half-maximal whole-well R-luc luminescence, while the parameter Bᵢ represents the whole-well R-luc luminescence of the i-th plasmid. In terms of the parameters for a single plasmid therefore, a best-fit estimate of Bᵢ is given by Bᵢ=C₂ [symbol] Nᵢⱼ [symbol] αᵢβᵢ where the angled brackets indicate a mean over the j-index, i.e., over the wells in each plasmid. Similarly, Hᵢ=C₁αᵢ [symbol] Nᵢⱼ [symbol] \tilde{C}Kᵢ.

Though the estimated parameters are proportional to the single promoter parameters β and K, they also contain the unknown multiplicative batch effect term α, which complicates comparison of the repressible promoter strength β, between plasmids. Thus, we considered normalization methods that remove or reduce the effect of this parameter on our data. We first tested the possibility that the batch effect can be removed by normalization with the total protein content of the wells. FIG. 3B shows the effect of normalization by total protein measurements from two separate protoplast batches, showing at best a minor difference between the variability of raw versus normalized data.

It was hypothesized that the factor controlling the variation from different batches of protoplasts, α, is related to the overall biological health of the protoplasts. Therefore, the term αᵢNᵢⱼ describes the number of plasmids in viable protoplasts in the j-th well of the i-th transformation. We define γᵢ as the basal expression of the inducible promoter, which is a constant since the inducible promoter that controls F-luciferase is exactly the same on all gene circuits (i.e., the same for all gene circuits induced by OHT and the same for all gene circuits induced by DEX). Hence, the luminescence of F-luc without inducer, Fᵢⱼ⁰, can be described by Fᵢⱼ⁰=C₁αᵢNᵢⱼ⁰γᵢ and the distribution of its values for all i and j is proportional to the distribution of αᵢNᵢⱼ⁰, where the 0 superscript refers to the wells with zero inducer. FIG. 3C-D shows a histogram of the distribution of Fᵢⱼ⁰, which is also plotted on a log-scale in FIG. 3E-F. The distribution of Fᵢⱼ⁰ is fit quite well by a log-normal distribution, suggesting a multiplicative source for the batch variation. According to the above analysis, it is possible to mathematically define a normalization factor λᵢ, as follows:

$${λ}_{i} = \frac{{〈 {F}_{ij}^{0} 〉}_{j}}{{〈 {F}_{ij}^{0} 〉}_{ij}} = \frac{{〈 {N}_{ij}^{0} 〉}_{j} {α}_{i}}{{〈 {N}_{ij}^{0} 〉}_{ij} 〈 {α}_{i} 〉} \approx \frac{{α}_{i}}{〈 {α}_{i} 〉} . \qquad 3 )$$

Here it is assumed that the distribution of Nᵢⱼ and αᵢ are independent of each other and that the distribution of Nᵢ₀ is sharply peaked, so that [symbol] Nᵢ₀ [symbol] ≈ [symbol] Nᵢ₀ [symbol] ᵢⱼ. Next, we divide every value of F-luc and R-luc luminescence for the i-th plasmid by the normalization factor λᵢ, which replaces the batch effect factor αᵢ by its mean [symbol] αᵢ [symbol] . The variance in our data due to batch effects is therefore expected to vanish, and is replaced by a constant.

Fitting the relevant non-linear function to the normalized data now estimates Bᵢ=C₂ [symbol] Nᵢⱼ [symbol] [symbol] αᵢ [symbol] βᵢ, Hᵢ=C₁ [symbol] αᵢ [symbol] [symbol] Nᵢⱼ [symbol] \tilde{C}Kᵢ and the Hill coefficient n. This procedure allows us to rank the different promoters and compare them. The single-plasmid parameters βᵢ (promoter strength) and Kᵢ (the repressor concentration at half-maximum) could also be estimated if required by measuring the value of the different constants in the expressions above.

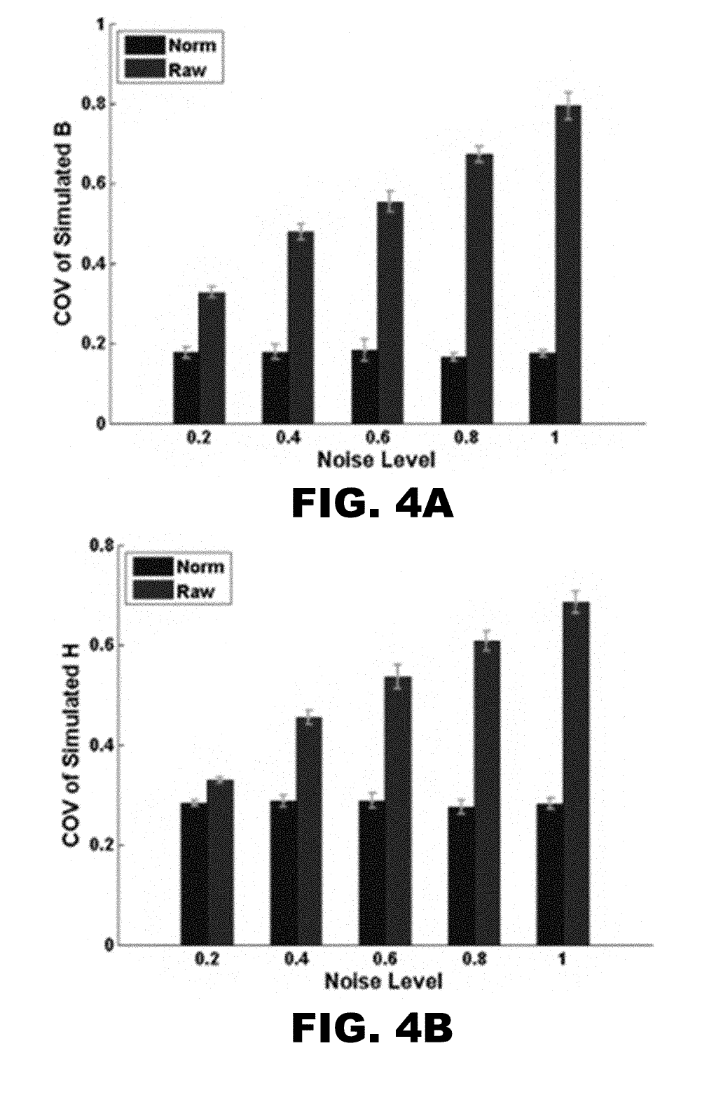

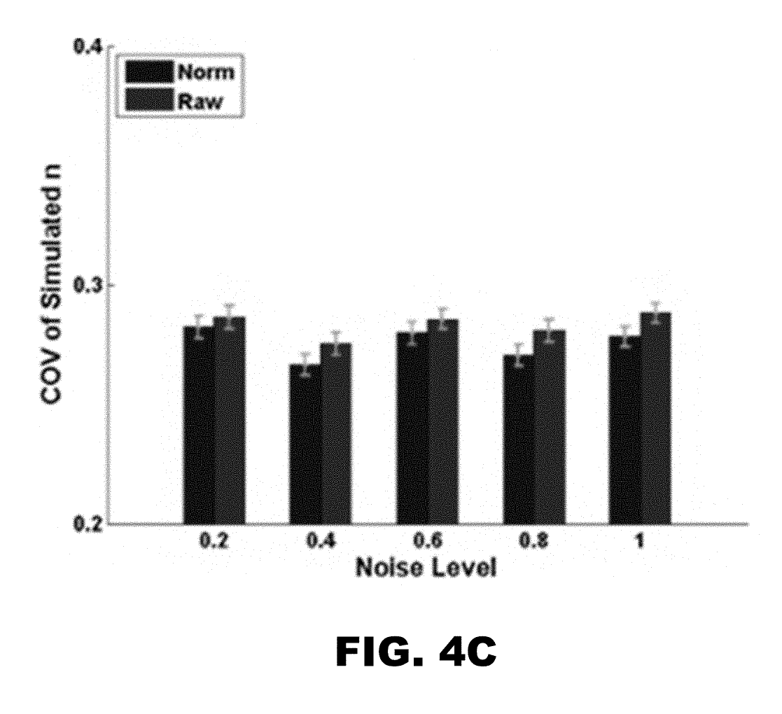

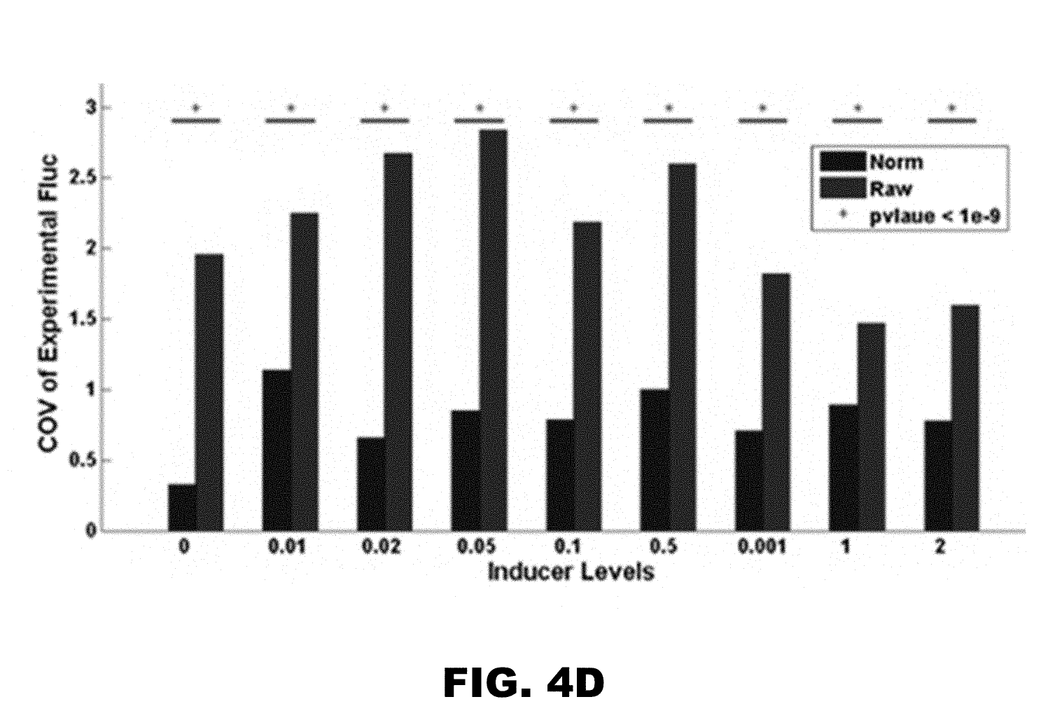

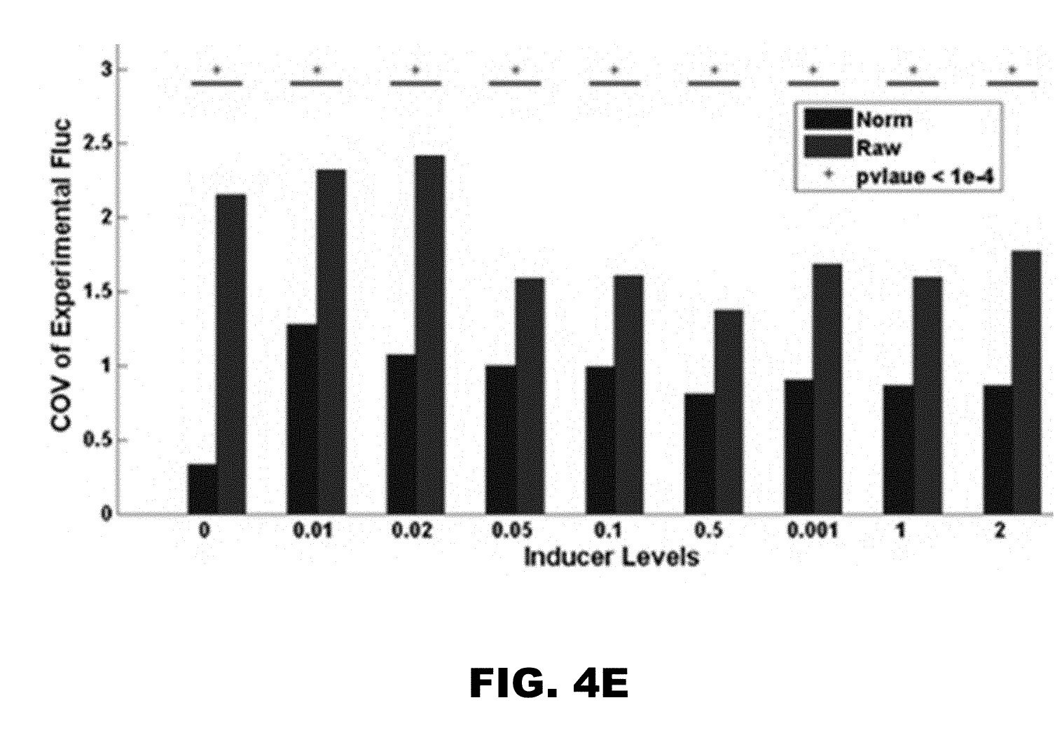

Tests of the normalization method against simulated data (FIG. 4 and Example 12) shows that it reduces the variability in the estimation of the parameters Bᵢ and Hᵢ, and makes them more comparable across plasmids. To test whether the normalization similarly reduced variability in the experimental data, we reasoned that since it reduces the variation between batches, it should lead to a decrease in the noise in the F-luc luminescence at every concentration of the inducer DEX or the inducer OHT that was used in the experiment. Since, as shown in FIG. 1, this part of the genetic circuit is identical between all DEX plasmids and between all OHT plasmids, the major share of the variation in the data across all plasmids should come from the variation due to different protoplast batches. As shown in FIG. 4D-E, for every inducer value the normalized data have a significantly smaller coefficient of variation than the raw data, suggesting that the normalization method does indeed eliminate the batch effect.

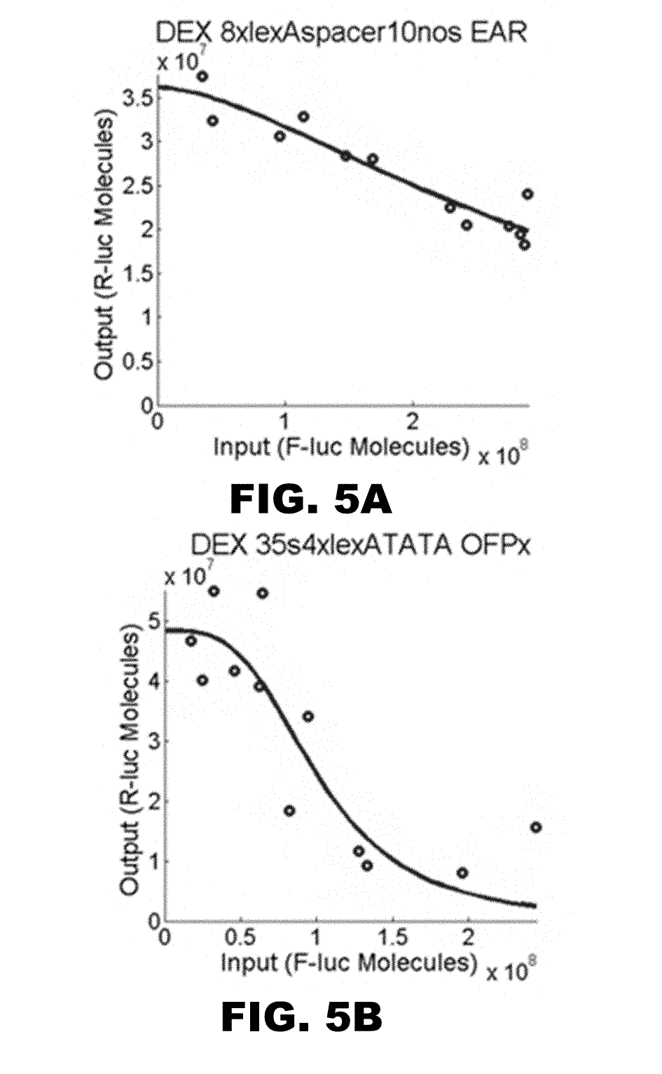

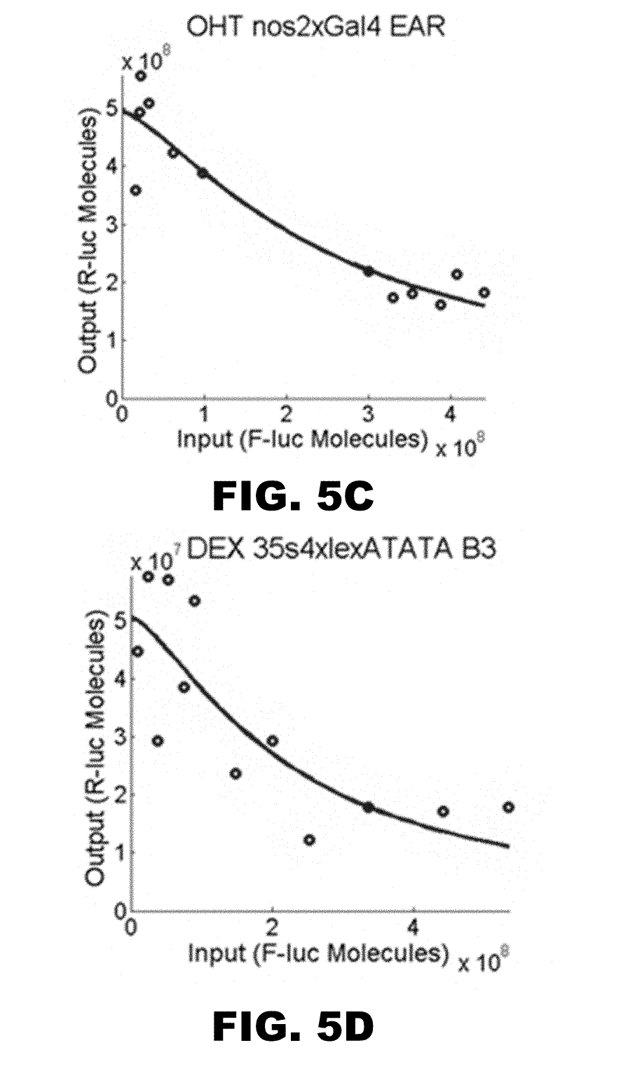

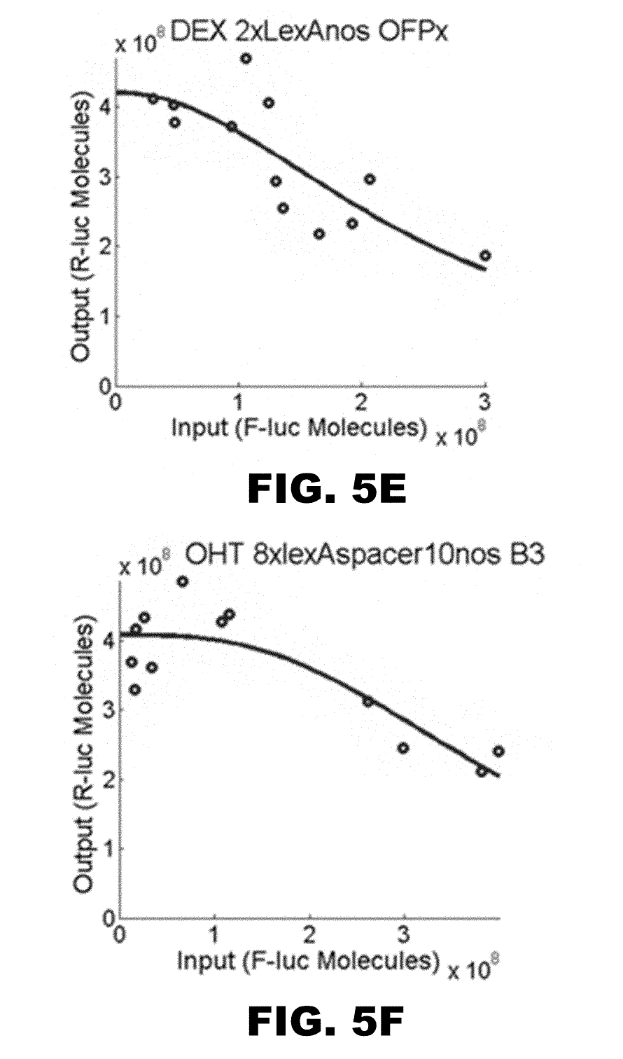

We fit the normalized data using the nonlinear least squares package in Matlab. We tested alternative methods of fitting but found that they did not improve analysis and predictability (data not shown). We also implemented a number of quality control steps to remove assays that appeared to have failed, or promoters that did not work properly (detailed in Example 12). From about 120 gene circuits tested in Arabidopsis protoplasts, about 20 of them met all the criteria. Out of these 20 we selected only those promoter-repressor pairs whose fits had Hill coefficients that were significantly different from zero with a p-value of 0.1 or less for further use. FIG. 5 shows representative fits of the best repressors and promoter pairs from our experimental set.

### Example 5: Testing Predictions in Stably Transformed Plants

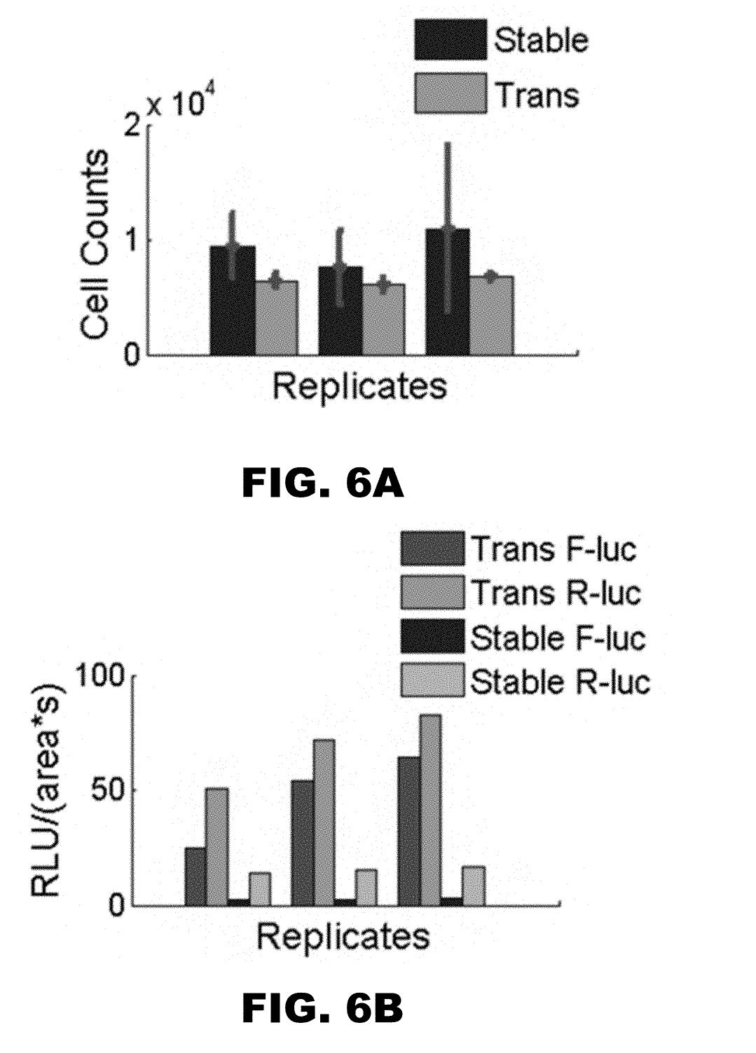

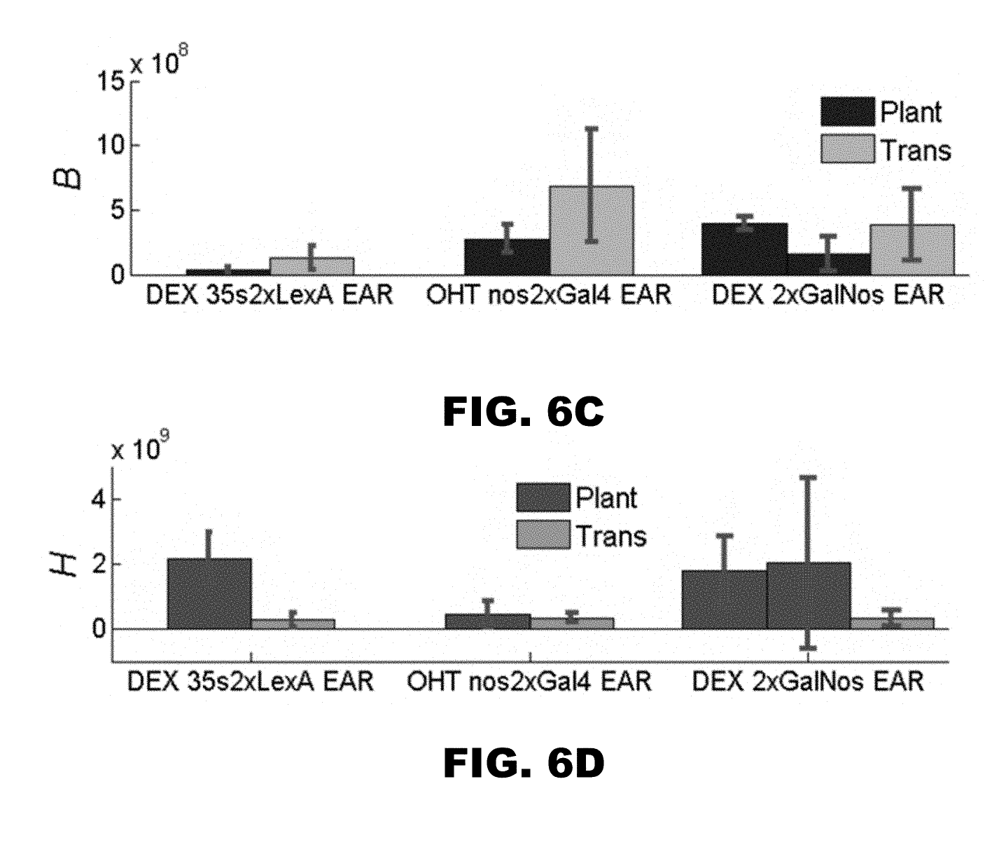

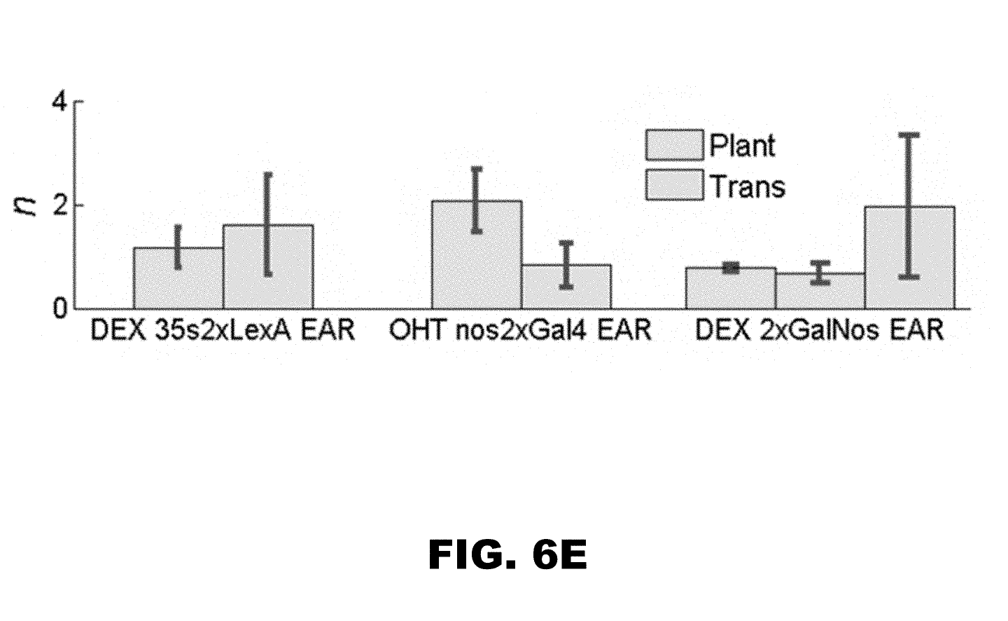

Protoplast assays are useful only if they provide quantitative data that is a reliable measure of the circuit performance in stably transformed plants. To test whether our predictions are a reliable guide to the performance in transgenic plants, we compared the predictions of repressor characteristics in the transient assay with their performance in stably transformed Arabidopsis μlants. Protoplasts were prepared from both wild-type Arabidopsis μlants and transgenic plants stably transformed with a single copy of the repressible promoter circuit described above. Wild-type protoplasts were transiently transformed with the plasmids containing the circuits and induced with increasing concentrations of OHT or DEX. Transgenic protoplasts were also induced with OHT or DEX. F-luc and R-luc luminescence were measured, and parameters were derived using the mathematical analysis described above for both types of protoplasts. The data were normalized using a slightly different method in order to accurately compare the parameters obtained from stably transformed plants with those obtained from transient assays, since the number of working circuits each protoplast contains is significantly different in the two cases (FIG. 6A-B and Example 12).

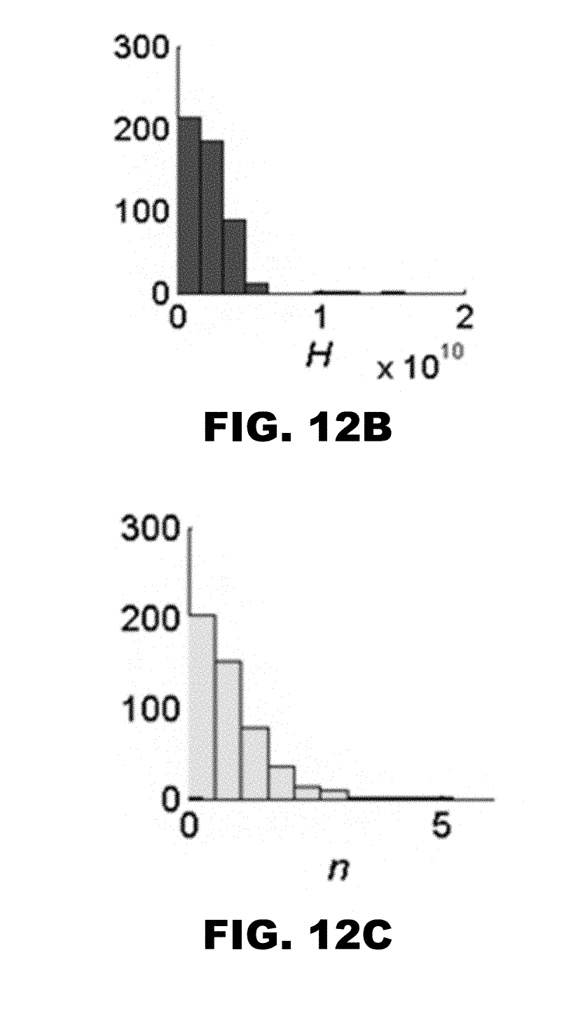

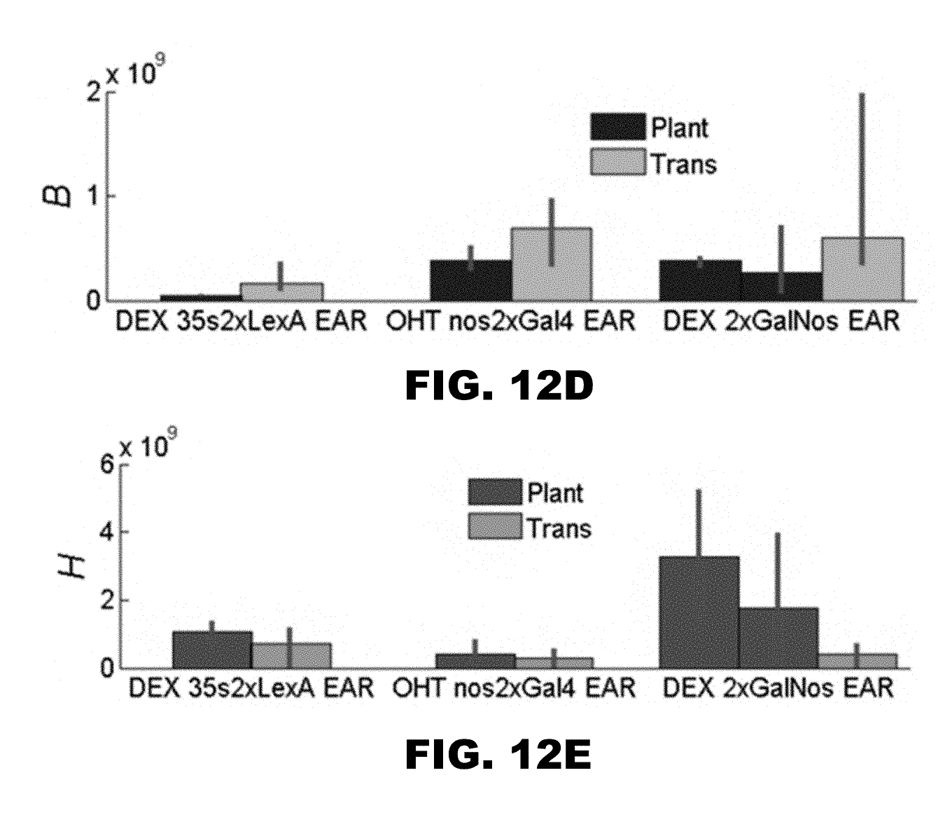

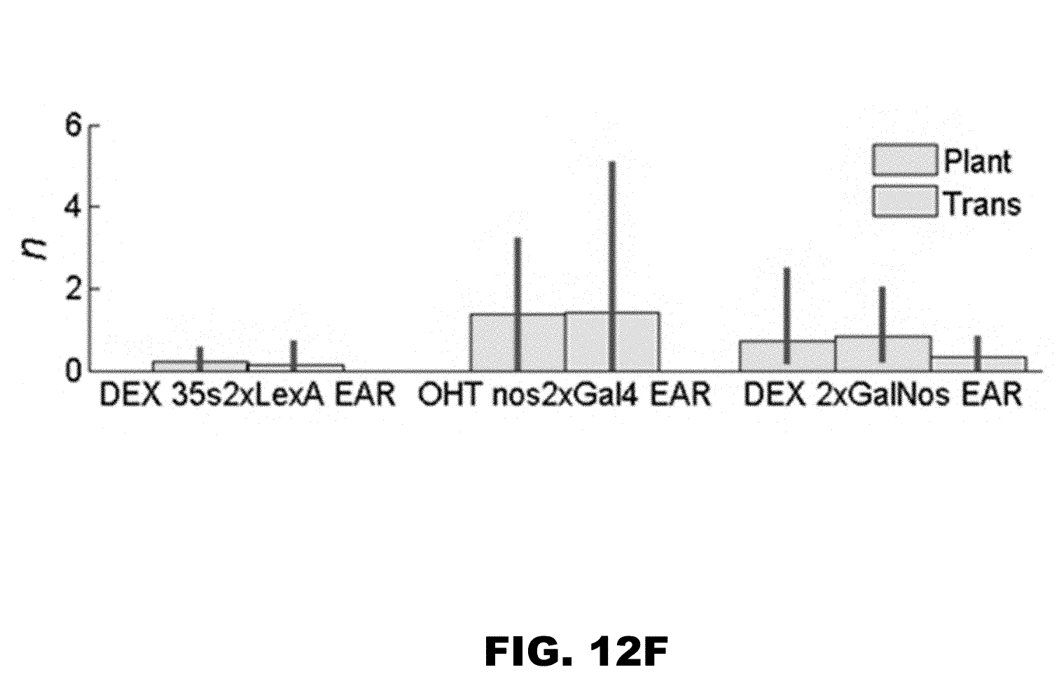

Comparison of the parameters from the normalized data for the stable transgenic plants and for the transient assay are shown in FIG. 6C-E. The predictions for promoter strength B are quite accurate in one of the three cases, and overestimated only by a factor of three or four in the other two cases. The predictions for half-maximal expression H are very good in one of the three cases and within a factor of five for the others. Predictions for n also lie within a factor of two or three of the value for the plant data. Note that in most cases the experimental error bars obtained from 3 repeats of the experiments overlap with the values calculated for the stably transformed plants. Since these experiments are time consuming and it was not possible to perform more repeats, we carried out a bootstrapping statistical analysis to generate mean values and confidence intervals for the predictions. The analysis, shown in FIG. 12, confirms the reasonable agreement between the results for the transient assay and the stable transgenic plants.

### Example 6: Protoplast Isolation and Transfection

Arabidopsis protoplast isolation and transfection were carried out according to the protocol described by Yoo et. al.³¹, with some modifications to allow higher throughput testing of synthetic components in 96-well plates. Wild type Columbia plants were grown in short days (10 h light/14 h dark), and 20-25 leaves, approximately 4 cm in length, were used. In brief, leaves in W5 solution were cut into ˜1 mm strips using a scalpel blade. Enzyme solution [0.4 M Mannitol, 20 mM KCl, 20 mM MES (pH 5.7), 1.5% Cellulase R-10 (Yakult Honsha), 0.4% Macerozyme R-10 (Yakult Honsha), 10 mM CaCl₂, 1 mg/ml BSA] was added, a slight vacuum was applied, and incubated at room temperature with gentle shaking (40 rpm) for 3 hours. Resulting protoplasts were filtered through a 70 μm cell strainer (BD Biosciences) and harvested by centrifugation at 600×g. After two washes in W5 solution, the protoplasts were resuspended in MMg solution, and the concentration adjusted to 2×10⁵ protoplasts/ml. Protoplast transfection with plasmids of interest was performed in 15-ml conical centrifuge tubes by carefully mixing 50 μl of protoplasts (approx. 10,000 cells), 5 μl of plasmid DNA (1 μg/μl), and 55 μl of 40% PEG solution for one reaction. Larger-scale (14 reactions) transfections were used to allow testing of multiple concentrations of inducers. Transfected protoplasts were resuspended in 200 μl of WI solution/reaction, and plated on black, clear-bottom, 96-well Costar assay plates (Corning), using a multi-channel pipette. Inducers (4-OHT or dexamethasone) were added also using a multi-channel pipette, and plates were incubated overnight in the dark, with gentle agitation (50 rpm).

### Example 7: Luciferase Imaging

All test plasmids used in this work had Firefly and Renilla luciferases as the measurable outputs. Therefore, the Dual-Luciferase Reporter Assay system (Promega) was used to lyse the protoplasts and provide both substrates for luciferase imaging. After overnight incubation of protoplasts with the inducers, cell lysis was carried out on the assay plates by removing 160 μl of supernatant from each well, followed by addition of 50 μl of 2× Passive Lysis Buffer, and incubation at room temperature for 30 minutes. Quantitative measurements of Firefly and Renilla Luciferase expression were obtained by the addition of LAR II and Stop & Glo reagents, respectively, and imaged using a Stanford Photonics XR/Mega-10 ICCD Camera System and available Piper software (v. 2.6.17). Regions of interest, ROI's, are drawn around each well of a 96-well plate. Pixel intensity values for the 1^{st} minute of collection time are summed and divided by the area of the ROI and time collected to give us the RLU/(area*s) value in each well. The data then go through post-image correction (below).

### Example 8: 96-Well Plate Post-Image Correction

Five primary systematical parameter values were first determined: 1) r₁ (Radius of well opening); 2) r₂ (Radius of well bottom); 3) h₁ (Height of the camera relative to the surface of the 96 wells); 4) h₂ (Depth of well); 5) V (Reaction reagent total volume). These parameters were then fed into the function V(d) (Example 12) to yield the secondary parameter d (depth of solution added in the well). Next, values of r₁, r₂ and h₁ were substituted into function Aᵥ (Example 12) resulting in Aᵥ (s,D). Then h₂ and d were substituted as the lower and upper integration limits, respectively, for the integration of Aᵥ(s)ds, resulting in Vᵥₜₒₜₐₗ(D). Here, the function is fully parameterized and the only input needed is D, the positional parameter corresponding to each well in this algorithm. It was assumed the well in the i^{th} row, j^{th} column from the top left corner of the microplate (as the origin of 96-well-plate plane) holds a coordinate of (xᵢⱼ,yᵢⱼ) and the projected camera center onto the plate holds a coordinate of (x,y). Then, Dᵢⱼ for the well (i, j) can be calculated as Dᵢⱼ=\sqrt{(xᵢⱼ−x)²+(yᵢⱼ−y)²}. Substitute Dᵢⱼ into Vᵥₜₒₜₐₗ(D) to generate the total visible volume for well (i, j). The ratio of this total visible volume to V (total reagent volume) is then used for camera correction.

### Example 9: Noise Estimation

The noise measurements shown in FIG. 2F are calculated as follows. For within-plate noise (source 1) the standard deviation of F-luc and R-luc luminescence for each plate independently was calculated. This gives a measure of between-well noise for a single plasmid on a single plate. For between-transformation noise (source 2) the standard deviation was calculated between the mean R-luc values coming from the two different inducible genes (DEX- and OHT-inducible) on the same day (as R-luc expression is controlled by the same repressible promoter in both gene circuits). Finally, the standard deviation of the mean luminescence between days for both F-luc and R-luc was calculated. This gives a measure of the batch effect (source 3).

### Example 10: Data Analysis

Data is processed in the following steps using MATLAB. (1) Camera corrected F-luc RLU/(area*s) and R-luc RLU/(area*s) values, and inducer type and concentration are stored in different .csv files for each promoter tested. (2) DEX and OHT data are separated. (3) Fold Change (FC) values are calculated for each promoter. Promoters with a FC>1.3 are stored for further analysis. (4) Data from promoters that do not meet the threshold criteria are tagged and kept for further processing. (5) RLU data are converted to Molecule Number/well via the RLU vs concentration standard curves shown in FIG. 2C-D. (6) The Mean F-luc values at zero inducer concentration of those plasmids showing a FC >1.3 are calculated for both DEX- and OHT-based systems. (7) Values of the normalization factor λᵢ are calculated using Eq. 3 (Main Text) and the data values of F-luc and R-luc molecule number divided by this factor. (8) Data are fit to the functional form given in Eq. 2 with different initial conditions for the nonlinear fit (12 different initial conditions were used to ensure convergence). If the fits converged to different minima, we chose the fit with the lowest p-value. The parameters of the fit are then stored, and can be used for further analysis.

### Example 10: Conclusion

The detailed analysis of promoter-repressor pairs in isolated plant cells attempts to determine if gene circuits with predictable behavior can be produced in multicellular eukaryotic organisms such as plants. The data in the examples were obtained from protoplast assays, where the levels of synthetic repressors were varied by using external inducers, and the quantitative behavior of the synthetic promoters was determined by measuring R-luc luminescence. The level of the repressor protein was measured by controlling F-luc by the same promoter. Readouts in the form of R-luc and F-luc luminescence provided quantitative measures of the promoter-repressor circuit.

The results show that quantitative data obtained from a rapid transient protoplast assay, when combined with rigorous analysis of noise and mathematical modeling, allows fast and quantitative estimation of the parameters of synthetic gene parts. The data show that it is possible to reliably assess repressor strength using the suite of experimental methods presented herein, and these quantitative measures stand up to experimental analysis in plants. The results support the mathematical model as a reasonable depiction of the experiment, suggesting that further direct measurements of the unknown terms in the equations could significantly reduce model uncertainties. The procedures described here therefore are immediately applicable for the development of comprehensive quantitatively characterized libraries of synthetic plant gene parts. The quantitative parameters of each promoter-repressor pair can be then used for in silico testing of the suitability of its use in more complex genetic circuits, such as a genetic toggle switch.

### Example 12: Supplementary Text

1. Luminescence Imaging Correction:

False-colored images of the 96 well plates with luminescing protoplasts appeared to show a systematic difference based on well position in the plate. To measure the extent of this difference we designed a simple “flip-plate” experiment. Two protoplast assays with 200 ul per well were collected for 5 minutes with well A1 in the top left hand corner and again for 5 minutes with well A1 in the bottom right hand corner. Only the first minute of each collection time was used to calculate the Relative Luminescence Units (RLU) for each well. We then calculated the percent change of F-Luc (in RLU/(area*s)) between the two values for each well. We repeated the experiment using purified recombinant F-Luc protein diluted to be in the range of our protoplast data. In both cases, we found significant differences between the measured luminescence of the two positions for the outer wells. The graph in FIG. 9B depicts the change in luminescence within the wells for one flip-plate experiment. The two measurements of the plate are superimposed, such that two measurements of the same well are plotted on top of each other (i.e., A1 when imaged near the top of camera's field of view, superimposed with A1 imaged near bottom of camera's field of view). As seen in the diagram, luminescence values of the wells on the left-hand side of the plate were consistently lower than values measured on the right-hand side of the plate. This experiment was repeated three times with results showing an average maximum percent change of 36% (+/−9%).

Since we image for five minutes (though only use the first minute of data for comparisons) we need to calculate the possible degradation of the luminescence signal during this time. Our data reveals that the degradation of the F-Luc signal has an average of 7% over a five-minute period (FIG. 8A); it can thus be assumed that F-Luc degradation would lead to a uniform signal degradation of about 7%.

We theorize that the most likely reason for these systematic imaging errors are that the camera does not pick up as many photons from wells that are farther away from its central axis when compared to wells that are closer. This could be seen from the dark crescents in the images themselves (FIG. 9C), suggesting a blocking effect by the non-transparent walls of the wells. The further away the well is from the projection of camera center on the plate, the larger the portion of its total volume is blocked by its wall, and consequently a smaller portion of the total F-Luc luminescence is collected by the camera. A post-imaging mathematical correction scheme was developed to correct the images based on a geometric calculation of this “missing volume” and careful physical measurements on our imaging system (details of the calculation are in FIG. 10). We developed a formula for the percentage difference between the original luciferase level and the level perceived by the camera for each of the wells on a plate, as a function of the distance between the geometric center of each well on the plate and the projection of camera center, given a chosen imaging shelf height.

Another complication arises when the camera center does not coincide with the center of the 96-well plate. We estimate where the camera center lies from the pattern of percent changes of each well on the plate, since the wells closest to the camera center should have the minimum percent changes (zero if the camera center lies directly above any well). We correct the luminescence data by using this formula to calculate the original luminescence of each well from its position on the plate and the observed intensity. We also built a frame for the 96-well plate that we used for all subsequent imaging in order to keep the plate center in a fixed position in relation to the camera center.

2. Image Correction Method:

The formula derived below for estimating the imaging correction is based on the 96-well plate geometry as shown in FIG. 10.

Step 1—Calculate the distance D between the center of the targeting well and projection of the camera center using similar triangles (FIG. 10A):

$$\frac{{h}_{1}}{{h}_{1} + {h}_{2}} = \frac{D - {r}_{1}}{D + l - {r}_{2}}$$

### Yields,

$$l = \frac{{h}_{2}}{{h}_{1}} ( D - {r}_{1} ) - ( {r}_{1} - {r}_{2} ) .$$

Then the upper edge of the well is projected to the bottom along the sight line between the camera and its closest point on the upper edge. 1 is the shift distance on the well bottom of the closest point along the sight line. Then the visible portion of the bottom edge is the part enclosed by the projection of the upper edge and itself. The area of this portion is calculated as follows.

This portion can be separated into two parts (A₁ and A₂) by the connecting line between the two intersections of the two circles. A₁ and A₂ can then be calculated using the differences of their corresponding sectors and triangles (FIG. 10B). Before the areas are calculated, the lengths of y₁, y₂ and x are needed via the equations listed as follows:

y₁+y₂=r₁−r₂+l

r₁²−y₁²=r₂²−y₂²=x²

Yields,

$${y}_{1} = \frac{{r}_{1}^{2} - {r}_{2}^{2} + {( {r}_{1} - {r}_{2} + l )}^{2}}{2 ( {r}_{1} - {r}_{2} + l )} = \frac{{r}_{1}^{2} - {r}_{2}^{2} + {a}^{2}}{2 a}$$

$${y}_{2} = a - {y}_{1} = \frac{{a}^{2} - {r}_{1}^{2} + {r}_{2}^{2}}{2 a}$$

$$x = \sqrt{{r}_{1}^{2} - {y}_{1}^{2}}$$

With a=r₁−r₂+l.

Based on these equations, the central angles of these two sectors can be calculated:

$${α}_{1} = arccos ( \frac{{y}_{1}}{{r}_{1}} )$$

$${α}_{2} = arccos ( \frac{{y}_{2}}{{r}_{2}} )$$

The areas of the two sectors can be expressed as: ½α₁r₁² and ½α₂r₂².

The two portions of the visible area on this plane can be calculated as:

A₁=½α₁r₁²−y₁x

A₂=½α₂r₂²−y₂x

The total visible area on the bottom is:

Aᵥ=A₁+A₂=½α₁r₁²−y₁x+½α₂r₂²−y₂x=½α₁r₁²+½α₂r₂²−αx.

To get the visible volume from the visible area, integration is needed from the bottom of the well to the liquid surface. Therefore, it is necessary to calculate the depth of the reagent inside the well. Taking note of the “imaginary cone” as shown in FIG. 10C, this integral can be set up using the 3 steps described below:
1) To calculate the height of the “imaginary cone” by similar triangles for the integration upper limit:

$$\frac{{h}_{i}}{{h}_{i} + {h}_{2}} = \frac{{r}_{2}}{{r}_{1}}$$

Calculate h as:

$${h}_{i} = \frac{{r}_{2} {h}_{2}}{{r}_{1} - {r}_{2}}$$

Also from another pair of similar triangles:

$$\frac{{h}_{i}}{{h}_{i} + d} = \frac{{r}_{2}}{r}$$

Results in:

$$r = \frac{{h}_{i} + d}{{h}_{i}} {r}_{2} = \frac{\frac{{r}_{2} {h}_{2}}{{r}_{1} - {r}_{2}} + d}{\frac{{r}_{2} {h}_{2}}{{r}_{1} - {r}_{2}}} {r}_{2} = \frac{{r}_{2} {h}_{2} + d ( {r}_{1} - {r}_{2} )}{{h}_{2}} = {r}_{2} + \frac{d}{{h}_{2}} ( {r}_{1} - {r}_{2} )$$

Reagent volumes are derived from the wet lab protocol and, this can be employed to calculate the depth using:

$$V ( d ) = \frac{1}{3} π {r}^{2} ( {h}_{i} + d ) - \frac{1}{3} π {r}_{2}^{2} {h}_{i} = \frac{1}{3} {π ( {r}_{2} + \frac{d}{{h}_{2}} ( {r}_{1} - {r}_{2} ) )}^{2} ( {h}_{i} + d ) - \frac{1}{3} π {r}_{2}^{2} {h}_{i}$$

2) Change the h₂ in the expression for bottom visible area into a variable s, as the distance between the top circle and the current plane. This gives the infinitesimal visible volume as:

Aᵥ(s)ds
3) Integrate these elements from the bottom of the well to the surface of the liquid to get the total visible volume as:

Vᵥₜₒₜₐₗ=∫ₕ_{₂}^{d}Aᵥ(s)ds=Vᵥₜₒₜₐₗ(D)

3. Conversion of Luminescence Values to Physical Units:

The function of the promoter-repressor pairs were experimentally characterized using luminescence from two types of luciferase. Luminescence values are typically reported in RLUs, or relative luciferase units. For the collection system (Stanford Photonics ICCD Camera), RLU is the sum of pixel intensity values within an area over collection time RLU/area*s), and represents the activity of F-Luc and R-Luc for each protoplast sample. RLUs were converted to molecules of luciferase by quantifying the relationship between the luminescence and the luciferase activity from purified recombinant F-Luc and R-Luc. FIG. 2C-D shows standard curves used to convert from RLU values to an absolute number of molecules for both F-Luc and R-Luc. The standard curves are linear, with high R² values (0.97, 0.96). We found that there is a difference in the number of molecules of R-Luc or F-Luc that generate the same RLU level. these standard curves were used with the image-corrected data to provide absolute molecule numbers for the mathematical analysis.

4. Testing the Sources of Noise:

Protoplast transformations were prepared with one DEX-inducible gene circuit and one OHT-inducible gene circuit (enough for 48 wells each). Luminescence data were collected with no inducer added, and repeated the experiment on three different days. In the absence of any noise, all wells should display identical F-Luc and identical R-Luc luminescence, with R-Luc expression at its maximum. Thus, variations between luminescence values from wells containing the same gene circuit on the same plate represent within-plate noise (the first source of noise). The difference between the mean R-Luc luminescence measured from the DEX-inducible gene circuit and the OHT-inducible gene circuit in the same batch represents the between-transformation noise (the second source of noise). Finally, the difference between the mean luminescence of the three batches represents the between-batch noise (the third source of noise)

Because the batch effect is a random variation that affects the entire population of protoplasts in a batch, it can be represented mathematically by a random number α such that the observed luminescence in the j-th well of the i-th batch can be represented by Rᵢⱼ=αᵢB_{R}+δᵢⱼ and Fᵢⱼ=αᵢB_{F}+δ′ᵢⱼ, for R-luc and F-luc luminescence, respectively. Here, B_{R}, B_{F} are the steady state number of luciferase molecules in the well in the absence of any noise for the R-luc and F-luc promoters, respectively; αᵢ is a random number that represents a multiplicative batch effect, while δᵢⱼ, δ′ᵢⱼ are random variables that represent additive noise terms that could arise from the remaining noise sources. If the R-luc and the F-luc luminescence are averaged for each batch and plot them, αᵢB_{R}+ [symbol] δᵢⱼ [symbol] ⱼ would be plotted against αᵢB_{F}+ [symbol] δ′ᵢⱼ [symbol] ⱼ (where the subscript on the angled brackets indicates the index being averaged). If this plot is approximately linear we can conclude that the batch effect is identical for both R-luc and F-luc, and dominates the additive noise terms.

5. Testing the Normalization Scheme with Simulated Data:

To generate simulated data, single-plasmid data were first generated using Equation 1 with assumed parameter values. Then the single-plasmid data was multiplied by a normally distributed random number representing the number of plasmids in each well (Nᵢⱼ) and another random number drawn from a log normal distribution representing the batch effect factor (αᵢ). The latter was assumed smaller than 1 based the our analysis in the main text.

For simplicity all the constants C₁, C₂ and \tilde{C} were set to 1. 1000 sets of data were simulated, consisting of six inducer levels and two technical replicates, similar to the experimental data. For each set one value of a was chosen from a log normal distribution with a mean less than one. Because the log normal distribution is unbounded in the positive infinity direction, a 95% cut-off for the distribution of a was assumed. To test the normalization scheme with different levels of noise, the standard deviation was increased to obtain a series of distributions with a decreasing population mean and increasing variance of αᵢ. Since each well in the experiments has approximately 10,000 protoplasts, this was set to be the mean of Nᵢⱼ and simulated various levels of noise by changing the standard deviation of the normal distribution.

The fitting procedure produced fits with an unreasonably high Hill coefficient at high levels of noise in the simulated data. We therefore imposed the criteria that the fitted Hill coefficient of the repressible promoter should lie between 1 and 6.

Due to the high levels of noise that ca be artificially generated in the simulated data, there are also “bad fits” within 1<n<6. These can be further characterized by unreasonably high fitted values of B which are far away from the well-formed distribution of most fitting results. It was observed that the fitting results of each parameter form log normal distributions similar to the assumed distribution of α. Therefore, logarithmic transformation were carried out to the fitted values of B and applied outlier tests following Peirce's method (Peirce, 1852). Specifically the R-code written by Dardis and Muller (r-forge.r-project.org/projects/pierce/) was used, which extends the development of Pierce's method by S. M. Ross in 2003 (Ross, 2003).

Fits that meet the criterion of n and pass the outlier tests are deemed successful and this defines the Number of Successes (NOS) among the 1000 repeats carried out. Within these biologically feasible results, the mean and standard deviation of the three fitted parameters are compared, namely B, H and n in Equation 2 in the main text.

The variation in the magnitude of the parameters is a measure of the effect of experimental noise on our estimates. The coefficient of variation of the estimated parameters was therefore plotted against the level of noise introduced in the simulated data in FIG. 4. It was determined that the alpha-normalization procedure can indeed reduce the coefficient of variation of the estimates of B and H between different log standard deviations of the alpha distribution, and thus make them more comparable. However the estimates of the Hill coefficient n are not improved by the alpha normalization.

6. Normalization for Comparison with Stably Transformed Plants:

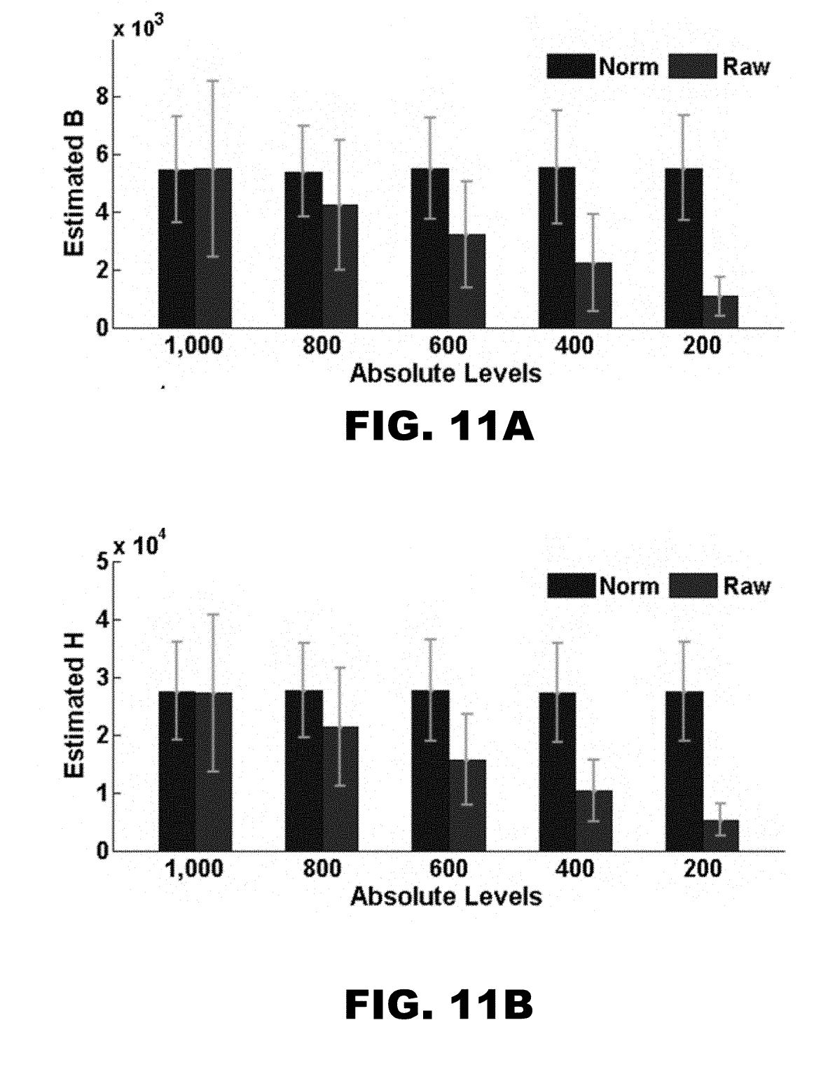

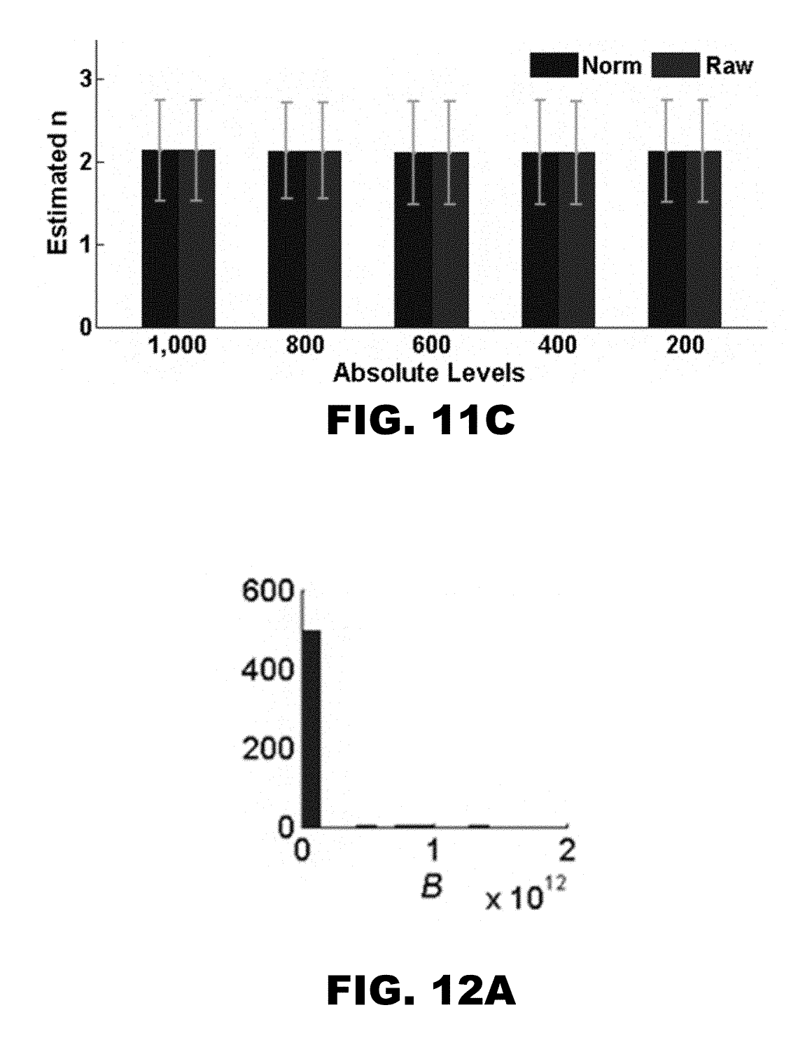

A key difference in the mathematical description of the stably transformed plants is the number of working circuits each protoplast contains. For the heterozygous plants used in our study, a single copy of the inducer-repressor circuit is expected, as suggested from genetic segregation data (not shown). However, for the transient protoplast assay it is expected that on average multiple copies of the plasmid would be found in each protoplast. The data shown in FIG. 6A-B support this expectation; in the no-inducer treatment R-luc luminescence levels are just over 4-fold smaller in protoplasts from stably transformed plants compared to transiently transformed protoplasts, despite the fact that the initial cell density of the former is five times greater than the latter. Since the parameter B in Equation 2 is proportional to the average number of viable plasmids [symbol] αᵢ [symbol] [symbol] Nᵢⱼ [symbol] for the construct, estimates of B from transient data are expected to be overestimates of the estimates of B for stable constructs. In agreement with this expectation, tests on simulated data with varying levels of mean Nᵢⱼ showed that Bᵢ was systematically overestimated as the mean number of plasmids became larger (FIG. 11). In order to correct for this overestimation we normalized our stably transformed plant data with the mean of the distribution coming from the transient assay. In other words, we defined a normalization factor λ*ᵢ such that

$${λ}_{i}^{*} = \frac{{〈 {F}_{ij}^{0 s} 〉}_{j}}{{〈 {F}_{ij}^{0 t} 〉}_{ij}} = \frac{{〈 {N}_{ij}^{0 s} 〉}_{j} {α}_{i}}{{〈 {N}_{ij}^{0 t} 〉}_{ij} 〈 {α}_{i} 〉} \qquad 4 )$$

Here, the superscript 0t refers to the zero-inducer values for the transient assay, and 0s refers to the zero inducer values of the stable transformation assay. Dividing the data by λ*ᵢ therefore not only replaces αᵢ by [symbol] αᵢ [symbol] but multiplies each F-luc and R-luc value by the fraction by which the plasmids in an average well in the transient assay exceed those in the stably transformed assay (i.e. the fraction [symbol] Nᵢⱼ^{0t} [symbol] ᵢⱼ [symbol] Nᵢⱼ^{0s} [symbol] ⱼ). Tests on simulated data (FIG. 11) show that the estimates of B and H obtained by this method are insensitive to changes in the mean of the plasmid number Nᵢⱼ and therefore allow comparison of transient assays with stably transformed assays.

7. Testing the Normalization Factor λ*ᵢ with Simulated Data:

As described elsewhere, there is only a single copy of the repressible promoter circuit in the stably transformed transgenic plant cells and multiple copies of the plasmid are expected in each transiently transformed protoplast. This leads to different multipliers found in parameter B in Equation 2 and hinders the direct comparisons of the estimated parameter values between transient and stably transformed assays. In the main text, we proposed a normalization factor λ*ᵢ to correct this bias from plasmid numbers. We will test if this normalization factor λ*ᵢ behaves as expected using simulated data similarly to what we did for FIG. 4.

Due to the natural differences between the transient and stably transformed assays, we assume the noise levels are positively proportional to the mean numbers of plasmid in each protoplast. Nᵢⱼ of the transient assays is assumed to be high in both mean and variance, and Nᵢⱼ of the stably transformed assays to be low in both. Therefore, we assume the coefficient of variance (COV) is the same between the transient and stably transformed assays, while the absolute levels are different. Therefore, instead of varying the standard deviation of the normal distribution underlying Nᵢⱼ while keeping the mean value the same in FIG. 4, we varied the standard deviation and mean values at the same time and kept the COV the same. To observe the trend clearly, we carried out simulations with five decreasing absolute noise levels. We then normalized the simulated data at each level with the [symbol] Fᵢⱼ⁰ [symbol] ⱼ from the case with highest mean value (simulated transient assays) rather than with their corresponding [symbol] Fᵢⱼ⁰ [symbol] ⱼ as in previous simulations (Equation 3 in the main test). We applied the same fitting procedure and measurements to both normalized dataset and its corresponding raw dataset. The results are shown in FIG. 11. Blue bars are fitting results of normalized data while red bars represent the raw data. Similar to what was observed in FIG. 4, fitting results of n is insensitive to the normalization we applied. Decreases in mean fitted values for B and H are observed for the raw data. In contrast the mean fitted values for B and H in the normalized data are at similar levels across all five noise levels simulated. This shows the proposed normalization factor λ*ᵢ meets the expectation and makes different absolute levels comparable.

8. Bootstrapping Data Analysis of Transient Vs Stable Transformants:

Bootstrapping statistical analysis was carried out to generate mean values and confidence intervals for the predictions in stably transformed plants. Bootstrapping is a useful inference method when the underlying distribution of the data is not known or when the sample size is small (Fox, 2008).

As shown elsewhere: (1) The predictions for B are quite accurate in one of the three cases, and overestimated only by a factor of three or four in the other two. (2) The predictions for H are very good in one of the three cases and within a factor of five for the third. (3) The predictions for n also lie within a factor of two or three of the value for the plant data. Bootstrapping then looks into the question: if the data had been sampled differently would the predictions still look the same?

To generate the different sample sets, the original data set was randomly selected to form bootstrap sample sets in the following 3 steps. First the appropriate number of bins to histogram F-Luc values was chosen. The chosen number of bins was the largest number that yielded no bins with zero values in it. This was done to optimize the sampling of the data. Next the number of sample points to draw from each bin was chosen. This was set to be one greater than the minimum number of points in any bin. For example, if one bin in the F-Luc histogram contained only one point, the maximum number of sample points that could be drawn from any bin was set as 2. This was done to avoid drawing the same point an excessive number of times per sample. Histograms were then made of the F-luc data with the number of bins chosen in step 1, and the corresponding R-luc data was placed in a corresponding R-luc bin. Bootstrapped samples were created by drawing the number of sample points fixed in step 2 from each bin, randomly and with replacement.

500 bootstrap samples were created separately from transient and stable data for each construct. Each bootstrap sample was fit using the standard procedure, and the parameters B, H and n estimated. This exercise produces a distribution of fitted values for each parameter. In FIG. 12A-C, these distributions appear to have large outliers. Outliers that were 3 standard deviations or greater away from the mean were identified and removed. Mean values and confidence intervals were then calculated from the remaining distribution. The lower and upper bound for each confidence interval is then the 5% and 95% values from the final bootstrapped distribution, giving a 90% confidence interval.

The results of the bootstrapping exercise are shown in FIG. 12D-F. To summarize these results: (1) The predictions for B appear to be in the same range and show the same trend as the original fits; (2) The predictions for H appear to be in the same range and are comparable if not more comparable between plant and transient data as in the original fits; and (3) The mean value for the predictions for n lies within at least a factor of 2.56 between the stable and transient data. However the increased confidence intervals suggest that this parameter is harder to recover, as suggested by the simulated data.

### REFERENCES

1 Kiani, S. et al. CRISPR transcriptional repression devices and layered circuits in mammalian cells. Nat Methods, doi:10.1038/nmeth.2969 (2014).
2 Rinaudo, K. et al. A universal RNAi-based logic evaluator that operates in mammalian cells. Nat Biotechnol 25, 795-801 (2007).
3 Gardner, T. S., Cantor, C. R. & Collins, J. J. Construction of a genetic toggle switch in Escherichia coli. Nature 403, 339-342 (2000).
4 You, L., Cox, R. S., 3rd, Weiss, R. & Arnold, F. H. Programmed population control by cell-cell communication and regulated killing. Nature 428, 868-871 (2004).
5 Steeves, T. A. & Sussex, I. M. Patterns in plant development. 2 edn, (Cambridge University Press, 1989).
6 Feng, S., Jacobsen, S. E. & Reik, W. Epigenetic reprogramming in plant and animal development. Science 330, 622-627, doi:10.1126/science.1190614 (2010).
7 Kim, J., Klein, P. G. & Mullet, J. E. Synthesis and turnover of photosystem II reaction center protein Dl. Ribosome pausing increases during chloroplast development. J Biol Chem 269, 17918-17923 (1994).
8 Asai, T. et al. MAP kinase signalling cascade in Arabidopsis innate immunity. Nature 415, 977-983 (2002).
9 Mewes, H. W. et al. Overview of the yeast genome. Nature 387, 7-65. (1997).
10 Klein, T., M., Wolf, E. D., Wu, R. & Sanford, J. C. High-velocity microprojectiles for delivering nucleic acids into living cells. Nature 327, 70-73 (1987).
11 Wu, H. Y. et al. AGROBEST: an efficient Agrobacterium-mediated transient expression method for versatile gene function analyses in Arabidopsis seedlings. Plant methods (2014).
12 Bargmann, B. O. & Birnbaum, K. D. Fluorescence activated cell sorting of plant protoplasts. J Vis Exp, doi:10.3791/1673 (2010).
13 Lucks, J. B., Qi, L., Whitaker, W. R. & Arkin, A. P. Toward scalable parts families for predictable design of biological circuits. Current opinion in microbiology 11, 567-573, doi:10.1016/j.mib.2008.10.002 (2008).
14 Slusarczyk, A. L., Lin, A. & Weiss, R. Foundations for the design and implementation of synthetic genetic circuits. Nature reviews. Genetics 13, 406-420, doi:10.1038/nrg3227 (2012).
15 Wang, S., Chang, Y., Guo, J. & Chen, J.-G. Arabidopsis Ovate Family Protein 1 is a transcriptional repressor that suppresses cell elongation. The Plant journal: for cell and molecular biology 50, 858-872, doi:10.1111/j.1365-313X.2007.03096.x (2007).
16 Wang, S. et al. Arabidopsis ovate family proteins, a novel transcriptional repressor family, control multiple aspects of plant growth and development. PloS one 6, e23896, doi:10.1371/journal.pone.0023896 (2011).
17 Ikeda, M. & Ohme-Takagi, M. A novel group of transcriptional repressors in Arabidopsis. Plant and Cell Physiology (2009).
18 Ohta, M., Matsui, K., Hiratsu, K. & Shinshi H. & Ohme-Tagagi, M. Repression domains of class II ERF transcriptional repressors share an essential motif for active repression. The Plant Cell (2001).
19 Odell, J., Nagy, F. & Chua, N. Identification of DNA sequences required for activity of the cauliflower mosaic virus 35S promoter. Nature (1985).
20 Sanger, M., Daubert, S. & Goodman, R. Characteristics of a strong promoter from figwort mosaic virus: comparison with the analogous 35S promoter from cauliflower mosaic virus and the regulated mannopine synthase promoter. Plant Molecular Biology (1990).
21 Shaw, C., Carter, G. & Watson, M. A functional map of the nopaline synthase promoter. Nucleic acids research (1984).
22 Aoyama, T. & Chua, N. A glucocorticoid-mediated transcriptional induction system in transgenic plants. The Plant Journal (1997).
23 Zuo, J., Niu, Q. & Chua, N. An estrogen receptor-based transactivator XVE mediates highly inducible gene expression in transgenic plants. The Plant Journal (2000).
24 Alon, U. An introduction to systems biology: design principles of biological circuits. (Chapman & Hall/CRC, 2007).
25 Padidam, M. & Cao, Y. Elimination of transcriptional interference between tandem genes in plant cells. Biotechniques (2001).
26 Depicker, A., Stachel, S., Dhaese P., Zambryski P. & Goodman, H. M. Nopaline synthase: transcript mapping and DNA sequence. Journal of molecular and applied genetics. (1982).
27 Engler, C., Gruetzner, R., Kandzia, R. & Marillonnet, S. Golden gate shuffling: a one-pot DNA shuffling method based on type IIs restriction enzymes. PloS one (2009).
28 Antunes, M. S. et al. A synthetic de-greening gene circuit provides a reporting system that is remotely detectable and has a re-set capacity. Plant biotechnology journal 4, 605-622, doi:10.1111/j.1467-7652.2006.00205.x (2006).
29 Samalova, M., Brzobohaty, B. & Moore, I. pOp6/LhGR: a stringently regulated and highly responsive dexamethasone-inducible gene expression system for tobacco. Plant J 41, 919-935, doi:10.1111/j.1365-313X.2005.02341.x (2005).
30 Sakuma, Y. et al. Functional analysis of an Arabidopsis transcription factor, DREB2A, involved in drought-responsive gene expression. Plant Cell 18, 1292-1309, doi:10.1105/tpc.105.035881 (2006).
31 Yoo, S., Cho, Y. & Sheen, J. Arabidopsis mesophyll protoplasts: a versatile cell system for transient gene expression analysis. Nature protocols (2007).
32 Peirce, B. Criterion for the rejection of doubtful observations. The Astromomical Journal 2(45), 161-163 (1852).
33 Ross, S. Peirce's criterion for the elimination of suspect experimental data. Journal of Engineering Technology. 1-12 (2003).
34 Fox, J. Applied Regression Analysis and Generalized Linear Models. (Sage Publications, Thousand Oaks, Calif., 2008).

What is claimed is:

1. A synthetic repressor construct for modifying gene expression in a plant, comprising a nucleic acid encoding a transcriptional repressor domain linked to a DNA-binding domain, wherein the transcriptional repressor domain and the DNA-binding domain are operable in the same plant species.

2. The synthetic repressor construct of claim 1, wherein the DNA-binding domain is a sequence specific DNA-binding domain.

3. The synthetic repressor construct of claim 2, wherein the DNA-binding domain is selected from a yeast Gal4 DNA-binding domain and a bacterial LexA DNA-binding domain.

4. The synthetic repressor construct of claim 1, wherein the transcriptional repressor domain is selected from an EAR transcriptional repressor domain, an OFP transcriptional repressor domain, or a BRD transcriptional repressor domain.

5. The synthetic repressor construct of claim 4, wherein the OFPx transcriptional repressor domain comprises SEQ ID NO: 35.

6. The synthetic repressor construct of claim 1, wherein the transcriptional repressor domain is an OFP transcriptional repressor domain and the DNA-binding domain is a bacterial LexA DNA-binding domain.

7. The synthetic repressor construct of claim 1, wherein the transcriptional repressor domain is a BRD transcriptional repressor domain and the DNA-binding domain is a yeast Gal4 DNA-binding domain.

8. The synthetic repressor construct of claim 1, wherein the nucleic acid encoding a transcriptional repressor domain linked to a DNA-binding domain nucleic acid is selected from the group consisting of SEQ ID NO: 27-34.

9. A synthetic repressible promoter construct for use in combination with a synthetic repressor construct of claim 1, the synthetic repressible promoter construct comprising:
(a) a nucleic acid sequence encoding a core promoter capable of conferring constitutive gene expression in a plant species, the core promoter optionally comprising a TATA box; and
(b) a synthetic regulatory element comprising at least one copy of a binding element having a nucleic acid sequence capable of specifically binding the DNA-binding domain of the synthetic repressor, the copy of the at least one binding element inserted at a position upstream of the core promoter, downstream of the core promoter but before the translation start site for a protein of interest, or proximal to the 5′ end of the optionally present TATA box when present.

10. The synthetic repressible promoter construct of claim 9, wherein the core promoter is selected from the group consisting of Cauliflower Mosaic Virus (CaMV35S) promoter, Figwort Mosaic Virus (FMV) promoter, Nopaline Synthase (NOS) promoter, Ubiquitin-1 promoter from maize (ZmUBI1), and Actin 2.1 promoter from rice (OsACT2.1).

11. The synthetic repressible promoter construct of claim 9, wherein the synthetic regulatory element comprises at least 2 and no more than 10 copies of at least one binding element.

12. The synthetic repressible promoter construct of claim 11, wherein two or more copies of the binding element are separated from each other by a nucleic acid spacer sequence having a length of about 2 to about 10 nucleotides.

13. The synthetic repressible promoter construct of claim 9, wherein the binding element specifically binds a yeast Gal4 DNA-binding domain or a bacterial LexA DNA-binding domain.

14. The synthetic repressible promoter construct of claim 9 comprising a nucleic acid selected from the group consisting of SEQ ID NO: 1-26.

15. A synthetic repressible promoter construct of claim 9 operably linked to a nucleic acid encoding a protein of interest.

16. A synthetic repressible promoter construct of claim 9, wherein the 3′ end of the core promoter is proximal to a cloning site

17. An artificial genetic circuit for modifying expression of a protein of interest in a plant, comprising:
(a) a promoter operably linked to a synthetic repressor construct, the synthetic repressor construct comprising a nucleic acid encoding a transcriptional repressor domain linked to a DNA-binding domain;
(b) a nucleic acid construct comprising a nucleic acid encoding a protein of interest;
(c) a synthetic repressible promoter construct operably linked to the nucleic acid encoding the protein of interest, the synthetic repressible promoter construct comprising: (i) a nucleic acid sequence encoding a core promoter capable of conferring constitutive gene expression in a plant species, the core promoter optionally comprising a TATA box, and (ii) a synthetic regulatory element comprising at least one copy of a binding element having a nucleic acid sequence capable of specifically binding the DNA-binding domain of the synthetic repressor, the copy of the at least one binding element inserted at a position upstream of the core promoter, downstream of the core promoter region but before a translation start site for the protein of interest, or proximal to the 5′ end of the optionally present TATA box when present; and
wherein the transcriptional repressor domain of the synthetic repressor, the DNA-binding domain of the synthetic repressor, the promoter operably linked to the synthetic repressor construct, and the core promoter of the synthetic repressible construct are each operable in the same plant species.

18. The artificial genetic circuit of claim 17, wherein the transcriptional repressor domain is selected from an EAR transcriptional repressor domain, an OFP transcriptional repressor domain, or a BRD transcriptional repressor domain.

19. The artificial genetic circuit of claim 18, wherein the OFPx transcriptional repressor domain comprises SEQ ID NO: 35.

20. The artificial genetic circuit of claim 17, wherein the DNA-binding domain of the synthetic repressor is a yeast Gal4 DNA-binding domain, and the binding element of the synthetic repressible promoter construct specifically binds the yeast Gal4 DNA-binding domain.

21. The artificial genetic circuit of claim 17, wherein the DNA-binding domain of the synthetic repressor is a bacterial LexA DNA-binding domain and the binding element of the synthetic repressible promoter construct specifically binds the bacterial LexA DNA-binding domain.

22. The artificial genetic circuit of claim 17, wherein the core promoter is selected from the group consisting of Cauliflower Mosaic Virus (CaMV35S), Figwort Mosaic Virus (FMV), and Nopaline Synthase (NOS)] promoters.

23. The artificial genetic circuit of claim 17, wherein the transcriptional repressor domain is an OFP transcriptional repressor domain and the DNA-binding domain is a bacterial LexA DNA-binding domain.

24. The genetic circuit of claim 17, wherein the core promoter is cauliflower mosaic virus 35S promoter.

25. The artificial genetic circuit of claim 17, wherein the transcriptional repressor domain is a BRD transcriptional repressor domain and the DNA-binding domain is a yeast Gal4 DNA-binding domain.

26. The artificial genetic circuit of claim 17, wherein the synthetic regulatory element comprises two or more copies of the binding element having a nucleic acid sequence capable of specifically binding the nucleic acid binding domain of the synthetic repressor.

27. The artificial genetic circuit of claim 17, wherein the synthetic regulatory element comprise at least 2 and no more than 10 copies of the at least one binding element.

28. The artificial circuit of claim 27, wherein two or more copies of the binding element are separated from each other by a nucleic acid spacer sequence having a length of about 2 to about 10 nucleotides.

29. A transgenic plant cell comprising the artificial genetic circuit of claim 17.

30. The transgenic plant cell of claim 29, wherein the plant cell is a leaf cell.

31. The transgenic plant cell of claim 29, wherein the plant cell is a root cell.

32. The transgenic plant cell of claim 29, wherein the plant cell is a crop plant cell.

33. A kit comprising a plurality of synthetic gene circuits of claim 17, wherein each synthetic gene circuit of the kit varies from the other synthetic gene circuits in the number of binding elements at a given position and/or the spacing between the binding elements at a given position.

34. The kit of claim 33, wherein each synthetic gene circuit has the same transcriptional repressor domain, DNA-binding domain, and constitutive promoter.

35. A method for modifying expression of a protein of interest in a plant, the method comprising introducing the synthetic genetic circuit of claim 17 into a cell of the plant, wherein the promoter operably linked to the synthetic repressor construct is an inducible promoter.

36. The method of claim 35, wherein the cell is a leaf cell.

37. The method of claim 35, wherein the cell is a root cell.

38. The method of claim 35, wherein the cell is a crop cell.

39. The method of claim 35, further comprising varying the level of expression of the protein of interest across different cell types or tissues types in the plant by varying the number of binding elements at a given position and/or the spacing between the 2 or more binding elements at a given position.

40. A method for creating a library comprising a plurality of synthetic repressible promoter constructs, the method comprising: providing a construct comprising a core promoter capable of conferring constitutive gene expression in a plant species, wherein the promoter optionally comprises a TATA box, and modifying the construct a plurality of times by (a) introducing one or more copies of a binding element having a nucleic acid sequence capable of specifically binding a DNA-binding domain, the copy of the binding element inserted at a position upstream of the core promoter, downstream of the core promoter but before a translation start site for a protein of interest, or proximal to the 5′ end of the optionally present TATA box when present, and then (b) varying the number of binding elements at a given position and/or the spacing between the 2 or more binding elements at a given position.

41. The method of claim 40, wherein the construct is provided in the form of a vector, and the vector further comprises a synthetic repressor construct, the synthetic repressor construct comprising a promoter operably linked to a nucleic acid encoding a transcriptional repressor domain and a DNA-binding domain; wherein the DNA-binding domain specifically binds to a binding element of the synthetic repressible promoter construct, and wherein the transcriptional repressor domain of the synthetic repressor, the DNA-binding domain of the synthetic repressor, the promoter operably linked to the synthetic repressor construct, and the core promoter of the synthetic repressible construct are each operable in the same plant species.

42. A transgenic plant comprising a transgenic plant cell of claim 29.


SEQUENCE LISTING (35 sequences)

SEQ ID NO: 1 - DNA, 623 bp, Artificial Sequence
  SYNTHESIZED
cggaagactctcctccgagcggaagactctcctccgaagcttaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacg

SEQ ID NO: 2 - DNA, 364 bp, Artificial Sequence
  SYNTHESIZED
cggaagactctcctccgagcggaagactctcctccgaagcttaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccg

SEQ ID NO: 3 - DNA, 386 bp, Artificial Sequence
  SYNTHESIZED
cggaagactctcctccgagcggaagactctcctccgaagctttgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 4 - DNA, 394 bp, Artificial Sequence
  SYNTHESIZED
cggaagactctcctccgtaatgcttcgcggaagactctcctccgaagctttgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 5 - DNA, 642 bp, Artificial Sequence
  SYNTHESIZED
cctagtagcttccggaagactctcctccgtaatgcttcgcggaagactctcctccgaagcttaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaac

SEQ ID NO: 6 - DNA, 372 bp, Artificial Sequence
  SYNTHESIZED
cggaagactctcctccgtaatgcttcgcggaagactctcctccgaagcttaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccg

SEQ ID NO: 7 - DNA, 386 bp, Artificial Sequence
  SYNTHESIZED
actgtacatataaccactggttttatatacagcagtaagctttgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 8 - DNA, 623 bp, Artificial Sequence
  SYNTHESIZED
actgtacatataaccactggttttatatacagcagtaagcttaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacg

SEQ ID NO: 9 - DNA, 364 bp, Artificial Sequence
  SYNTHESIZED
actgtacatataaccactggttttatatacagcagtaagcttaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccg

SEQ ID NO: 10 - DNA, 386 bp, Artificial Sequence
  SYNTHESIZED
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaacgcgtcggaagactctcctccgagcggaagactctcctccg

SEQ ID NO: 11 - DNA, 406 bp, Artificial Sequence
  SYNTHESIZED
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaacgcgtcctagtagcttccggaagactctcctccgtaatgcttcgcggaagactctcctccg

SEQ ID NO: 12 - DNA, 386 bp, Artificial Sequence
  SYNTHESIZED
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaacgcgtactgtacatataaccactggttttatatacagcagt

SEQ ID NO: 13 - DNA, 416 bp, Artificial Sequence
  SYNTHESIZED
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctcactgtacatataaccactggttttatatacagcagtactgtacatataaccactggttttatatacagcagttatataaggaagttcatttcatttggagagga

SEQ ID NO: 14 - DNA, 437 bp, Artificial Sequence
  SYNTHESIZED
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctccggaagactctcctccgagcggaagactctcctccgagcggaagactctcctccaggcggaagactctcctccgagcggaagactctcctccgtatataaggaagttcatttcatttggagagga

SEQ ID NO: 15 - DNA, 738 bp, Artificial Sequence
  SYNTHESIZED
actgtacatataaccactggttttatatacagcagtactgtacatataaccactggttttatatacagcagtcgacgtactgtacatataaccactggttttatatacagcagtactgtacatataaccactggttttatatacagcagtcaagcttaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacg

SEQ ID NO: 16 - DNA, 501 bp, Artificial Sequence
  SYNTHESIZED
actgtacatataaccactggttttatatacagcagtactgtacatataaccactggttttatatacagcagtcgacgtactgtacatataaccactggttttatatacagcagtactgtacatataaccactggttttatatacagcagtcaagctttgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 17 - DNA, 479 bp, Artificial Sequence
  SYNTHESIZED
actgtacatataaccactggttttatatacagcagtactgtacatataaccactggttttatatacagcagtcgacgtactgtacatataaccactggttttatatacagcagtactgtacatataaccactggttttatatacagcagtcaagcttaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccg

SEQ ID NO: 18 - DNA, 528 bp, Artificial Sequence
  SYNTHESIZED
actgtacatataaccactggttttatatacagcagttaatgcttcgactgtacatataaccactggttttatatacagcagttaatgcttcgactgtacatataaccactggttttatatacagcagttaatgcttcgactgtacatataaccactggttttatatacagcagtatcgagagacctgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 19 - DNA, 760 bp, Artificial Sequence
  SYNTHESIZED
actgtacatataaccactggttttatatacagcagttaatgcttcgactgtacatataaccactggttttatatacagcagttaatgcttcgactgtacatataaccactggttttatatacagcagttaatgcttcgactgtacatataaccactggttttatatacagcagtaagcttaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaac

SEQ ID NO: 20 - DNA, 507 bp, Artificial Sequence
  SYNTHESIZED
actgtacatataaccactggttttatatacagcagttaatgcttcgactgtacatataaccactggttttatatacagcagttaatgcttcgactgtacatataaccactggttttatatacagcagttaatgcttcgactgtacatataaccactggttttatatacagcagtatcgagagaccaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccg

SEQ ID NO: 21 - DNA, 623 bp, Artificial Sequence
  SYNTHESIZED
aggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacgacgcgtcggaagactctcctccgagcggaagactctcctccg

SEQ ID NO: 22 - DNA, 643 bp, Artificial Sequence
  SYNTHESIZED
aggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacgacgcgtcctagtagcttccggaagactctcctccgtaatgcttcgcggaagactctcctccg

SEQ ID NO: 23 - DNA, 623 bp, Artificial Sequence
  SYNTHESIZED
aggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaacgacgcgtactgtacatataaccactggttttatatacagcagt

SEQ ID NO: 24 - DNA, 364 bp, Artificial Sequence
  SYNTHESIZED
aggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccgacgcgtcggaagactctcctccgagcggaagactctcctccg

SEQ ID NO: 25 - DNA, 384 bp, Artificial Sequence
  SYNTHESIZED
aggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccgacgcgtcctagtagcttccggaagactctcctccgtaatgcttcgcggaagactctcctccg

SEQ ID NO: 26 - DNA, 364 bp, Artificial Sequence
  SYNTHESIZED
aggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccgacgcgtactgtacatataaccactggttttatatacagcagt

SEQ ID NO: 27 - DNA, 468 bp, Artificial Sequence
  SYNTHESIZED
atgaagttgttaagttctattgaacaggcttgtgatatttgtagattgaagaagctcaagtgtagtaaagagaaacctaagtgcgctaagtgtcttaaaaataactgggagtgcagatattctcctaagactaagagatcacctcttactagggctcatctcacagaggtggagtctaggcttgagagattggagcagttgttccttttgatttttccaagagaagatcttgatatgattcttaagatggattctcttcaagatattaaggctcttcttactggtttattcgttcaagataatgtgaataaggatgcagtgactgatagacttgcatcagttgaaactgatatgcctcttacattaagacaacacaggatttctgctacttcaagttctgaagaaagttctaataagggtcaaaggcaattaaccgtttctttaagactgttcggagtgaacatgtga

SEQ ID NO: 28 - DNA, 480 bp, Artificial Sequence
  SYNTHESIZED
atgaagttgttaagttctattgaacaggcttgtgatatttgtagattgaagaagctcaagtgtagtaaagagaaacctaagtgcgctaagtgtcttaaaaataactgggagtgcagatattctcctaagactaagagatcacctcttactagggctcatctcacagaggtggagtctaggcttgagagattggagcagttgttccttttgatttttccaagagaagatcttgatatgattcttaagatggattctcttcaagatattaaggctcttcttactggtttattcgttcaagataatgtgaataaggatgcagtgactgatagacttgcatcagttgaaactgatatgcctcttacattaagacaacacaggatttctgctacttcaagttctgaagaaagttctaataagggtcaaaggcaattaaccgtttctcttgatttggacctcgagcttcgtttgggattcgcttga

SEQ ID NO: 29 - DNA, 642 bp, Artificial Sequence
  SYNTHESIZED
atgaagttgttaagttctattgaacaggcttgtgatatttgtagattgaagaagctcaagtgtagtaaagagaaacctaagtgcgctaagtgtcttaaaaataactgggagtgcagatattctcctaagactaagagatcacctcttactagggctcatctcacagaggtggagtctaggcttgagagattggagcagttgttccttttgatttttccaagagaagatcttgatatgattcttaagatggattctcttcaagatattaaggctcttcttactggtttattcgttcaagataatgtgaataaggatgcagtgactgatagacttgcatcagttgaaactgatatgcctcttacattaagacaacacaggatttctgctacttcaagttctgaagaaagttctaataagggtcaaaggcaattaaccgtttctagcagggcggtggtgaaggcgtcggtggatccgaagagagactttaaggagtcgatggaggagatgatcgctgagaacaagataagagcgacaaaggatctagaggagcttttggcttgctatctctgtctcaattcagacgaatatcacgctattatcatcaatgtcttcaagcaaatctggcttgatcttaatcttcca

SEQ ID NO: 30 - DNA, 642 bp, Artificial Sequence
  SYNTHESIZED
atgaagttgttaagttctattgaacaggcttgtgatatttgtagattgaagaagctcaagtgtagtaaagagaaacctaagtgcgctaagtgtcttaaaaataactgggagtgcagatattctcctaagactaagagatcacctcttactagggctcatctcacagaggtggagtctaggcttgagagattggagcagttgttccttttgatttttccaagagaagatcttgatatgattcttaagatggattctcttcaagatattaaggctcttcttactggtttattcgttcaagataatgtgaataaggatgcagtgactgatagacttgcatcagttgaaactgatatgcctcttacattaagacaacacaggatttctgctacttcaagttctgaagaaagttctaataagggtcaaaggcaattaaccgtttctagcttggcggtggtgaagaagtcggtggatccaaacaaagatttcagggaatcaatggtggagatgatagcagagaacaagataagagcatcaaatgacctagaagagcttcttgcttgctacctttcgttaaatccaaaggaatatcacgatcttattatcaaagtgttcgaacaaatctggcttgaactgataaaccca

SEQ ID NO: 31 - DNA, 288 bp, Artificial Sequence
  SYNTHESIZED
atgaaagctcttactgctagacaacaagaagtttttgatttgattagggatcatatttctcagacaggtatgcctcctactagagctgagatcgctcagagactcggtttcagatctcctaacgctgctgaagagcatcttaaggctcttgctagaaagggagttattgagattgtgagtggagcatcaagaggtattaggttgcttcaagaggaagaagagggacttcctcttgttggtagagttgcagctggtgagcctttaagactgttcggagtgaacatgtga

SEQ ID NO: 32 - DNA, 300 bp, Artificial Sequence
  SYNTHESIZED
atgaaagctcttactgctagacaacaagaagtttttgatttgattagggatcatatttctcagacaggtatgcctcctactagagctgagatcgctcagagactcggtttcagatctcctaacgctgctgaagagcatcttaaggctcttgctagaaagggagttattgagattgtgagtggagcatcaagaggtattaggttgcttcaagaggaagaagagggacttcctcttgttggtagagttgcagctggtgagcctcttgatttggacctcgagcttcgtttgggattcgcttga

SEQ ID NO: 33 - DNA, 462 bp, Artificial Sequence
  SYNTHESIZED
atgaaagctcttactgctagacaacaagaagtttttgatttgattagggatcatatttctcagacaggtatgcctcctactagagctgagatcgctcagagactcggtttcagatctcctaacgctgctgaagagcatcttaaggctcttgctagaaagggagttattgagattgtgagtggagcatcaagaggtattaggttgcttcaagaggaagaagagggacttcctcttgttggtagagttgcagctggtgagcctagcagggcggtggtgaaggcgtcggtggatccgaagagagactttaaggagtcgatggaggagatgatcgctgagaacaagataagagcgacaaaggatctagaggagcttttggcttgctatctctgtctcaattcagacgaatatcacgctattatcatcaatgtcttcaagcaaatctggcttgatcttaatcttcca

SEQ ID NO: 34 - DNA, 462 bp, Artificial Sequence
  SYNTHESIZED
atgaaagctcttactgctagacaacaagaagtttttgatttgattagggatcatatttctcagacaggtatgcctcctactagagctgagatcgctcagagactcggtttcagatctcctaacgctgctgaagagcatcttaaggctcttgctagaaagggagttattgagattgtgagtggagcatcaagaggtattaggttgcttcaagaggaagaagagggacttcctcttgttggtagagttgcagctggtgagcctagcttggcggtggtgaagaagtcggtggatccaaacaaagatttcagggaatcaatggtggagatgatagcagagaacaagataagagcatcaaatgacctagaagagcttcttgcttgctacctttcgttaaatccaaaggaatatcacgatcttattatcaaagtgttcgaacaaatctggcttgaactgataaaccca

SEQ ID NO: 35 - DNA, 201 bp, Artificial Sequence
  SYNTHESIZED
agcttggcggtggtgaagaagtcggtggatccaaacaaagatttcagggaatcaatggtggagatgatagcagagaacaagataagagcatcaaatgacctagaagagcttcttgcttgctacctttcgttaaatccaaaggaatatcacgatcttattatcaaagtgttcgaacaaatctggcttgaactgataaaccca